SEQUENCE LISTING (22 sequences)

SEQ ID NO: 1 - protein, 252 aa, Streptococcus pneumoniae
MFGFFKKDKAVEVEVPTQVPAHIGIIMDGNGRWAKKRMQPRVFGHKAGMEALQTVTKAANKLGVKVITVYAFSTENWTRPDQEVKFIMNLPVEFYDNYVPELHANNVKIQMIGETDRLPKQTFEALTKAEELTKNNTGLILNFALNYGGRAEITQALKLISQDVLDAKINPGDITEELIGNYLFTQHLPKDLRDPDLIIRTSGELRLSNFLPWQGAYSELYFTDTLWPDFDEAALQEAILAYNRRHRRFGGV

SEQ ID NO: 2 - protein, 6 aa, Streptococcus pneumoniae
  misc_feature (4)..(4): Xaa can be any naturally occurring amino acid
ENWXRP

SEQ ID NO: 3 - protein, 5 aa, Streptococcus pneumoniae
  misc_feature (4)..(4): Xaa can be any naturally occurring amino acid
DGNXR

SEQ ID NO: 4 - protein, 6 aa, Streptococcus pneumoniae
DGNGRW

SEQ ID NO: 5 - protein, 6 aa, Streptococcus pneumoniae
ENWTRP

SEQ ID NO: 6 - protein, 6 aa, Streptococcus pneumoniae
PRVFGH

SEQ ID NO: 7 - protein, 6 aa, Streptococcus pneumoniae
RLSNFL

SEQ ID NO: 8 - protein, 6 aa, Streptococcus pneumoniae
HIGIIM

SEQ ID NO: 9 - protein, 5 aa, Streptococcus pneumoniae
GRWAK

SEQ ID NO: 10 - protein, 22 aa, Streptococcus pneumoniae
RVFGHKAGMEALQTVTKAANKL

SEQ ID NO: 11 - protein, 6 aa, Streptococcus pneumoniae
VITVYA

SEQ ID NO: 12 - protein, 24 aa, Streptococcus pneumoniae
DQEVKFIMNLPVEFYDNYVFELHA

SEQ ID NO: 13 - protein, 5 aa, Streptococcus pneumoniae
KIQMI

SEQ ID NO: 14 - protein, 14 aa, Streptococcus pneumoniae
KQTFEALTKAEELT

SEQ ID NO: 15 - protein, 6 aa, Streptococcus pneumoniae
ILNFAL

SEQ ID NO: 16 - protein, 17 aa, Streptococcus pneumoniae
GRAEITQALKLISQDVL

SEQ ID NO: 17 - protein, 5 aa, Streptococcus pneumoniae
EELIG

SEQ ID NO: 18 - protein, 4 aa, Streptococcus pneumoniae
FTQH

SEQ ID NO: 19 - protein, 4 aa, Streptococcus pneumoniae
LIIR

SEQ ID NO: 20 - protein, 4 aa, Streptococcus pneumoniae
ELYF

SEQ ID NO: 21 - protein, 12 aa, Streptococcus pneumoniae
EAALQEAILAYN

SEQ ID NO: 22 - protein, 18 aa, Streptococcus pneumoniae
MHHHHHHSSGLVPRGSMA

(19) United States
(12) Patent Application Publication
(10) Pub. No.: US 2004/0191271 A1
(43) Pub. Date: Sep. 30, 2004

(54) Crystal structures of streptococcus undecaprenyl pyrophosphate synthase and uses thereof

(75) Inventors: Kimberly F. Fennell, Niantic, CT (US); Mahmoud N. Mansour, Groton, CT (US); Xiayang Qiu, Mystic, CT (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/815,402

(22) Filed: Mar. 31, 2004

Related U.S. Application Data
(60) Provisional application No. 60/459,053, filed on Mar. 31, 2003.

(51) Int. Cl. A61K 39/02; C12N 9/16

(52) U.S. Cl. 424/190.1; 435/196

(57) ABSTRACT

The invention is directed generally to the structure of prenyltransferases, particularly undecaprenyl pyrophosphate synthase, an enzyme important in bacterial cell wall synthesis. The invention relates to the crystal structure of undecaprenyl pyrophosphate synthase from Streptococcus pneumoniae and its interaction with cofactors and ligands. The invention also relates to the structure of ligand and cofactor binding sites of undecaprenyl pyrophosphate synthase.

The application claims the benefit of U.S. Provisional Application No. 60/459,053, filed on Mar. 31, 2003 and incorporated herein by reference in it is entirety.

### FIELD OF THE INVENTION

The invention is directed generally to the crystal structure of enzymes. More particularly, the invention relates to the atomic structure of enzymes involved in the chain elongation of isoprenoid chains and the use of the structure in drug design.

### BACKGROUND OF THE INVENTION

Prenyltransferases are enzymes important in lipid, peptidoglycan, and glycoprotein biosynthesis. These enzymes act on molecules having a five-carbon isoprenoid substrate. Prenyltransferases are classified into two major subgroups according to whether they catalyze the cis- or trans-isomerization of products in the prenyl chain elongation. E-type prenyltransferases catalyze trans-isomerization and z-type prenyltransferases catalyze cis-isomerization. Unlike the trans-type prenyltransferases, the cis-prenyltransferases are poorly categorized. In particular, little is known about the detailed molecular structure of the active site of cis-prenyltransferases. In consequence, inhibitors of the cis-prenyltransferases have been difficult to establish using a structure-based approach. This deficiency is particularly important because cis-prenyltransferases are involved in the biosynthesis of peptidoglycan in prokaryotes and that of glycoproteins in eukaryotes. Such pathways are crucial for survival of the organism.

Bacterial undecaprenyl pyrophosphate synthase (UPS), also known as undecaprenyl diphosphate synthase, is a z-type prenyltransferase that catalyzes the sequential condensation of eight molecules of isoprenyl pyrophosphate (IPP) with trans, trans-farnesyl pyrophosphate (FPP) to produce the 55-carbon molecule termed undecaprenyl pyrophosphate. Undecaprenyl pyrophosphate is released from the synthase and dephosphorylated to form undecaprenyl phosphate that serves as the essential carbohydrate and lipid carrier in bacterial cell wall and lipopolysaccharide biosynthesis. Undecaprenyl pyrophosphate synthase differs from other members of the prenyltransferase family in the product stereochemistry and product chain length.

Emerging resistance to currently used antibacterial agents has generated an urgent need for antibiotics acting by different mechanisms. Undecaprenyl pyrophosphate synthase exists ubiquitously in bacteria and plays an essential and critical role in the cell wall biosynthesis pathway. Thus, undecaprenyl pyrophosphate synthase is essential for cell viability and provides a valid and unexploited molecular target for antibacterial drug discovery. In consequence, a structure-based approach to development of inhibitors could provide novel antibiotics.

The atomic coordinates for a crystal of undecaprenyl pyrophosphate synthase from M. luteus, in the absence of substrate or cofactor, have been shown. Fujihashi et al., PNAS, 98:4337 (2001). The atomic coordinates are incomplete and some amino acid residues are not defined in the crystal structure.

Similarly, the atomic coordinates for a crystal of undecaprenyl pyrophosphate synthase from E. coli, in the absence of substrate or cofactor, have been shown. Ko et al., J. Biol. Chem., 276:47474 (2001). The atomic coordinates are incomplete and some amino acid residues are not defined in the crystal structure.

### SUMMARY OF THE INVENTION

The invention relates generally to protein crystal structures and uses thereof in drug design. More particularly, the invention relates to Streptococcus undecaprenyl pyrophosphate synthase in crystalline form. The invention also relates to a composition comprising the synthase in crystalline form. The composition can further comprise at least one ligand.

In one embodiment, the invention comprises compositions comprising a Streptococcus undecaprenyl pyrophosphate synthase in crystalline form, the synthase comprising an amino acid sequence at least about 80% homologous to SEQ ID NO:1. In a preferred embodiment of the synthase the amino acid sequence is at least about 90% homologous to SEQ ID NO:1.

The synthase can have a first ligand binding site, a second ligand binding site, or both. Moreover, the composition comprising the synthase can comprise at least one ligand. The ligand can be co-crystallized with the synthase. Suitable ligands include, but are not limited to, farnesyl pyrophosphate, (S)-farnesyl thiopyrophosphate, isoprenyl pyrophosphate, magnesium ion, and sulfate ion. Preferably farnesyl pyrophosphate or (S)-farnesyl thiopyrophosphate are associated with the first ligand binding site and isoprenyl pyrophosphate or sulfate are associated with the second ligand binding site.

In another aspect the undecaprenyl pyrophosphate synthase comprises a first ligand binding site defined by at least one amino acid residue selected from the group consisting of Asp²⁸, Gly²⁹, Gly³¹, Arg³², Arg⁴¹, Ala⁷¹, Arg⁷⁹, Leu⁹⁰, Pro⁹¹, and Phe¹⁴³. In a preferred embodiment, the crystal can comprise a first ligand binding site defined by amino acid residues 28, 29, 31, 32, 41, 71, 91, and 143 having atoms having atomic coordinates according to FIG. 2.

In yet another aspect, the undecaprenyl pyrophosphate synthase comprises a second binding site formed by at least one amino acid residue selected from the group consisting of Asp²⁸, Arg²⁰⁰, Arg²⁰⁶, and Ser²⁰⁸ from one chain (A) of the dimer, and Glu²¹⁹ and either Gly²⁵⁰ or Gly²⁵¹ from the other chain (B) of the dimer. Thus, both polypeptide chains can contribute to the second binding site. In a preferred embodiment, the crystal can comprise a second ligand binding site defined by amino acid residues 28, 200, 206, 208, and 219(B) having atoms having atomic coordinates according to FIG. 2.

The invention also relates to undecaprenyl pyrophosphate synthase in crystalline form wherein the synthase is S. pneumoniae undecaprenyl pyrophosphate synthase.

In still another aspect, the invention is directed to compositions comprising undecaprenyl pyrophosphate synthase in crystalline form as well as a ligand therefor. The synthase can be from any organism, not limited to Streptococcus.

One aspect of the invention is directed to methods of designing or identifying a potential ligand for an undecaprenyl pyrophosphate synthase comprising using a three-dimensional structure of an undecaprenyl pyrophosphate synthase, employing the three dimensional structure to design or select the potential ligand, obtaining the potential ligand; and contacting the potential ligand with the undecaprenyl pyrophosphate synthase to determine binding to the undecaprenyl pyrophosphate synthase. One skilled in the art will recognize that the steps of the method can be carried out in various orders. The three-dimensional structure of a binding site can be defined by atomic coordinates of amino acid residues 28, 29, 31, 32, 41, 71, 91, and 143 according to FIG. 2.

In an embodiment, the methods can further comprise identifying chemical entities or fragments thereof, capable of binding to the undecaprenyl pyrophosphate synthase; and assembling the identified chemical entities or fragments thereof into a single molecule to provide the structure of the potential ligand.

The potential ligand can be an inhibitor. In one embodiment the inhibitor is a competitive inhibitor. In another embodiment the inhibitor is a non-competitive inhibitor. The ligand can be designed de novo. Alternatively, the ligand can be designed from a known inhibitor. The method can further comprise using the atomic coordinates according to FIG. 2, or portion thereof, of a ligand bound to the undecaprenyl pyrophosphate synthase.

Another aspect of the invention is directed to methods for identifying a potential inhibitor of a mutant undecaprenyl pyrophosphate synthase, the method comprising using a three-dimensional structure of undecaprenyl pyrophosphate synthase as defined by atomic coordinates of undecaprenyl pyrophosphate synthase according to FIG. 2; replacing one or more undecaprenyl pyrophosphate synthase amino acids selected from 28, 29, 31, 32, 41, 71, 79, 90, 91, 143, 200, 206, 208, 219 and either 250 or 251 of SEQ ID NO:1 in the three-dimensional structure with a different naturally occurring amino acid, thereby forming a mutant undecaprenyl pyrophosphate synthase; employing the three-dimensional structure to design or select the potential inhibitor; synthesizing the potential inhibitor; and contacting the potential inhibitor with the mutant undecaprenyl pyrophosphate synthase or the undecaprenyl pyrophosphate synthase in the presence of a substrate to test the ability of the potential inhibitor to inhibit the undecaprenyl pyrophosphate synthase or the mutant undecaprenyl pyrophosphate synthase. The potential inhibitor can be selected from a database.

In another aspect, the invention is directed to methods for identifying a potential inhibitor for an undecaprenyl pyrophosphate synthase, comprising using a three-dimensional structure of the synthase as defined by atomic coordinates of undecaprenyl pyrophosphate synthase according to FIG. 2; employing said three-dimensional structure to design or select the potential inhibitor; synthesizing the potential inhibitor; and contacting the potential inhibitor with the synthase in the presence of a substrate to determine the ability of the potential inhibitor to inhibit the synthase.

In one embodiment, the three-dimensional structure can be further defined by atomic coordinates of amino acid residues 200, 206, and 208 according to FIG. 2. In another embodiment, the three-dimensional structure can be further defined by atomic coordinates of amino acid residue 219(B) according to FIG. 2. Amino acid residues labeled “B” are from the complementary polypeptide chain of the dimer.

The potential ligand can be designed to form a hydrogen bond with at least one amino acid residue selected from the group consisting of Gly²⁹, Gly³¹, Arg³², Arg⁴¹, and Arg⁷⁹. In addition, or alternatively, the potential ligand can be designed to form a hydrogen bond with at least one amino acid residue selected from the group consisting of Arg²⁰⁰, Arg²⁰⁶, Ser²⁰⁸, Glu²¹⁹(B), and either Gly²⁵⁰(B) or Gly²⁵¹(B). In another embodiment, the potential ligand can be designed to form a hydrophobic bond with at least one amino acid residue selected from the group consisting of Ala⁷¹, Leu⁹⁰, Pro⁹¹, and Phe¹⁴³.

In one aspect the invention is directed to ligands identified by these methods.

The invention also relates to methods of identifying a ligand capable of binding to an undecaprenyl pyrophosphate synthase substrate binding site, comprising: (a) introducing into a suitable computer program information defining the binding site comprising first atomic coordinates of amino acids capable of binding to a synthase substrate, wherein the program displays the three-dimensional structure of the binding site; (b) creating a three dimensional model of a test compound in the computer program; (c) docking the model of the test compound to the structure of the binding site; (d) creating a second three dimensional model of the substrate or an inhibitor of the synthase and docking the second model thereto; and (e) comparing the docking of the test compound and of the substrate or an inhibitor of the synthase to provide an output of the program. In one embodiment, the method further comprises introducing into the computer program second atomic coordinates of water molecules bound to the substrate. In another embodiment, the method further comprises introducing into the computer program third atomic coordinates of at least one synthase structural element selected from the group consisting of an alpha helix, a 3₁₀ helix, a strand of beta sheet, and a coil.

In yet another embodiment the methods further comprise: (f) incorporating the test compound into a biological or biochemical assay for synthase activity; and (g) determining whether the test compound inhibits synthase activity in the assay.

The invention is also directed to methods of drug design comprising using the atomic coordinates of an S. pneumoniae undecaprenyl pyrophosphate synthase, or substantial portion thereof, having at least one ligand binding site, to computationally evaluate relative associations of chemical entities with the ligand binding site. The chemical entity can be an intermediate in a farnesyl pyrophosphate elongation reaction, or an analog thereof.

In another aspect the invention is directed to methods for solving a crystal form comprising using the atomic coordinates of S. pneumoniae undecaprenyl pyrophosphate synthase crystal, or portions thereof, to solve a crystal form of a mutant, homolog or co-complex of the undecaprenyl pyrophosphate synthase by molecular replacement. The method can further comprise using the atomic coordinates of a ligand bound to undecaprenyl pyrophosphate synthase.

One aspect of the invention is directed to machine-readable data storage media comprising a data storage material encoded with machine-readable data comprising atomic coordinates comprising amino acid residues 28, 29, 31, 32, 41, 71, 91, and 143 according to FIG. 2. In one embodiment, the machine-readable data further comprise atomic coordinates comprising at least one amino acid residue selected from the group consisting of 200, 206, 208, and 219(B) according to FIG. 2. In another embodiment, the machine-readable data comprise the three-dimensional structure of S. pneumoniae undecaprenyl pyrophosphate synthase.

In another aspect, the invention comprises computer-implemented tools for design of a drug, comprising: (a) a three-dimensional structure of a undecaprenyl pyrophosphate synthase as defined by atomic coordinates of a S. pneumoniae undecaprenyl pyrophosphate synthase having at least one ligand binding site; (b) a model of a chemical entity; and (c) a computer program addressing the coordinates and capable of modeling the chemical entity in the ligand binding site to produce an output.

In yet another aspect, the invention comprises computers for producing a three-dimensional representation of a undecaprenyl pyrophosphate synthase ligand binding site comprising: (a) a machine-readable data storage medium comprising a data storage material encoded with machine-readable data comprising the atomic coordinates comprising the amino acid residues 28, 29, 31, 32, 41, 71, 91, and 143 according to FIG. 2; (b) a working memory for storing instructions for processing the machine-readable data; (c) a central-processing unit coupled to the working memory and to the machine-readable data storage medium for processing the machine readable data into the three-dimensional representation; and (d) a display coupled to the central-processing unit for displaying the three-dimensional representation. The computer can also produce a three-dimensional representation of the ligand binding site of an undecaprenyl pyrophosphate synthase; and the machine-readable data can comprise the atomic coordinates of the ligand binding site.

### BRIEF DESCRIPTION OF THE FIGURES

FIG. 1 is a diagram of the crystal structure of S. pneumoniae undecaprenyl pyrophosphate synthase dimer.

FIG. 2 shows the atomic coordinates of the (A) and (B) polypeptide chains of S. pneumoniae undecaprenyl pyrophosphate synthase. The number of the amino acid residue, as it compares with SEQ ID NO:1, is found in the 6^{th} column reading left to right.

### DETAILED DESCRIPTION OF THE INVENTION

In order that the invention described herein be fully understood, the following detailed description is set forth. The following table lists the amino acid abbreviations used herein.

| | |
|---|---|
| A = Ala = Alanine | T = Thr = Threonine |
| V = Val = Valine | C = Cys = Cysteine |
| L = Leu = Leucine | Y = Tyr = Tyrosine |
| I = Ile = Isoleucine | N = Asn = Asparagine |
| P = Pro = Proline | Q = Gln = Glutamine |
| F = Phe = Phenylalanine | D = Asp = Aspartic Acid |
| W = Trp = Tryptophan | E = Glu = Glutamic Acid |
| M = Met = Methionine | K = Lys = Lysine |
| G = Gly = Glycine | R = Arg = Arginine |
| S = Ser = Serine | H = His = Histidine |

For convenience, certain terms employed in the specification, examples, and appended claims are collected here. Unless defined otherwise, all technical and scientific terms used herein have the same meaning as commonly understood by one of ordinary skill in the art to which this invention belongs.

The term “naturally occurring amino acids” means the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine and lysine. Unless specifically indicated, all amino acids referred to in this application are in the L-form.

The term “unnatural amino acids” means amino acids that are not naturally found in proteins. Examples of unnatural amino acids used herein, include selenocysteine and selenomethionine. In addition, unnatural amino acids include D-phenylalanine and the D or L forms of nor-leucine, para-nitrophenylalanine, homophenylalanine, para-fluorophenylalanine, 3-amino-2-benzylpropionic acid, and homoarginine.

The term “positively charged amino acid” includes any naturally occurring or unnatural amino acid having a positively charged side chain under normal physiological conditions. Examples of positively charged naturally occurring amino acids are arginine, lysine and histidine.

The term “negatively charged amino acid” includes any naturally occurring or unnatural amino acid having a negatively charged side chain under normal physiological conditions. Examples of negatively charged naturally occurring amino acids are aspartic acid and glutamic acid.

The term “hydrophobic amino acid” means any amino acid having an uncharged, nonpolar side chain that is relatively insoluble in water. Examples of naturally occurring hydrophobic amino acids are alanine, leucine, isoleucine, valine, proline, phenylalanine, tryptophan and methionine. Histidine and tyrosine can also participate in hydrophobic bonds.

The term “hydrophilic amino acid” means any amino acid having an uncharged, polar side chain that is relatively soluble in water. Examples of naturally occurring hydrophilic amino acids are serine, threonine, tyrosine, asparagine, glutamine, and cysteine.

The term “hydrogen bond” is used to describe an interaction between polar atoms including N, O, and S, in which hydrogen forms a bridge. The side chains of ionic and hydrophilic amino acids and of amide moieties in the peptide backbone are candidates for hydrogen bonds. Polar and ionic moieties in substrates and inhibitors are candidates for hydrogen bonding.

The term “hydrophobic bond” is used to describe a Van der Waals interaction of non-polar moieties that are enthalpicly or entropicly favored over interaction with water or polar groups. Thus, one model for hydrophobic bonds is the gain in free energy formed by exclusion of water. Prime candidates for forming hydrophobic bonds are the aliphatic tail of farnesyl pyrophosphate and side chains of amino acid residues including phenylalanine, tryptophan, proline, leucine, isoleucine, valine, alanine, histidine, and tyrosine.

The term “residue” in amino acid residue refers to the part of an amino acid incorporated into a polypeptide.

The term “ligand” refers to a chemical entity that binds to, or associates with, a synthase. Often, but not always, a ligand is a small molecule. A substrate is a ligand that can be, under appropriate conditions, chemically acted upon by the synthase. In particular, farnesyl pyrophosphate is a substrate that binds to the synthase in the presence of magnesium ion, acting as a cofactor, but does not undergo a chemical reaction unless a second substrate, that is isoprenyl pyrophosphate, is present, and other conditions necessary for catalysis are met.

The term “mutant” refers to an undecaprenyl pyrophosphate synthase polypeptide, i.e. a polypeptide displaying the biological activity of wild-type, undecaprenyl pyrophosphate synthase, characterized by the replacement of at least one amino acid from the wild-type, undecaprenyl pyrophosphate synthase sequence according to SEQ ID NO:1. Such a mutant may be prepared, for example, by expression of undecaprenyl pyrophosphate synthase cDNA previously altered in its coding sequence by oligonucleotide-directed mutagenesis, or other means well-known in the art.

Undecaprenyl pyrophosphate synthase mutants may also be generated, e.g., by site-specific incorporation of unnatural amino acids into undecaprenyl pyrophosphate synthase proteins using the general biosynthetic method of Noren et al., Science, 244:182-88 (1989). In this method, the codon encoding the amino acid of interest in wild-type undecaprenyl pyrophosphate synthase is replaced by a “blank” nonsense codon, TAG, using oligonucleotide-directed mutagenesis. A suppressor tRNA directed against this codon is then chemically aminoacylated in vitro with the desired unnatural amino acid. The aminoacylated tRNA is then added to an in vitro translation system to yield a mutant undecaprenyl pyrophosphate synthase enzyme with the site-specific incorporated unnatural amino acid.

Selenocysteine or selenomethionine may be incorporated into wild-type or mutant undecaprenyl pyrophosphate synthase as described below. In this method, the wild-type or mutagenized undecaprenyl pyrophosphate synthase cDNA may be expressed in a host organism on a growth medium depleted of either natural cysteine or methionine (or both) but enriched in selenocysteine or selenomethionine (or both).

Altered surface charge describes a change in one or more of the charge units of a mutant polypeptide, at physiological pH, as compared to wild-type undecaprenyl pyrophosphate synthase. This is preferably achieved by mutation of at least one amino acid of wild-type undecaprenyl pyrophosphate synthase to an amino acid comprising a side chain with a different charge at physiological pH than the original wild-type side chain.

The change in surface charge is determined by measuring the isoelectric point (pI) of the polypeptide molecule containing the substituted amino acid and comparing it to the isoelectric point of the wild-type undecaprenyl pyrophosphate synthase molecule.

Altered substrate specificity refers to a change in the ability of a mutant undecaprenyl pyrophosphate synthase to bind and use analogs of FPP, IPP, or both.

A “competitive” inhibitor is one that inhibits undecaprenyl pyrophosphate synthase activity by binding to the same form of undecaprenyl pyrophosphate synthase as its substrate binds—thus directly competing with the substrate for the active site of undecaprenyl pyrophosphate synthase. Competitive inhibition can be reversed completely by sufficiently increasing the substrate concentration.

An “uncompetitive” inhibitor is one that inhibits undecaprenyl pyrophosphate synthase by binding to a different form of the enzyme than does the substrate. Such inhibitors bind to undecaprenyl pyrophosphate synthase already bound with the substrate and not to the free enzyme. Uncompetitive inhibition cannot be reversed completely by increasing the substrate concentration.

A “non-competitive” inhibitor is one that can bind to either the free or substrate bound form of undecaprenyl pyrophosphate synthase.

Those of skill in the art may identify inhibitors as competitive, uncompetitive or non-competitive by computer fitting enzyme kinetic data using standard equations according to, e.g., Segel, I. H., Enzyme Kinetics, J. Wiley & Sons, (1975).

The term “homologue” as used herein means a protein, polypeptide, oligopeptide, or portion thereof, having preferably at least 80%, more preferably at least 90%, amino acid sequence identity with Streptococcus undecaprenyl pyrophosphate synthase or any functional or structural domain of undecaprenyl pyrophosphate synthase.

The term “co-complex” means undecaprenyl pyrophosphate synthase or a mutant or homologue of undecaprenyl pyrophosphate synthase in covalent or non-covalent association with a chemical entity or compound.

The term “associating with” refers to a condition of proximity between a chemical entity or compound, or portions thereof, and an undecaprenyl pyrophosphate synthase molecule or portions thereof. The association may be non-covalent, wherein the juxtaposition is energetically favored by hydrogen bonding or van der Waals or electrostatic interactions, or it may be covalent.

The terms “beta sheet or β-sheet” refers to the conformation of a polypeptide chain stretched into an extended zig-zag conformation. Portions of polypeptide chains termed strands that run “parallel” all run in the same direction, amino terminus to carboxy terminus. Polypeptide chains or portions thereof, termed strands, that are “antiparallel” run in the opposite directions.

The term “binding site” refers to a region of the synthase comprised of amino acid residues and optionally cofactors to which a ligand can bind. Undecaprenyl pyrophosphate synthase has binding sites for at least farnesyl pyrophosphate and longer chain derivatives of FPP, isoprenyl pyrophosphate, magnesium ion, and sulfate ion.

The term “active site” refers to any or all of the following sites in undecaprenyl pyrophosphate synthase: the FPP binding site, the IPP binding site, the site of the synthase reaction products and intermediates, the magnesium ion site, and the sulfate site. In one particular usage, “active site” refers to the site where the catalytic reaction occurs.

The term “atomic coordinates” refers to mathematical coordinates derived from mathematical equations related to the patterns obtained on diffraction of a monochromatic beam of X-rays by the atoms (scattering centers) of an undecaprenyl pyrophosphate synthase molecule in crystal form. The diffraction data are used to calculate an electron density map of the repeating unit of the crystal. The electron density maps are used to establish the positions of the individual atoms within the unit cell of the crystal. The similar term “structure coordinates” refers to the mathematical coordinates of the individual atoms. It is to be understood that a set of atomic coordinates includes not just the exact coordinates as listed, but any translational or rotational variation in those coordinates, as long as the relative positions of the atoms is maintained.

The term “substantial portion” of atomic coordinates refers to a plurality of at least twelve atomic coordinates that define or partially define the location of several atoms in the synthase or ligand. Preferably, a substantial portion is at least 24 coordinates. More preferably, a substantial portion is at least 36 coordinates. The coordinates can be within the standard deviation.

The term “heavy atom derivatization” refers to a method of producing a chemically modified form of a crystal of undecaprenyl pyrophosphate synthase. In practice, a crystal is soaked in a solution containing heavy metal atom salts, or organometallic compounds, e.g., lead chloride, gold thiomalate, thimerosal or uranyl acetate, which can diffuse through the crystal and bind to the surface of the protein. The location(s) of the bound heavy metal atom(s) can be determined by X-ray diffraction analysis of the soaked crystal. This information, in turn, is used to generate the phase information used to construct three-dimensional structure of the enzyme. See, for example, Blundel, T. L. and N. L. Johnson, Protein Crystallography, Academic Press (1976).

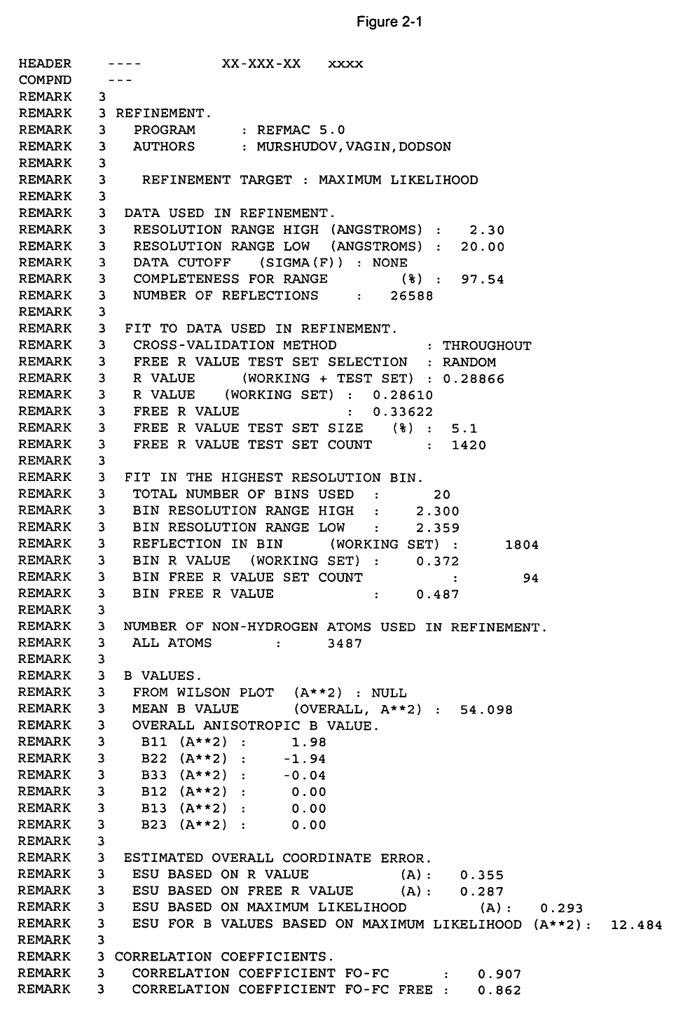

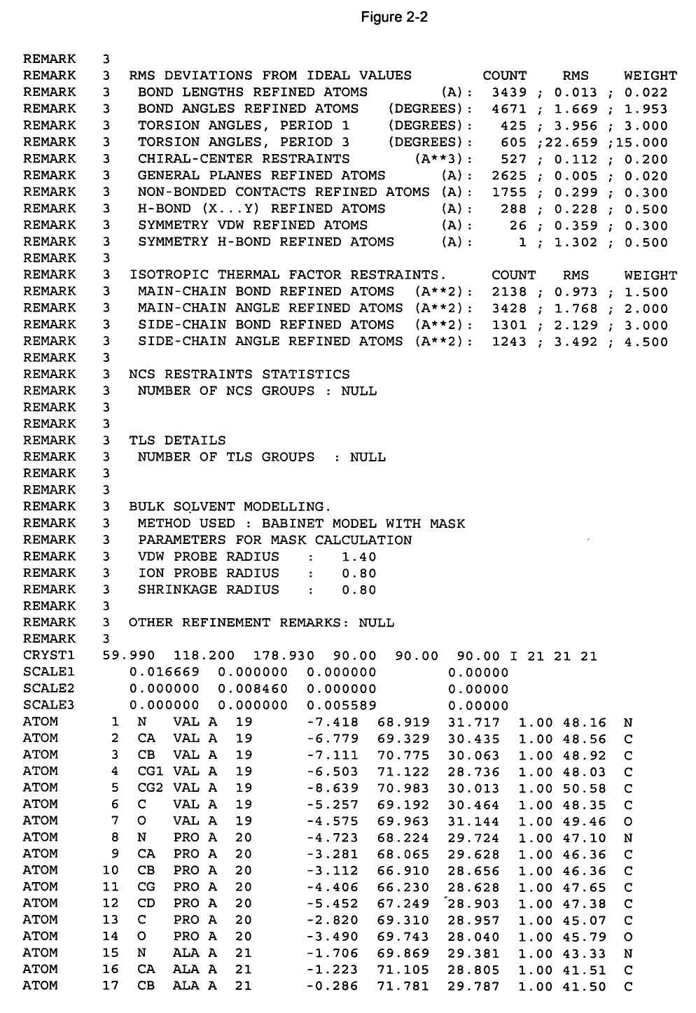

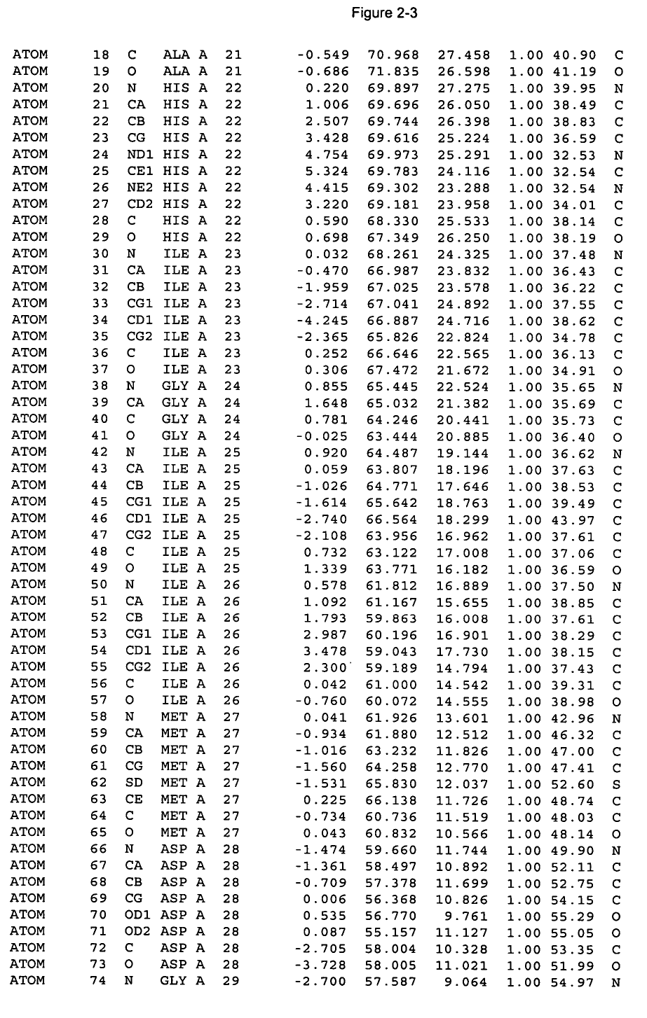

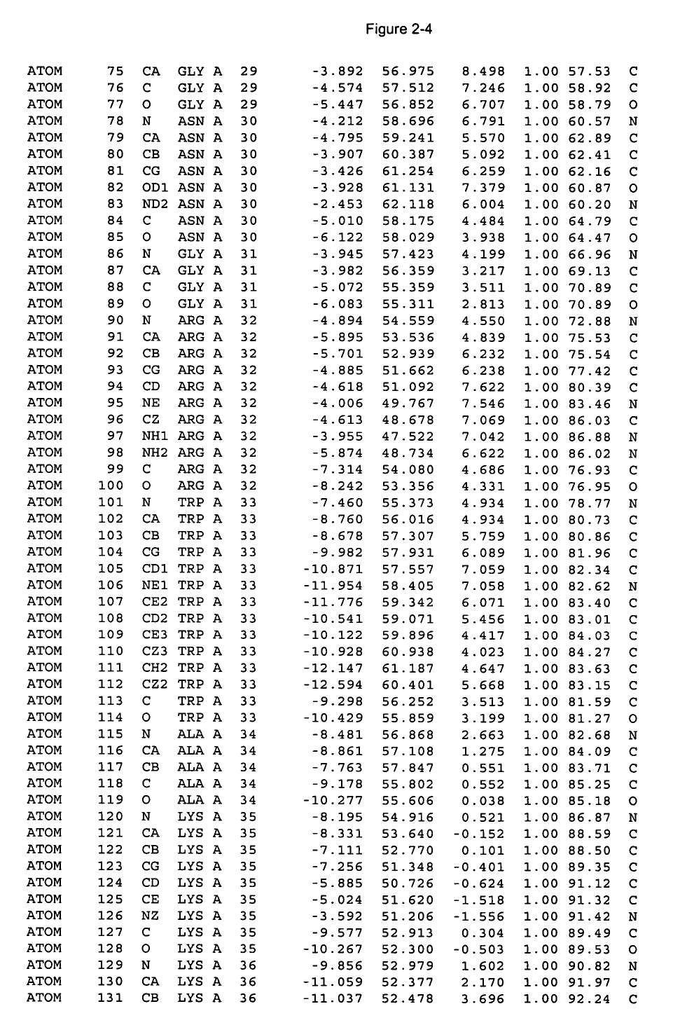

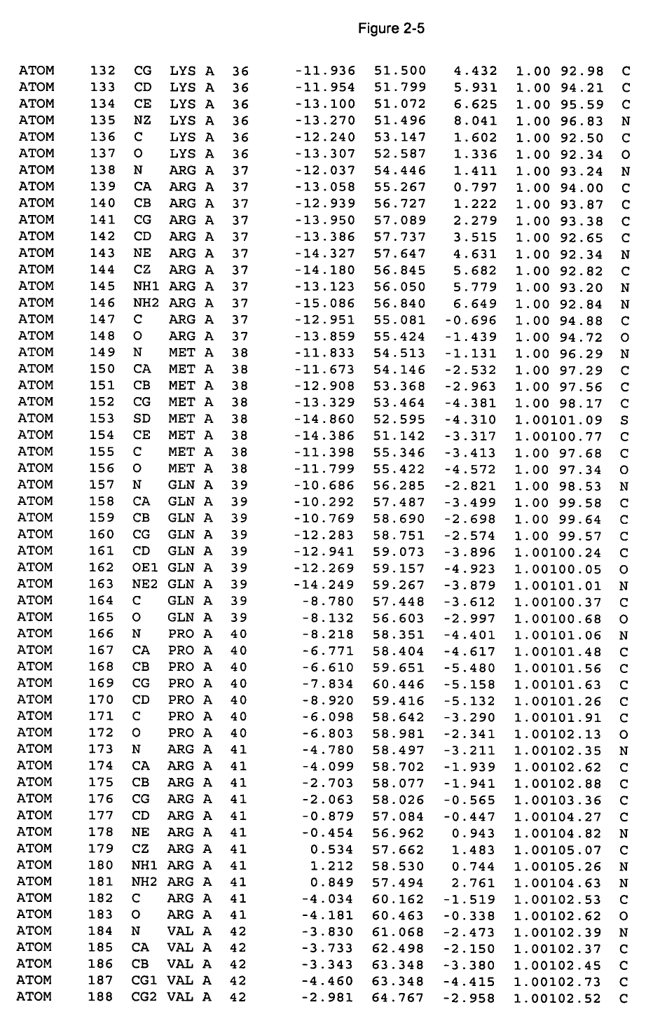

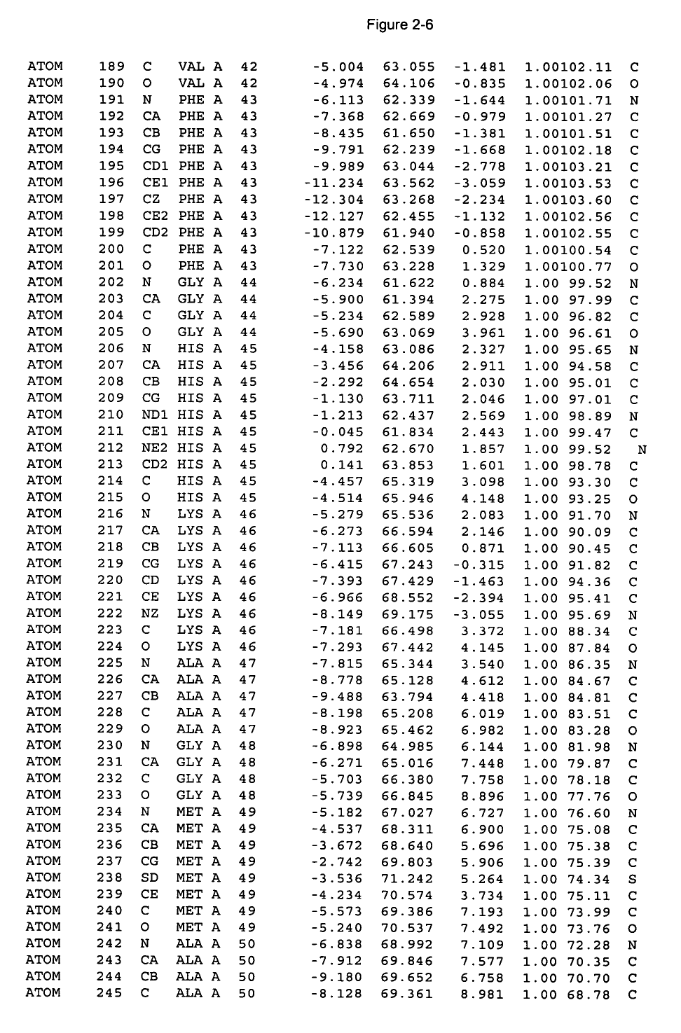

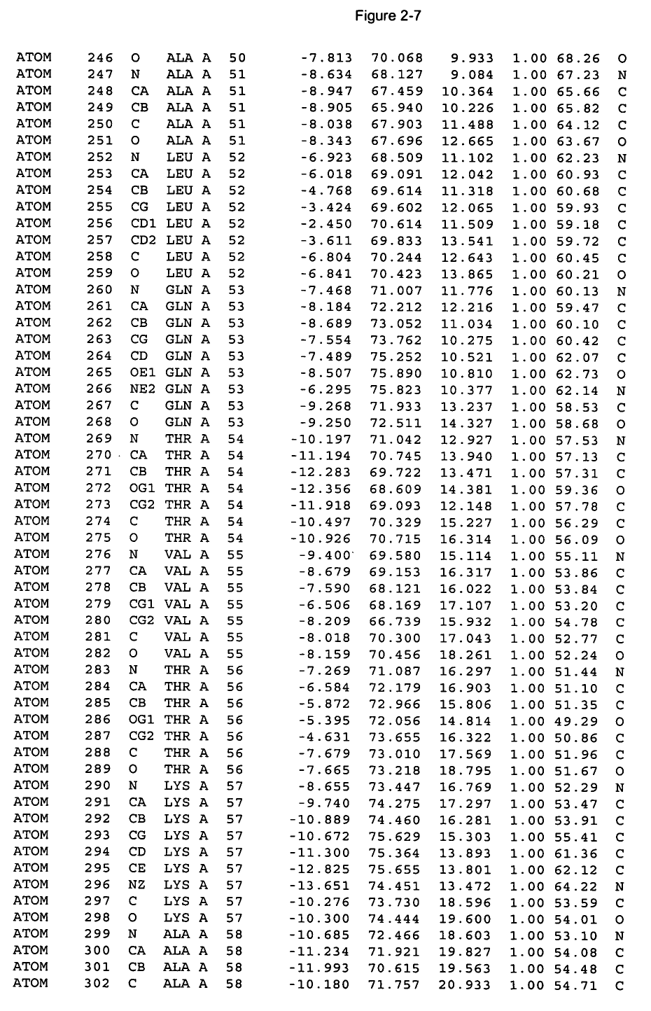

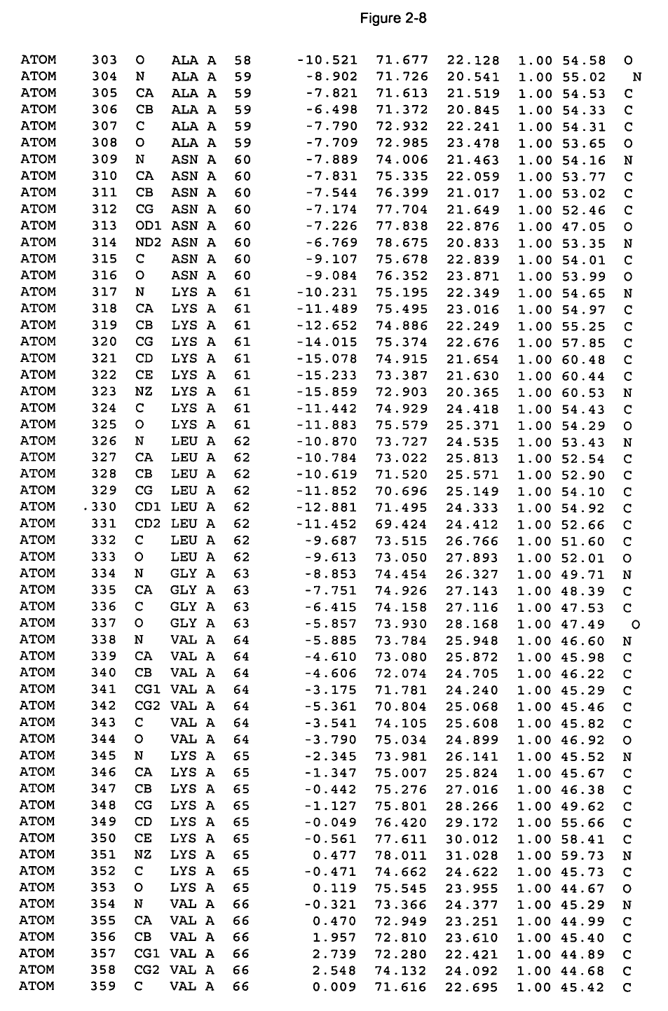

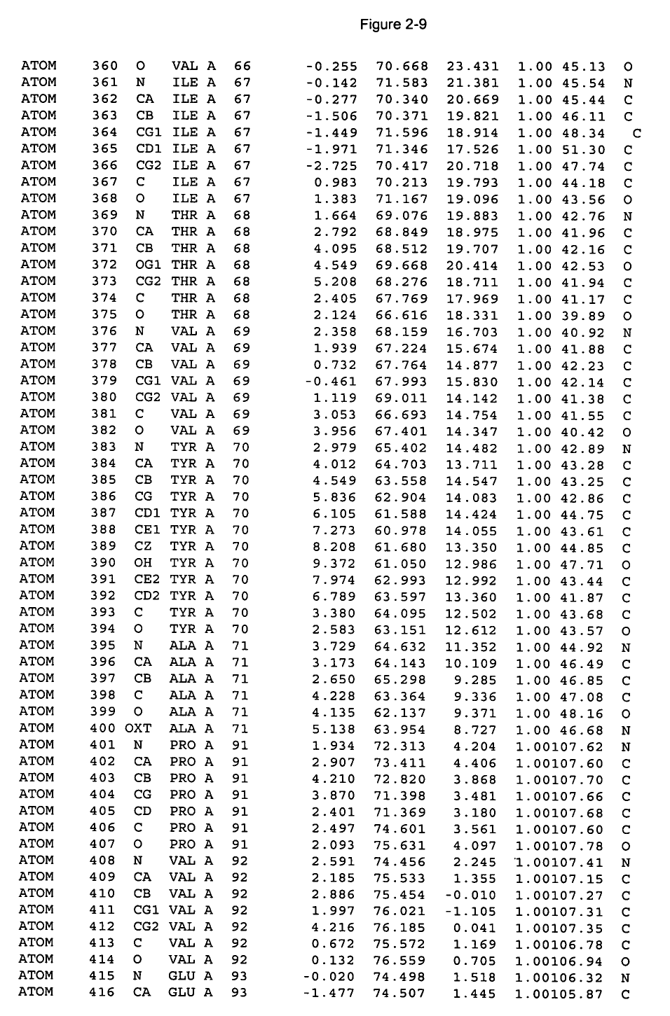

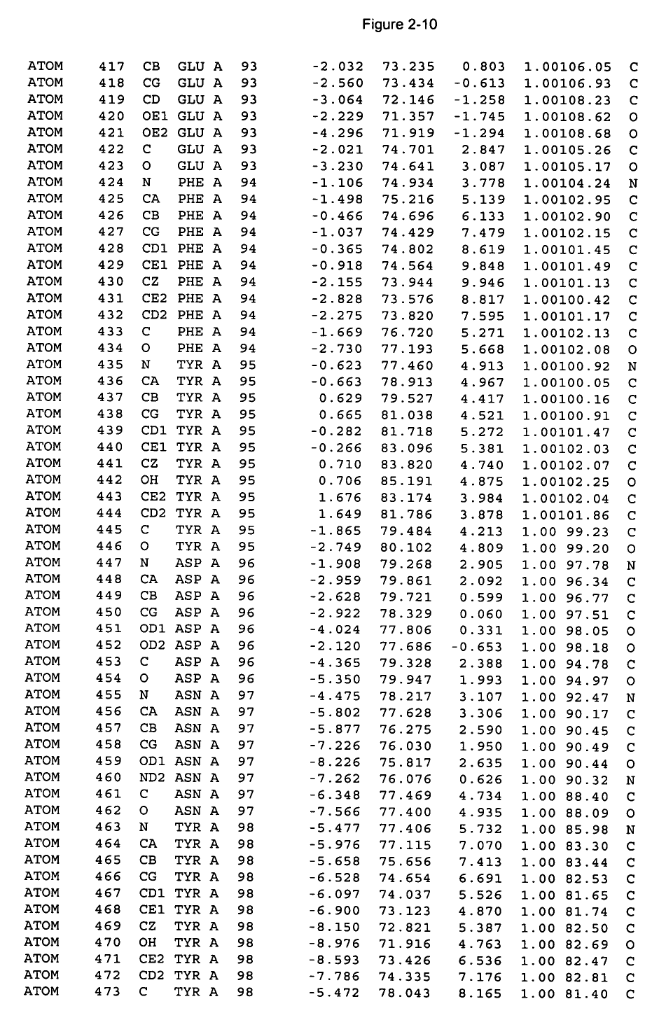

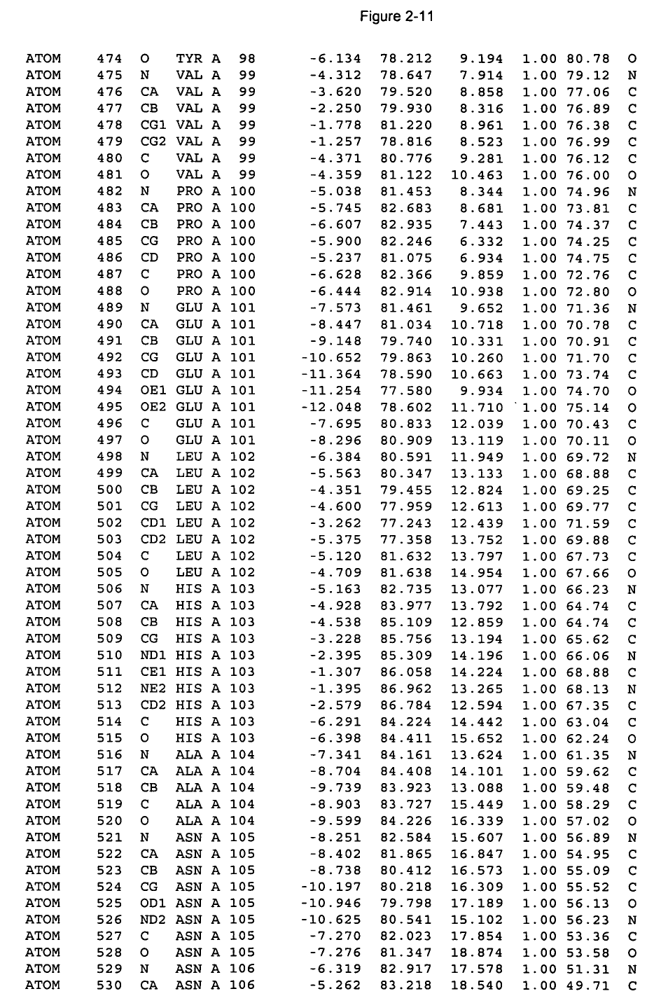

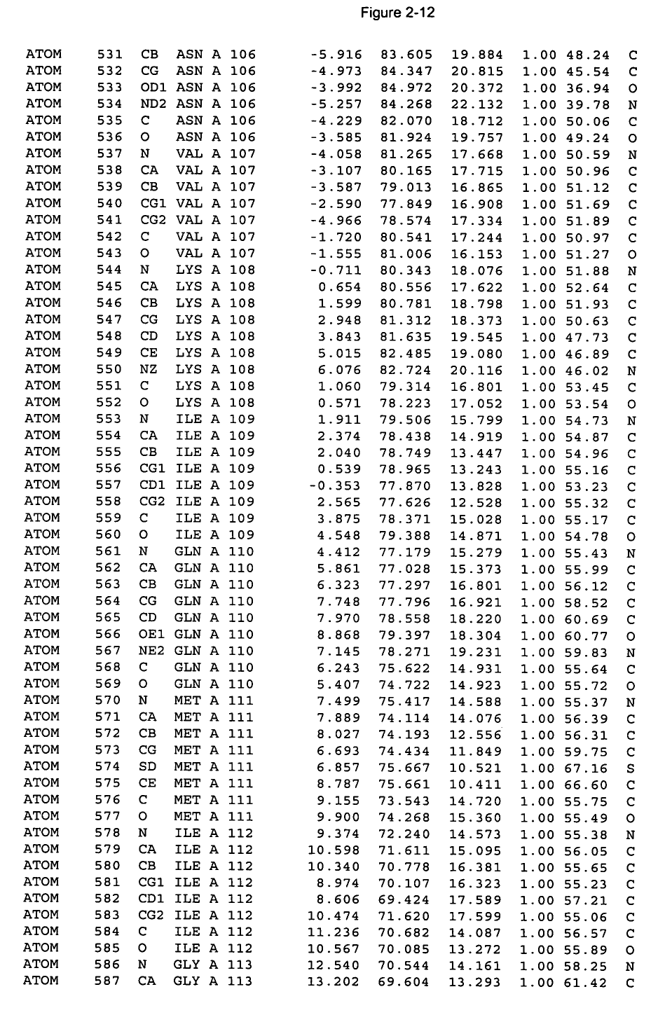

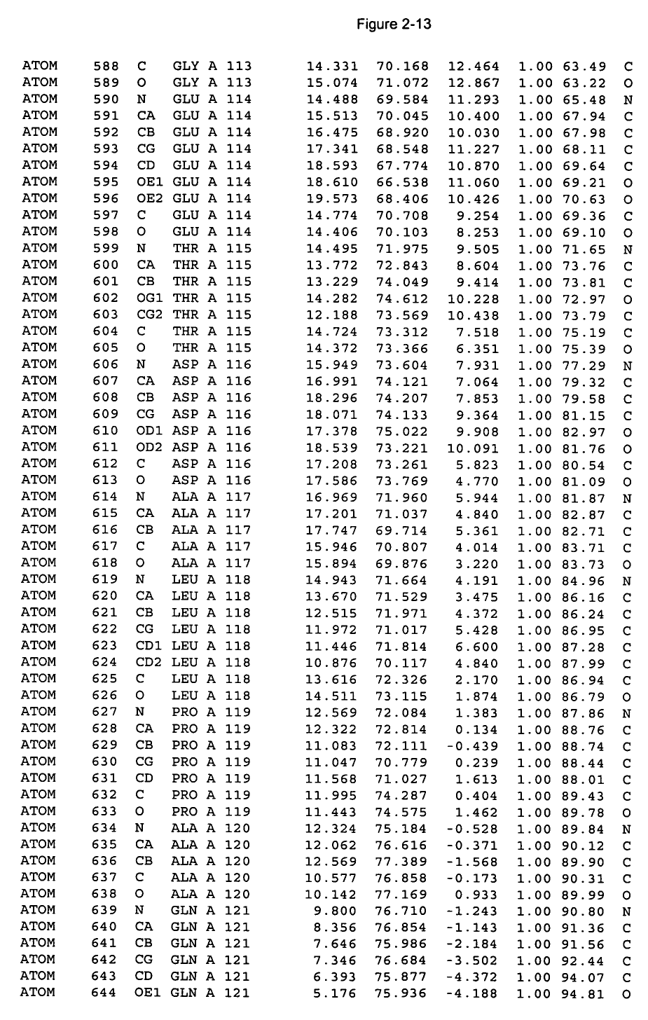

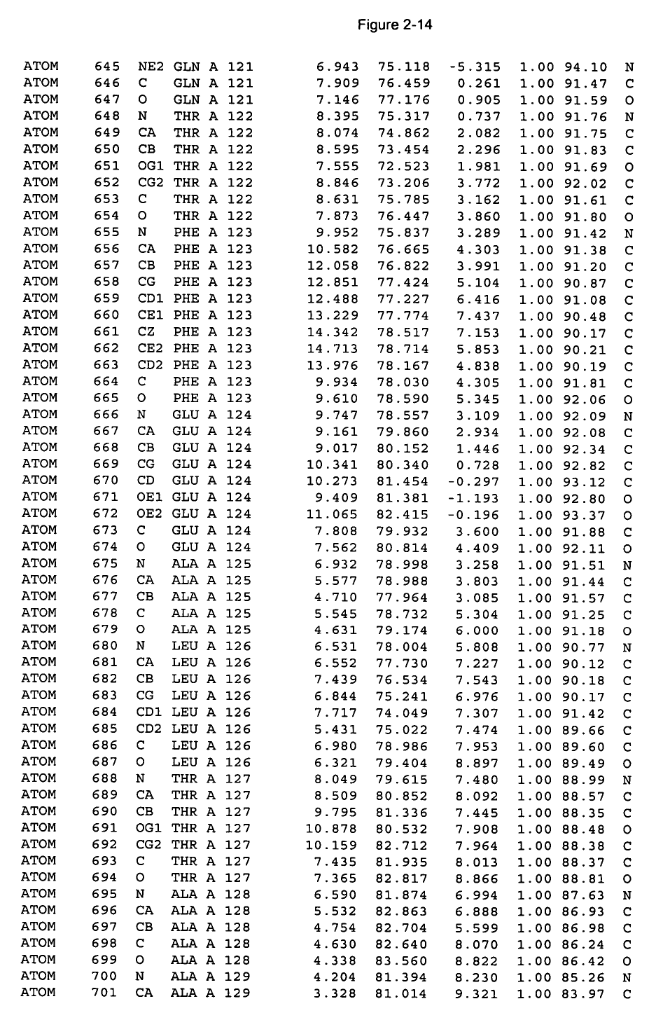

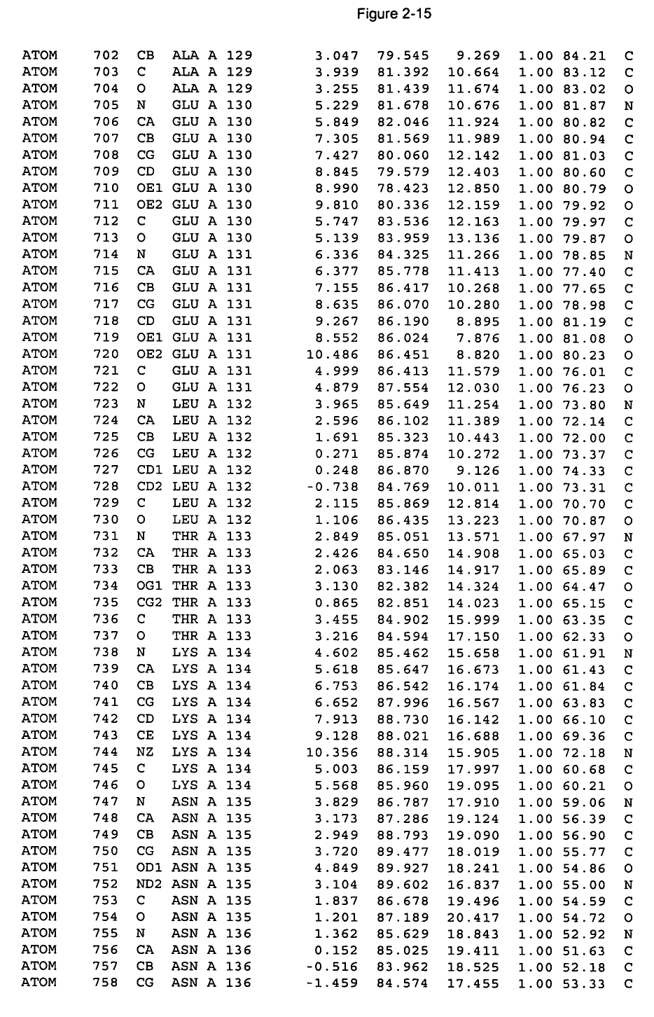

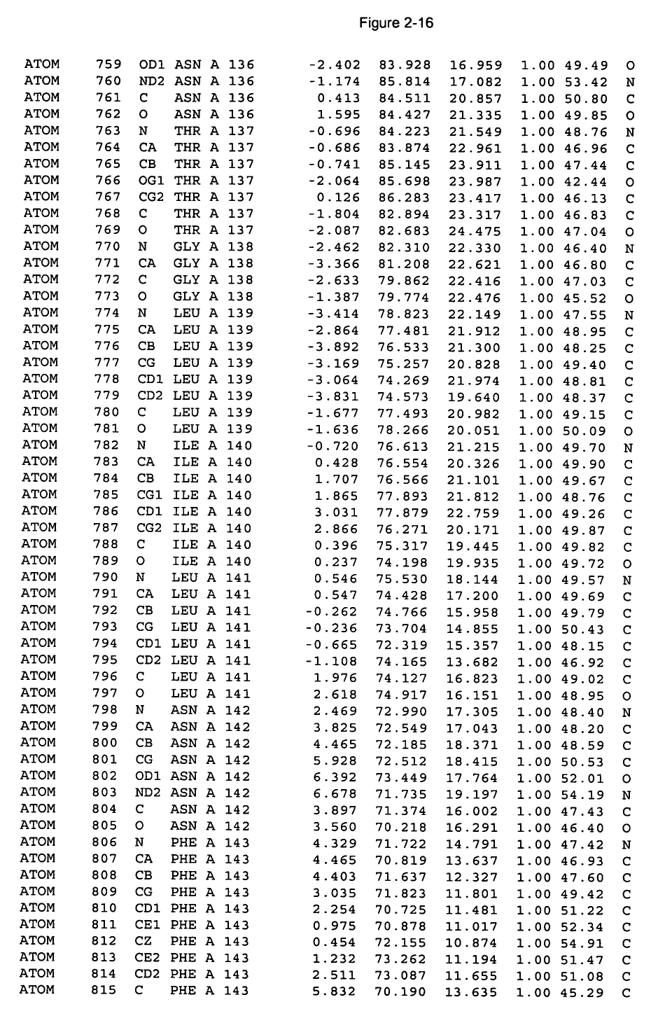

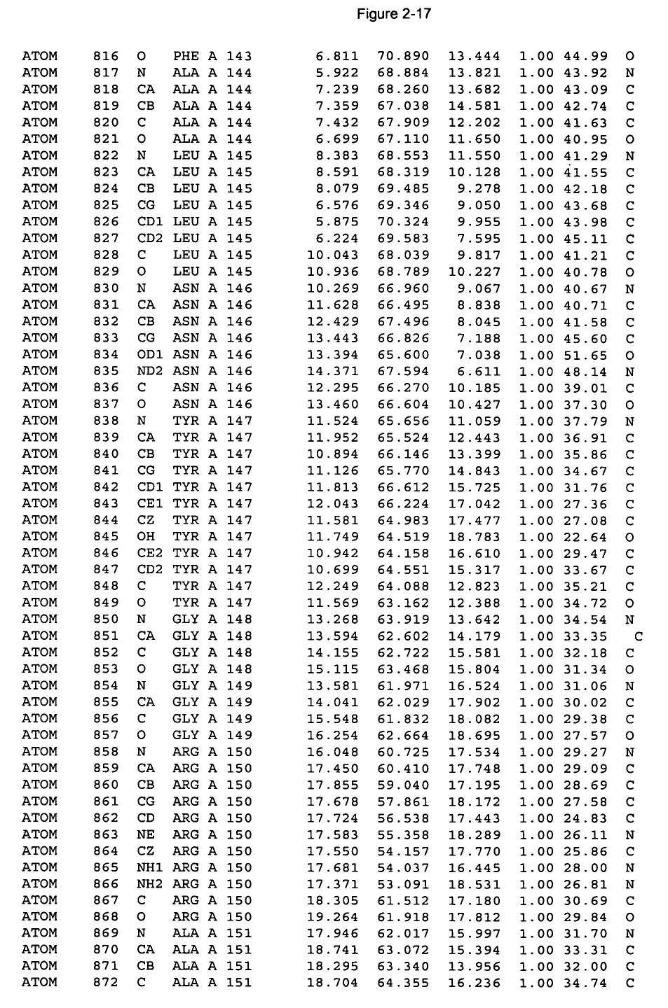

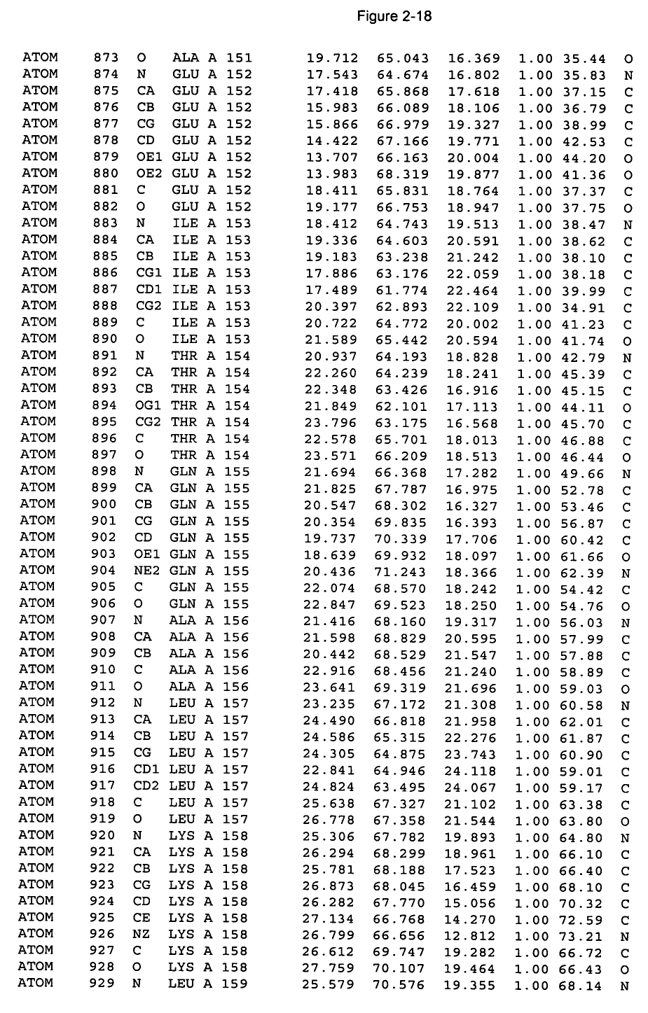

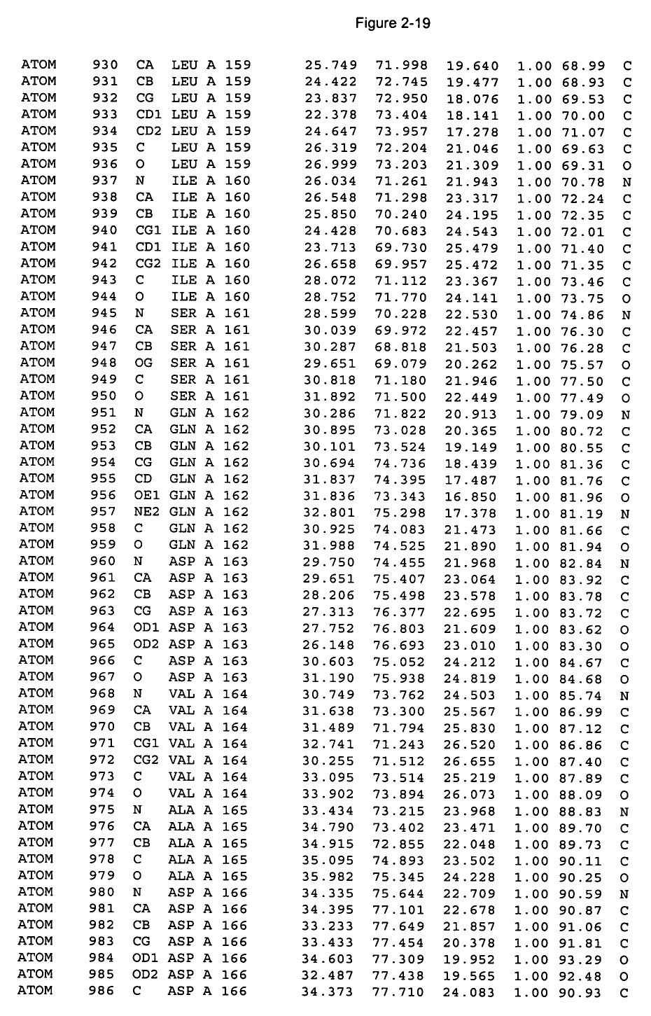

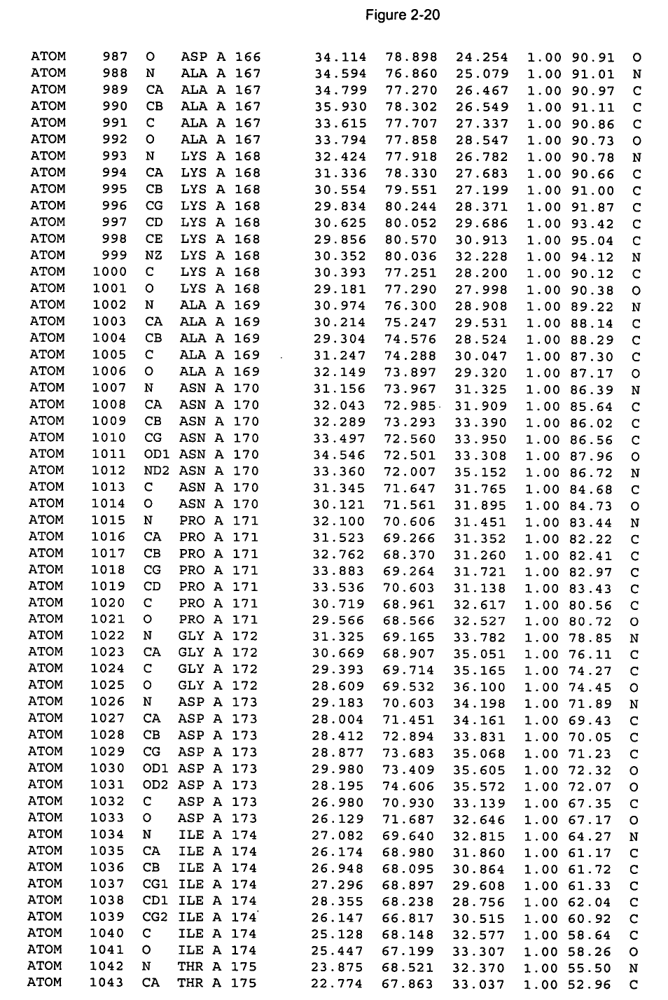

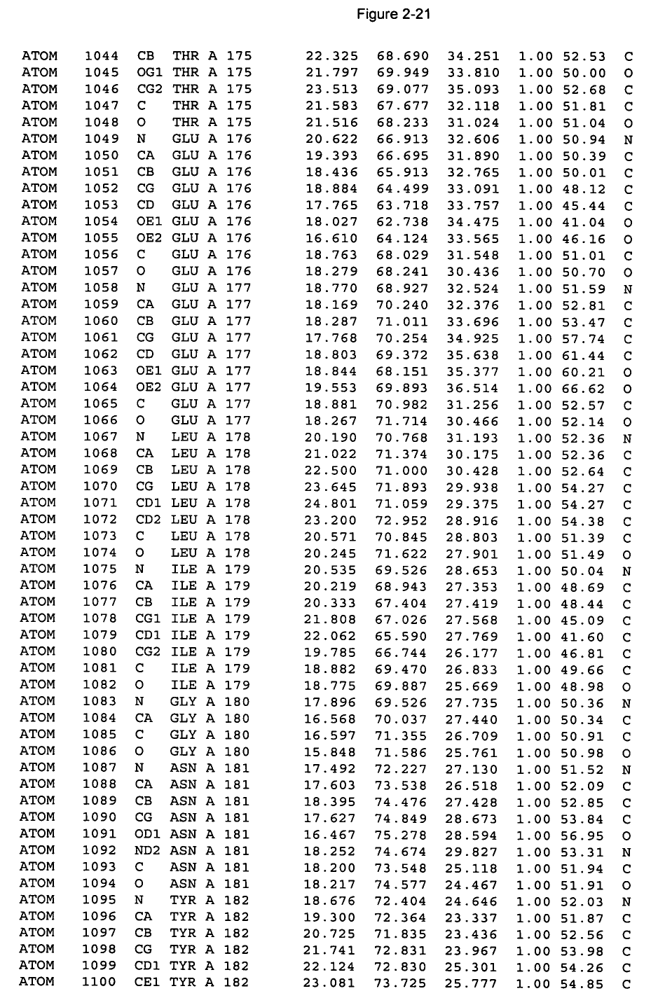

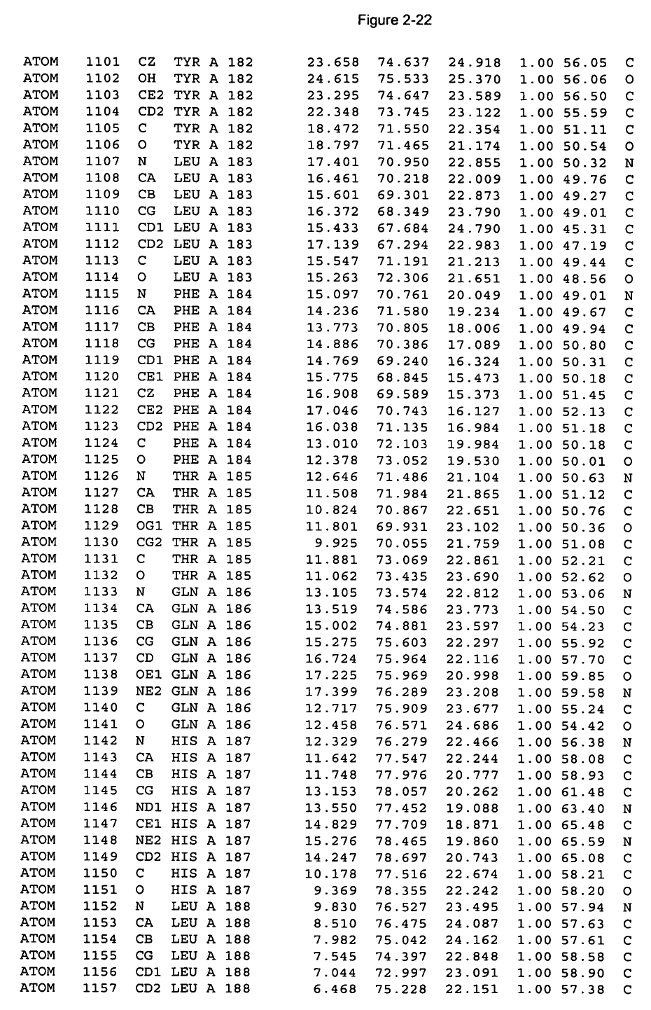

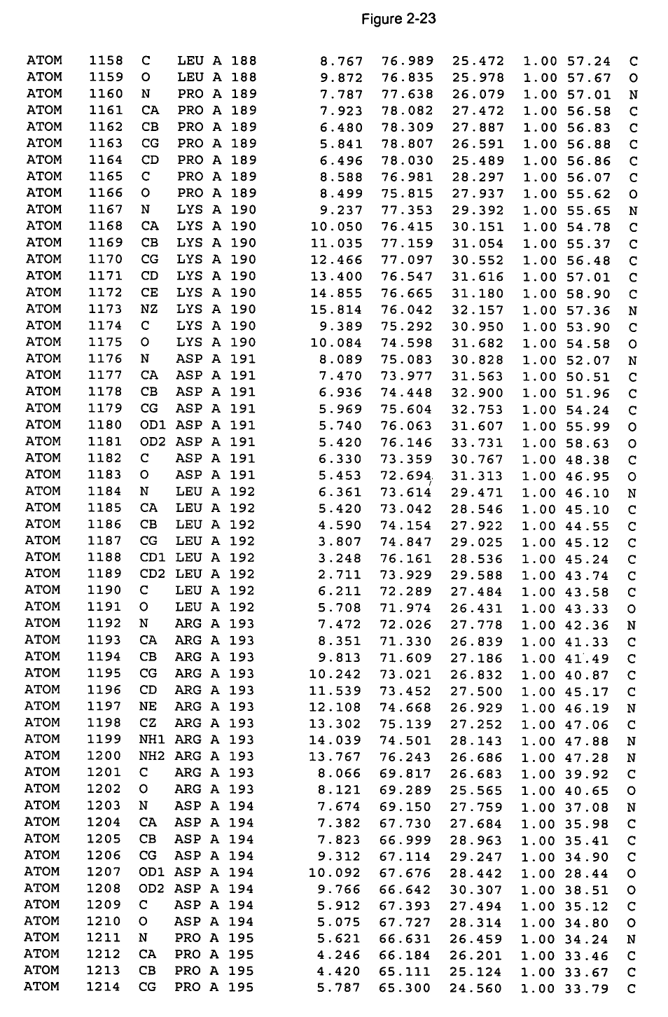

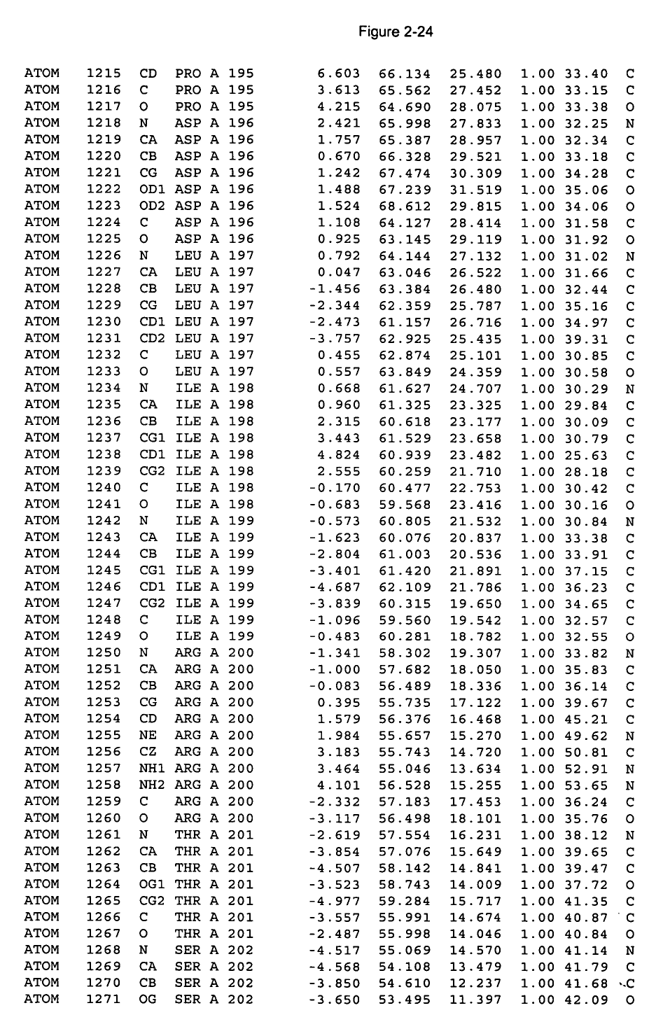

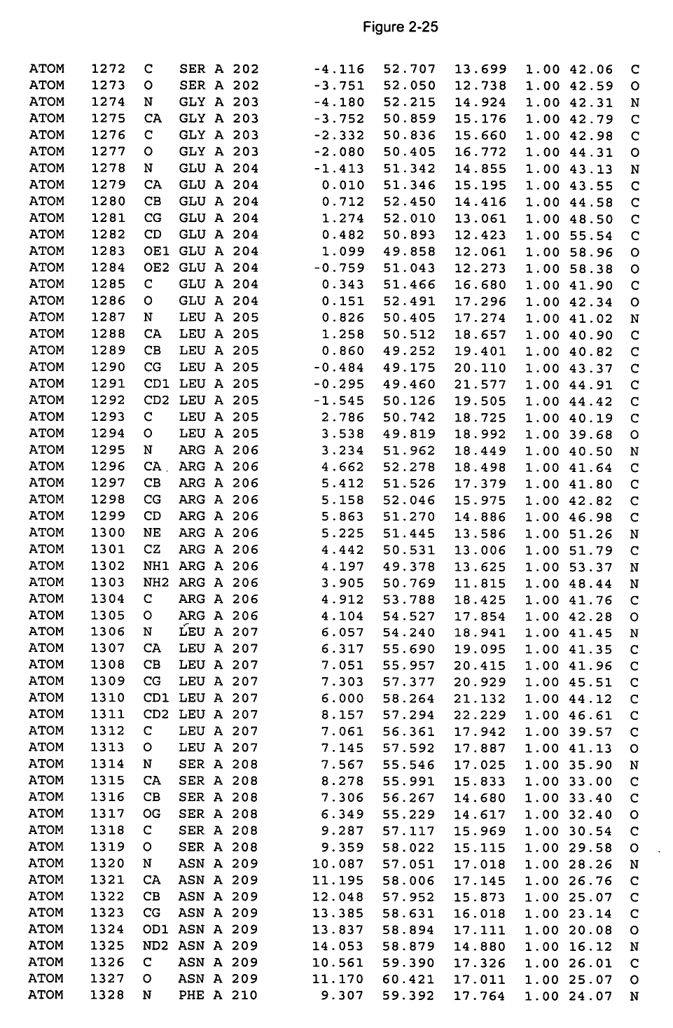

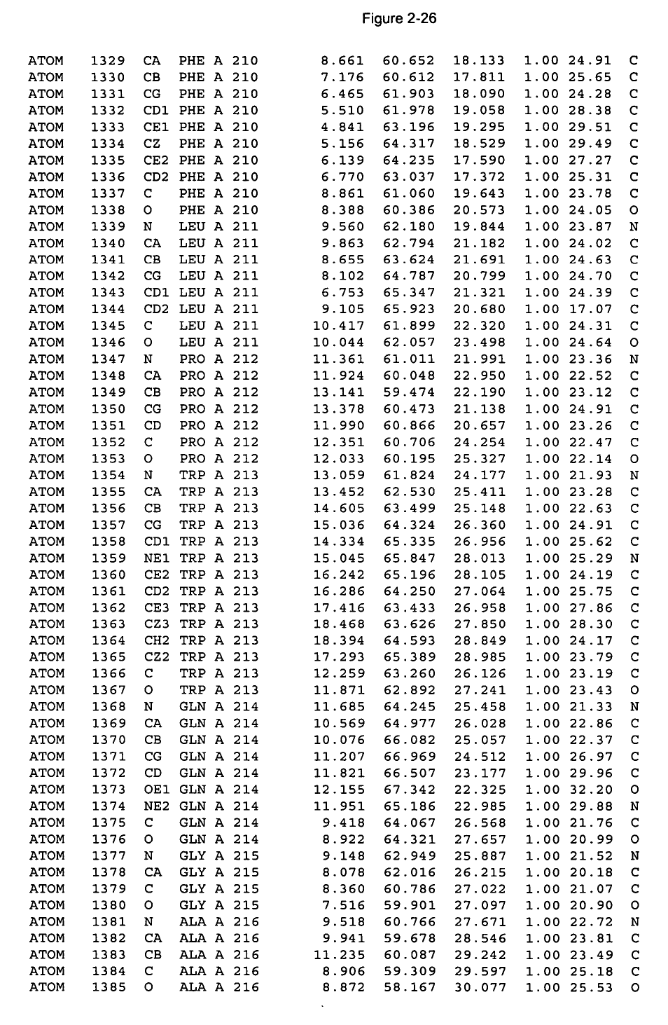

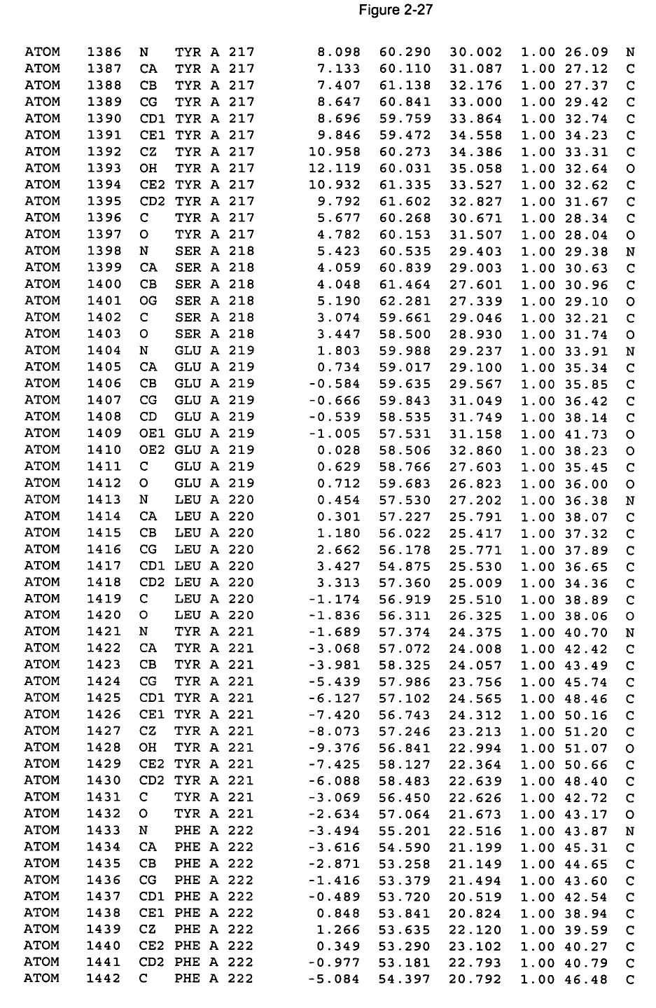

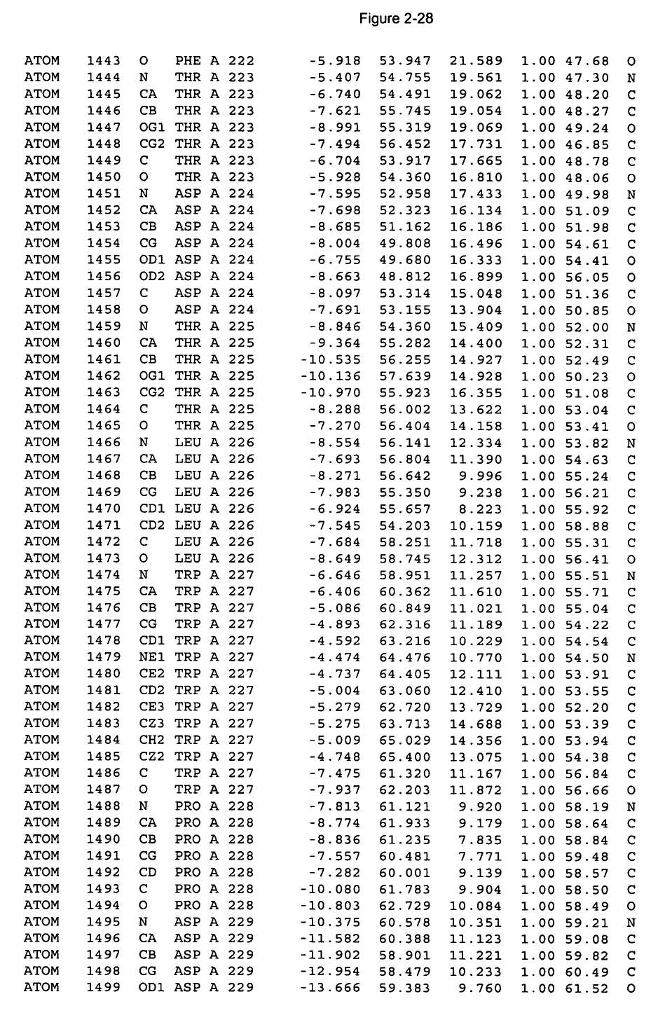

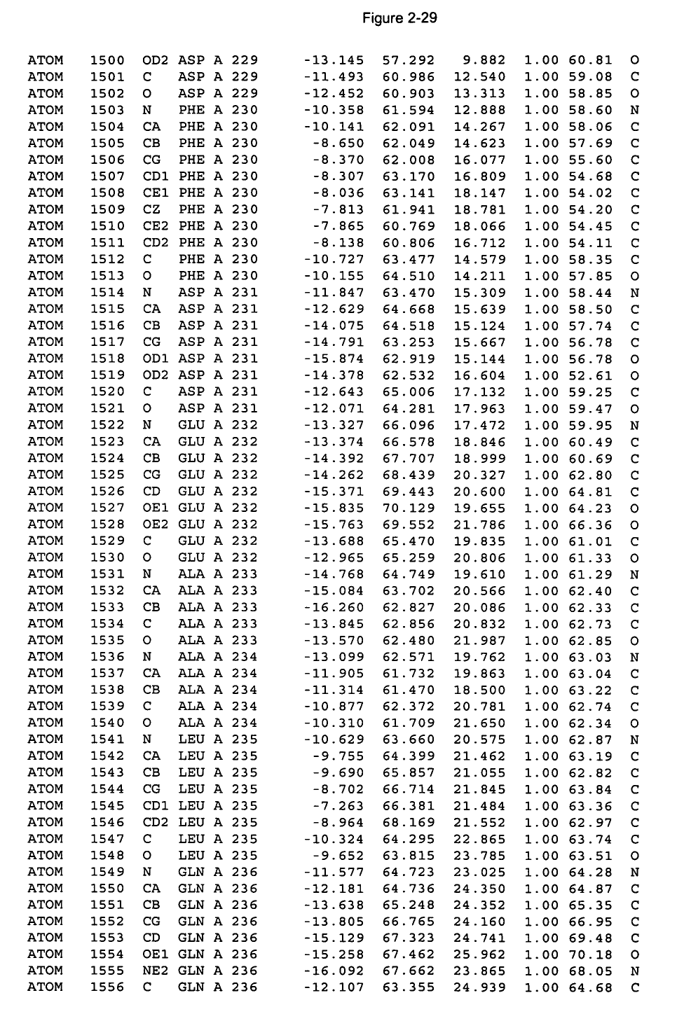

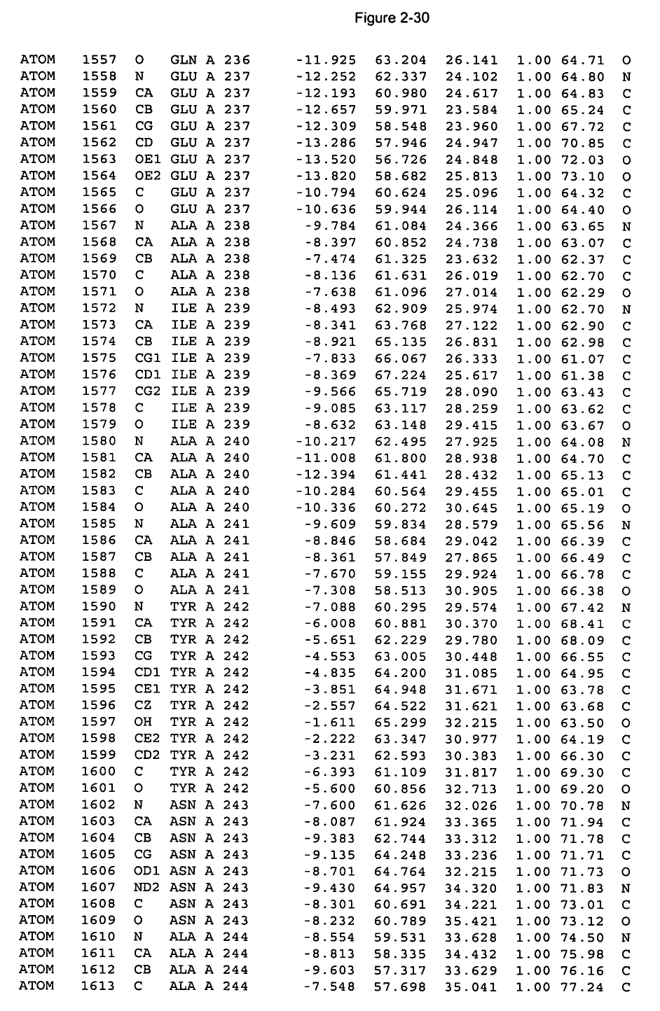

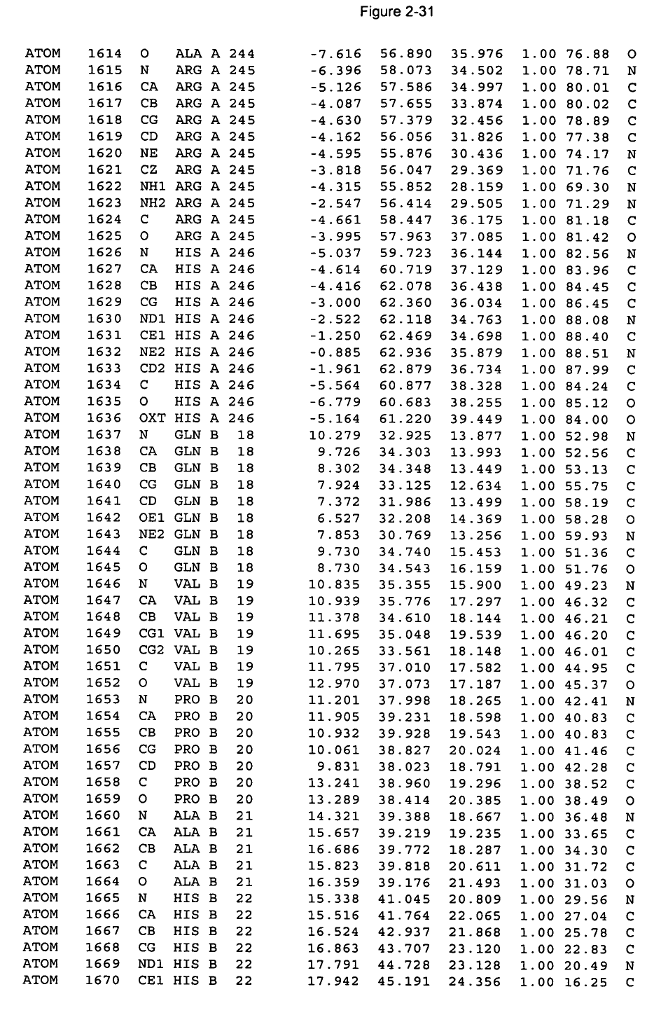

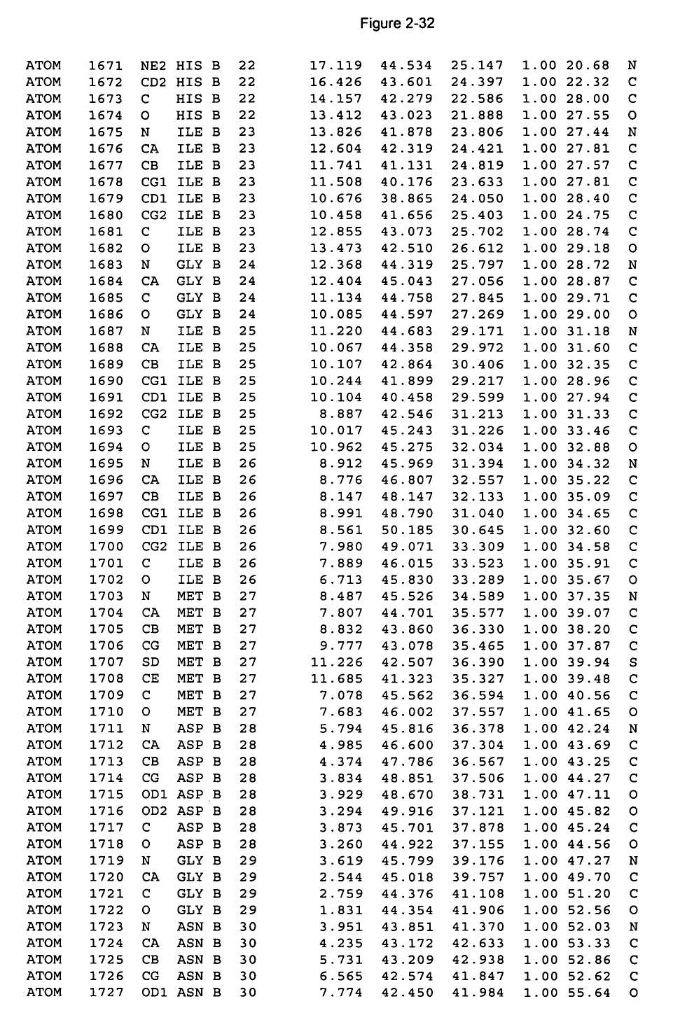

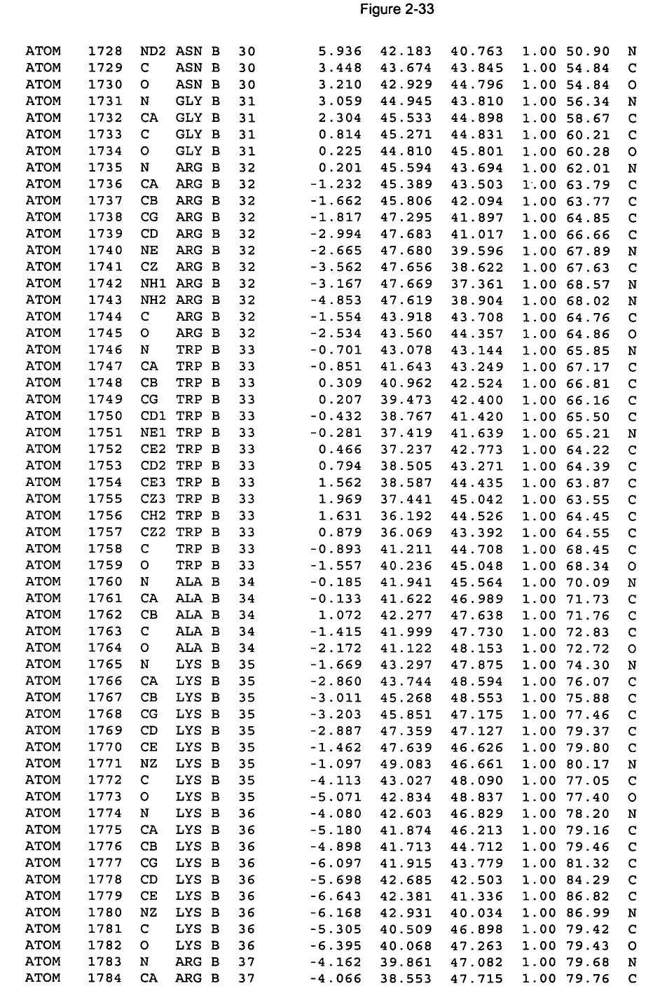

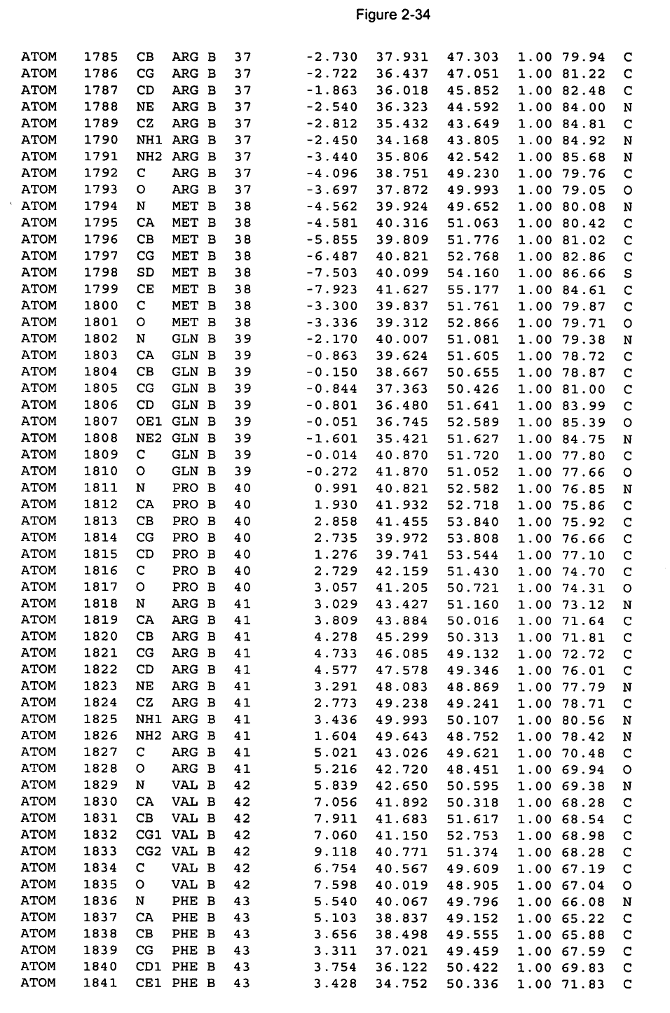

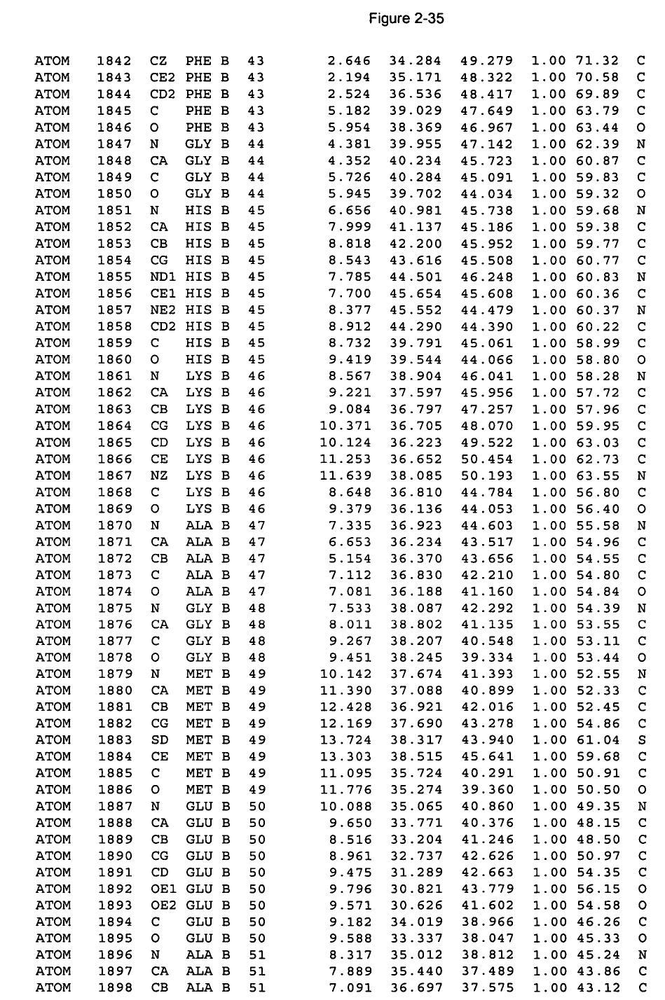

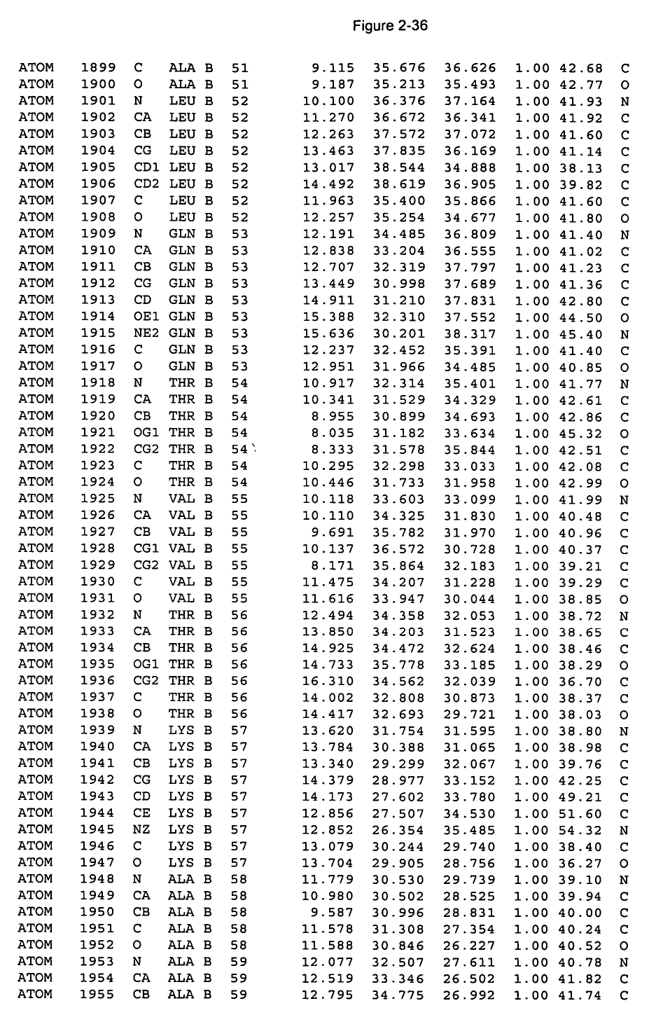

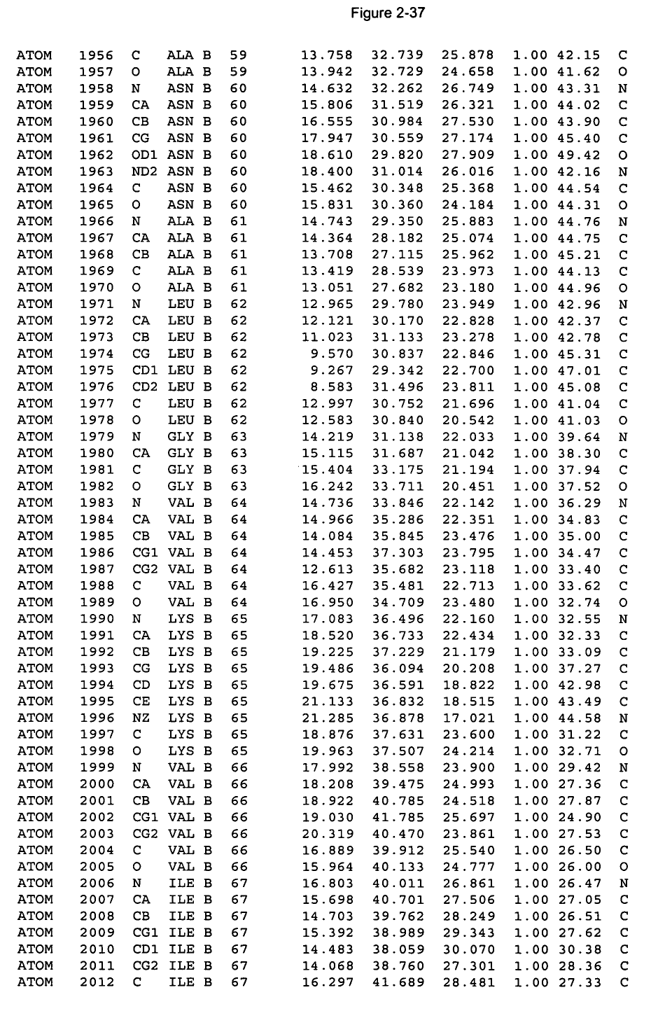

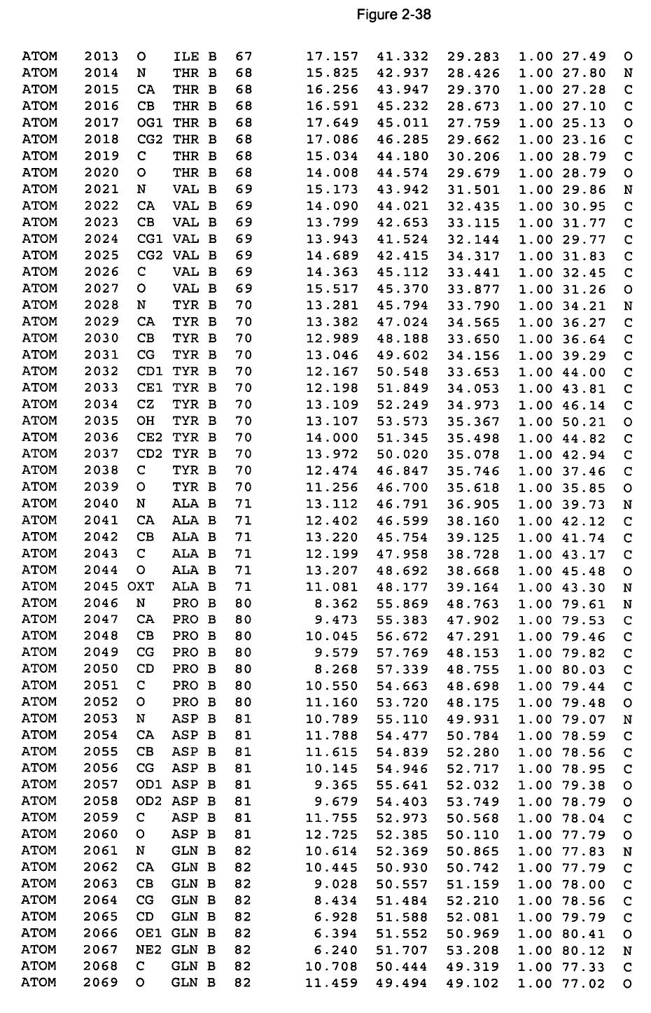

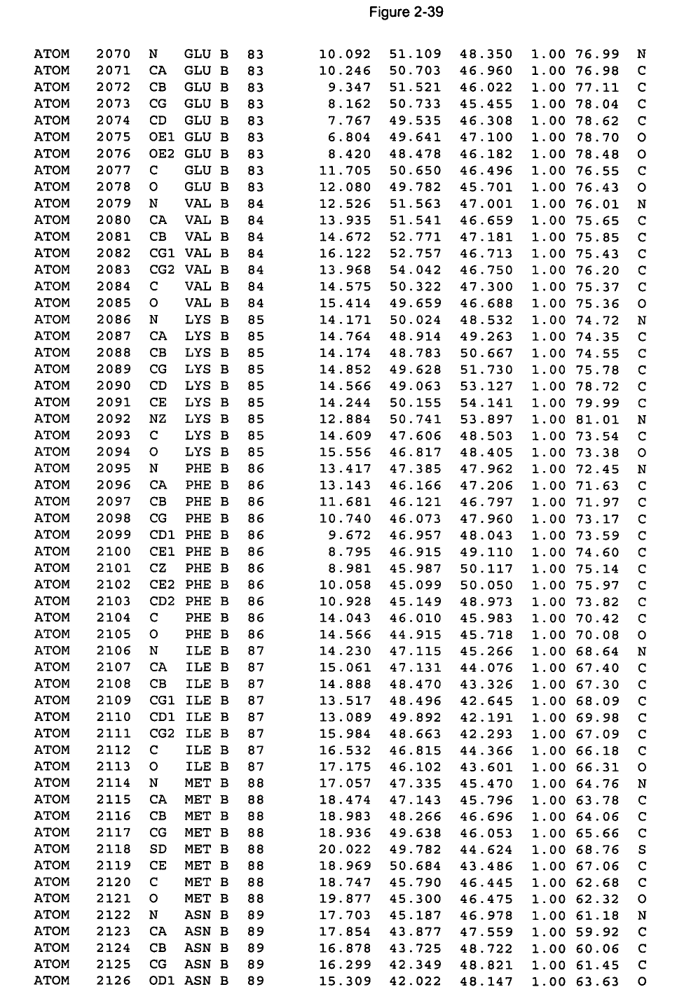

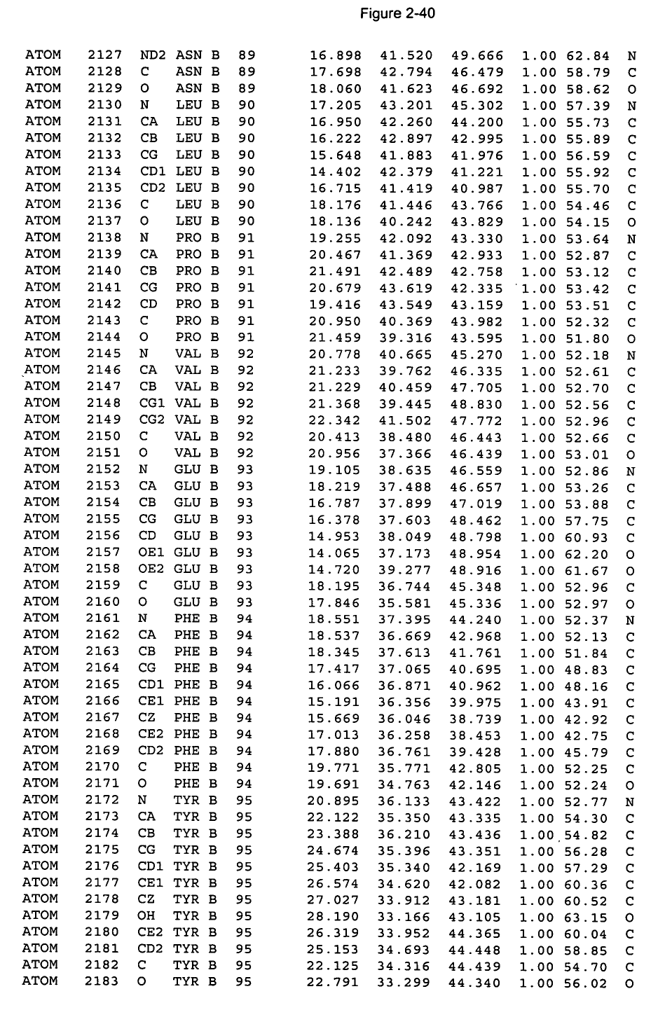

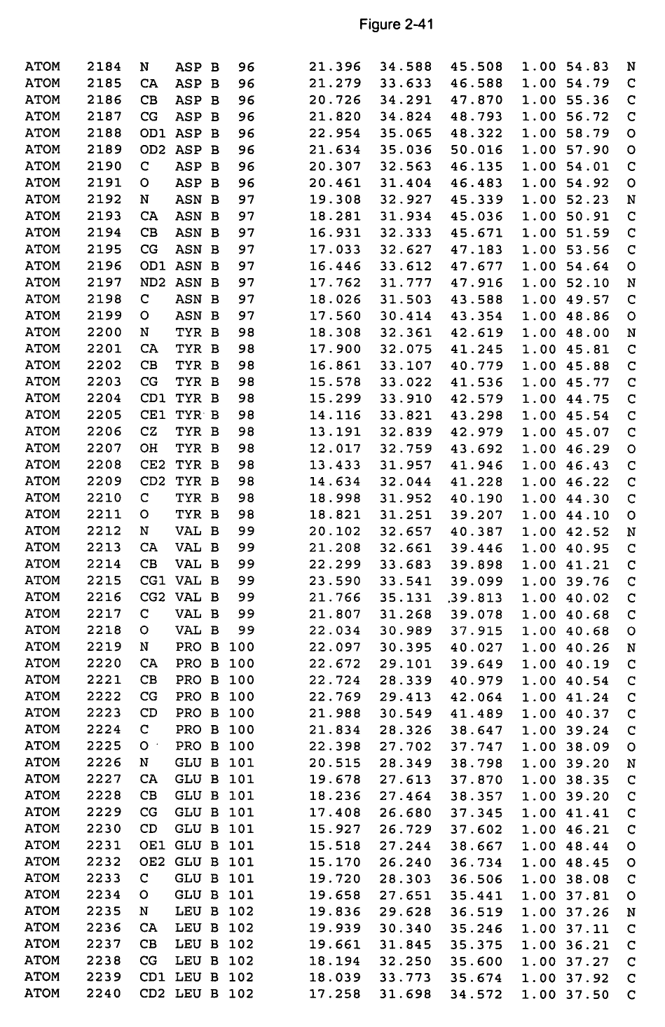

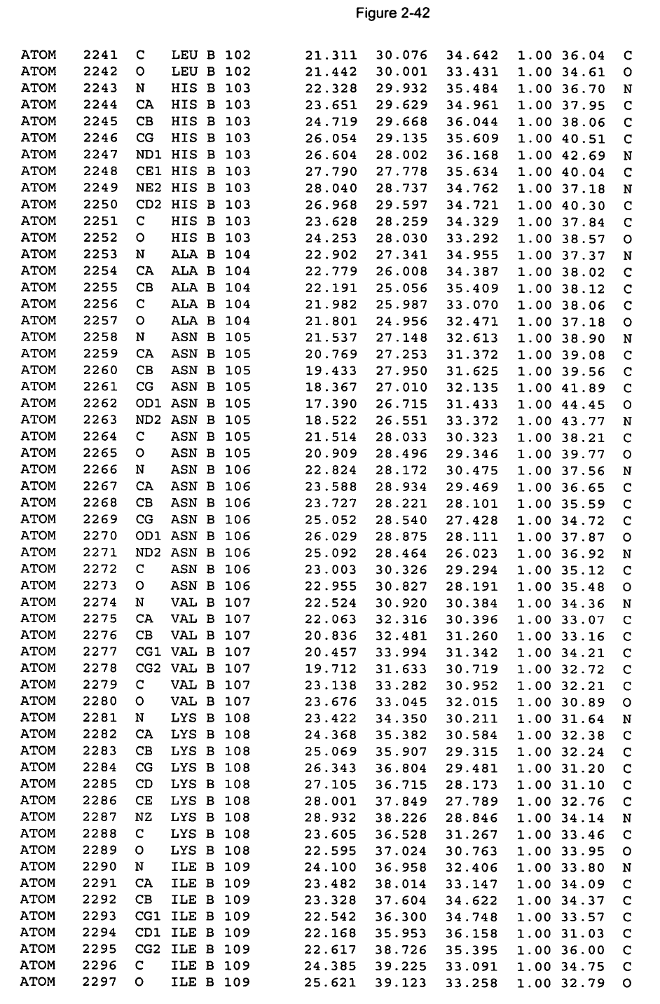

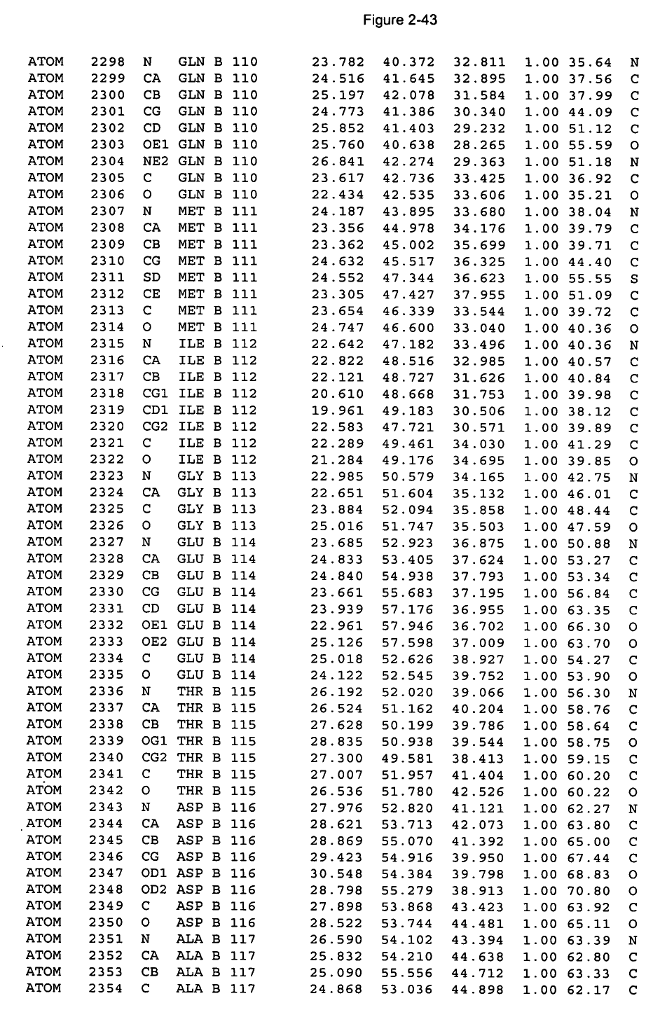

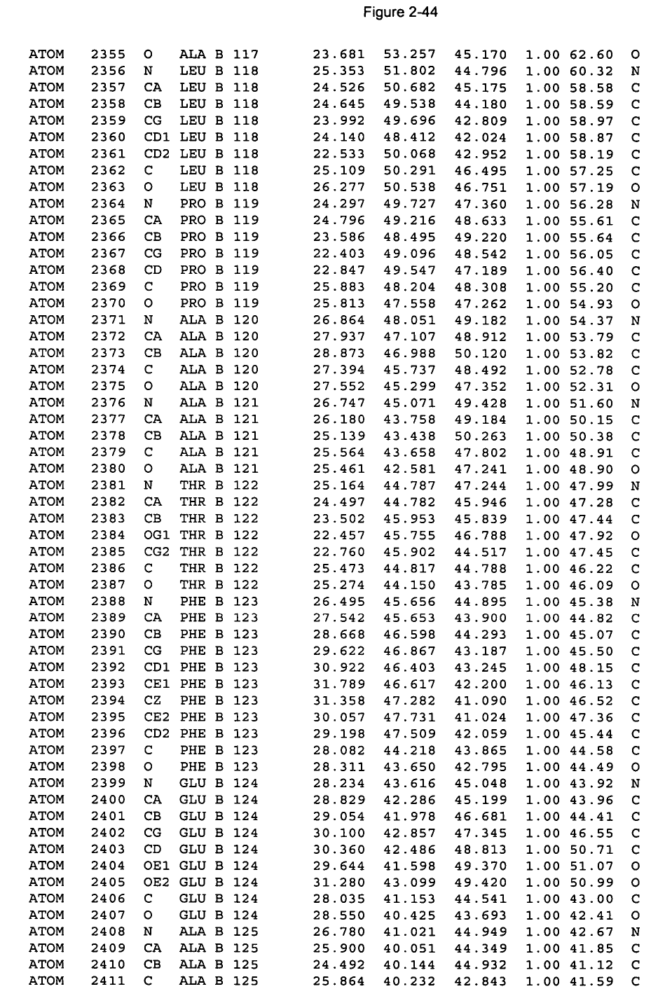

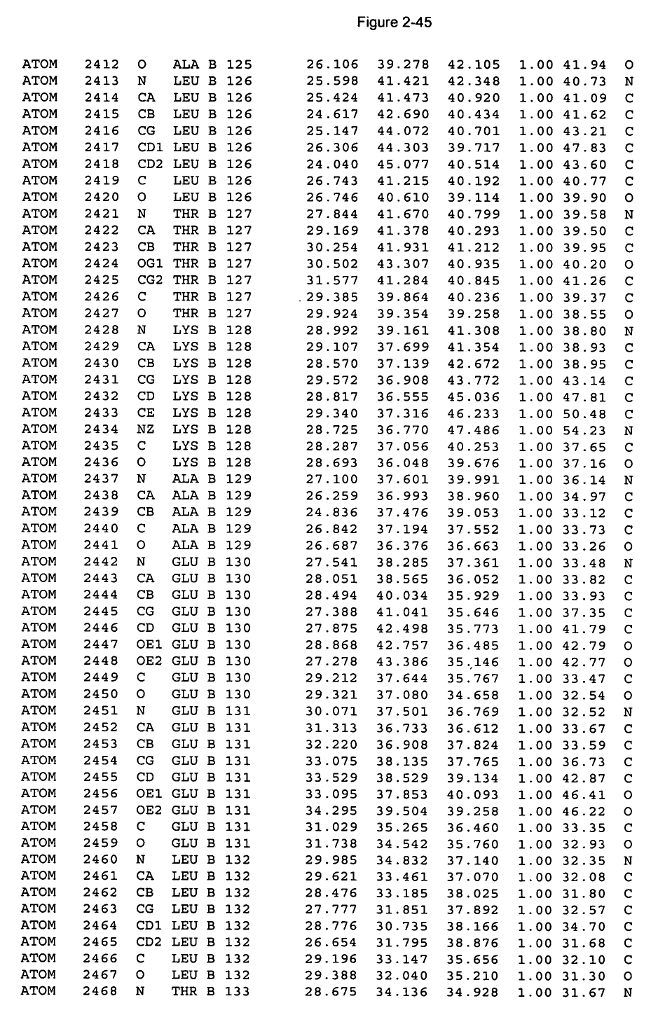

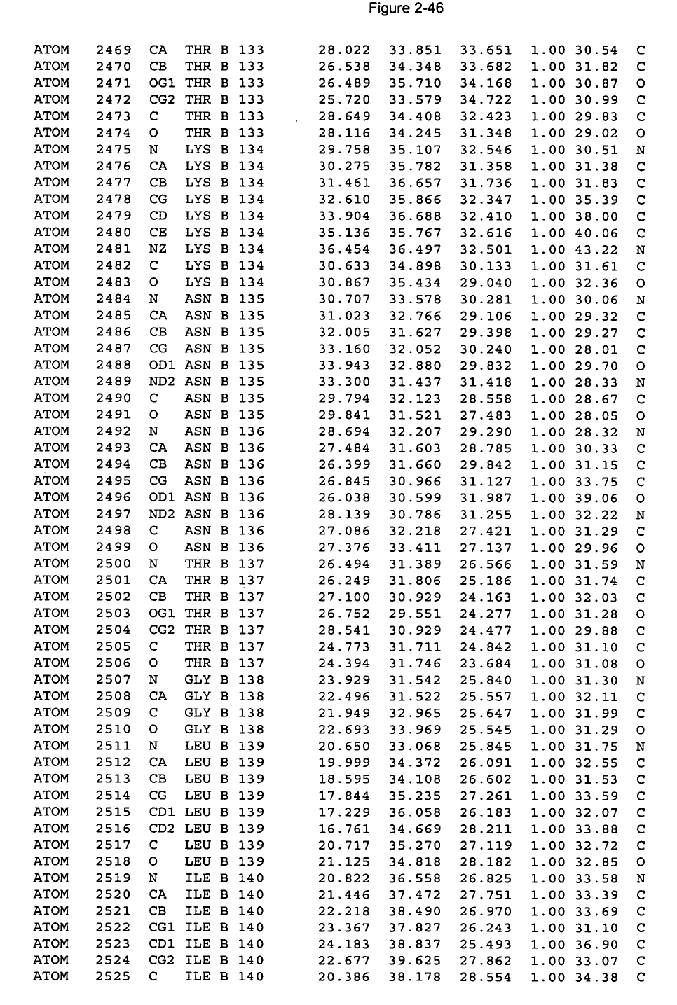

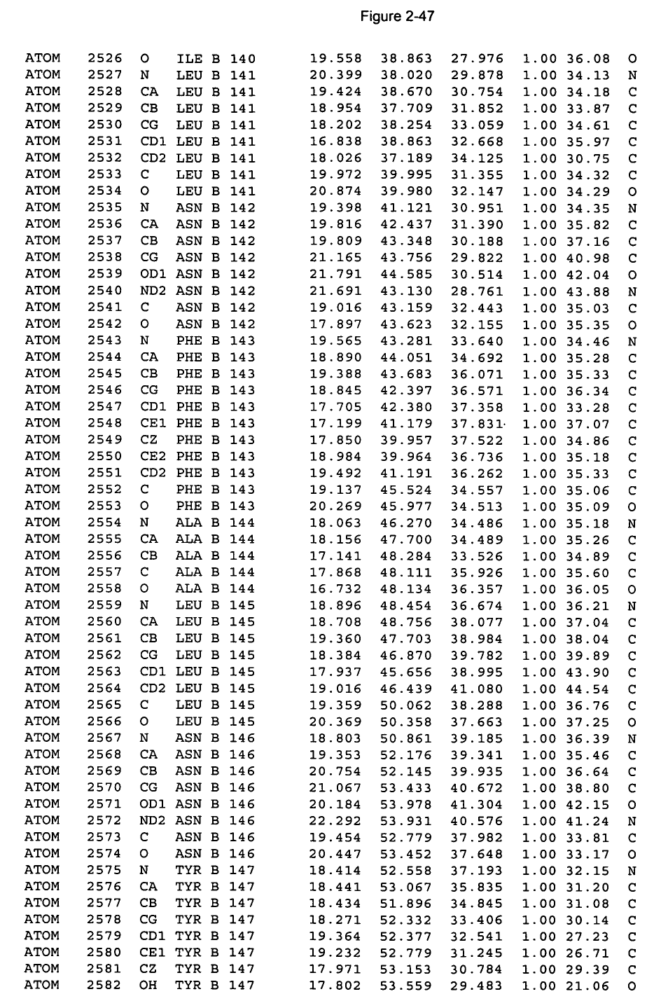

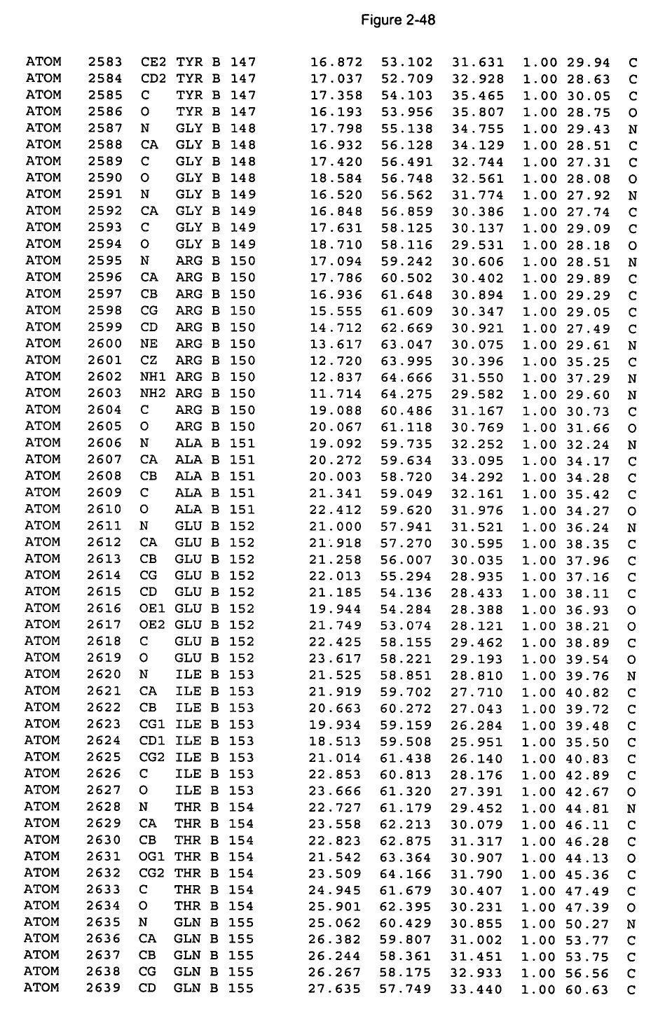

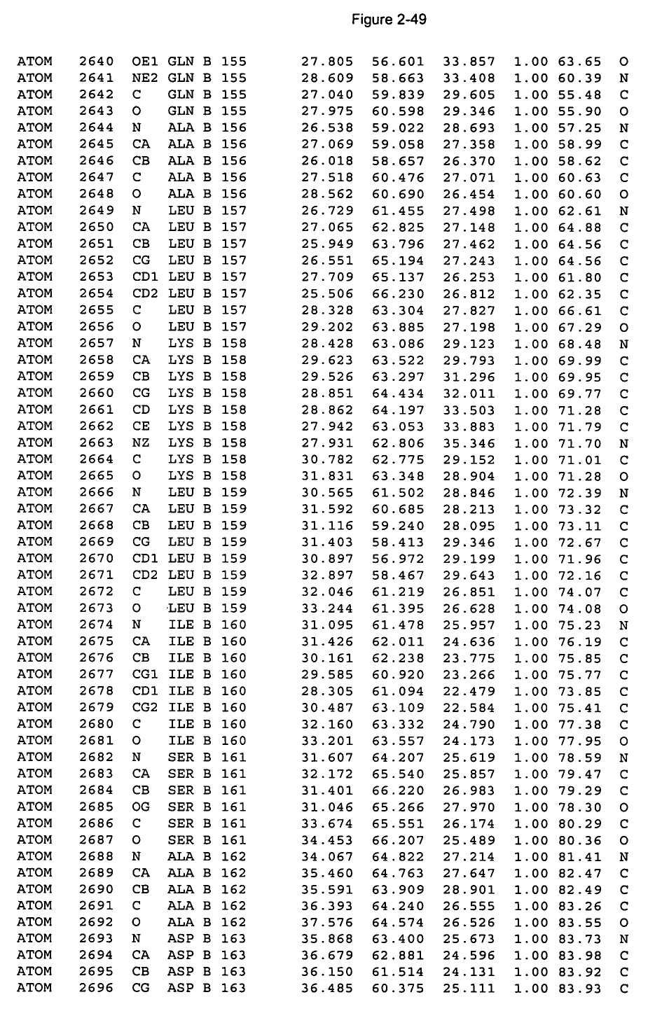

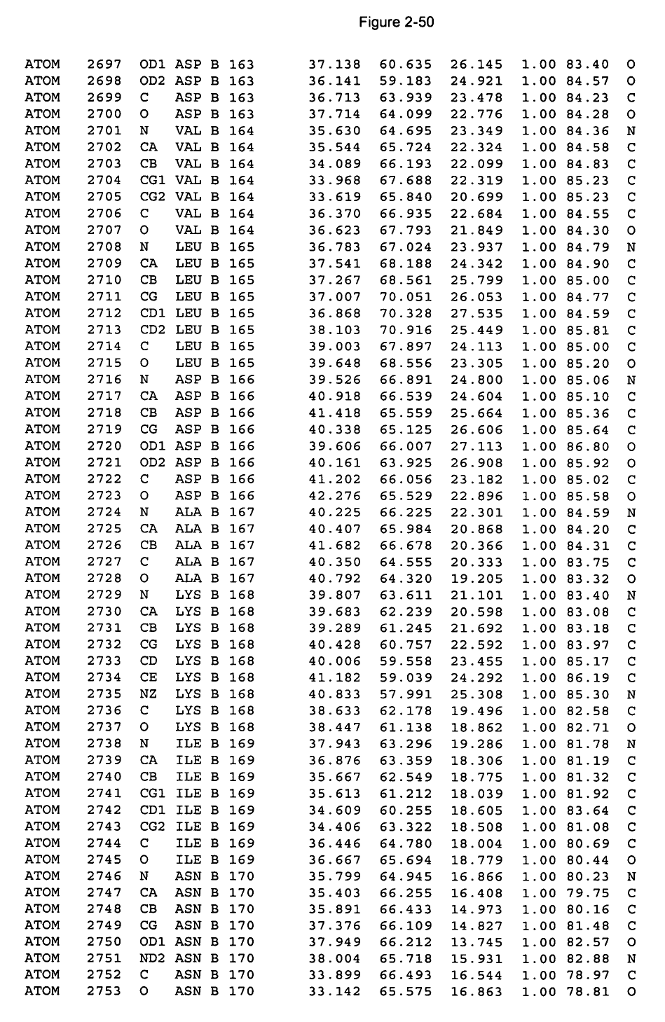

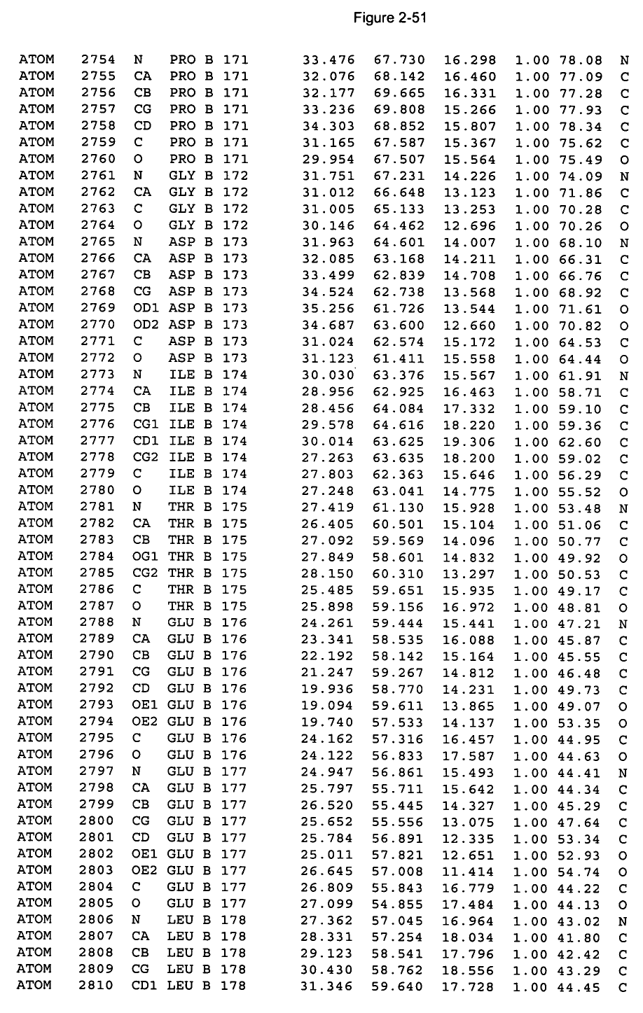

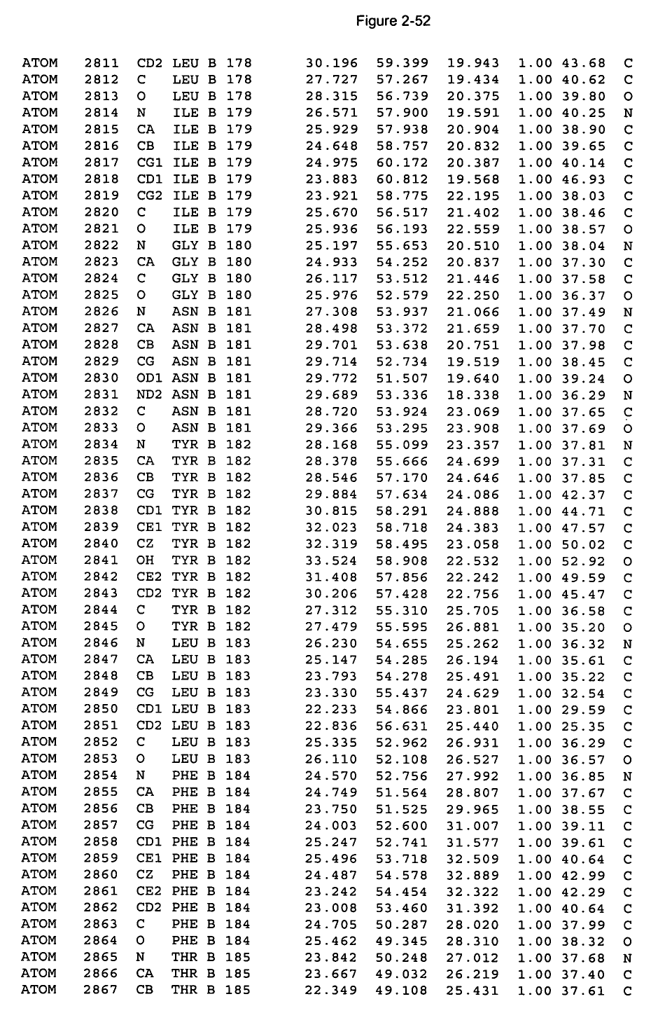

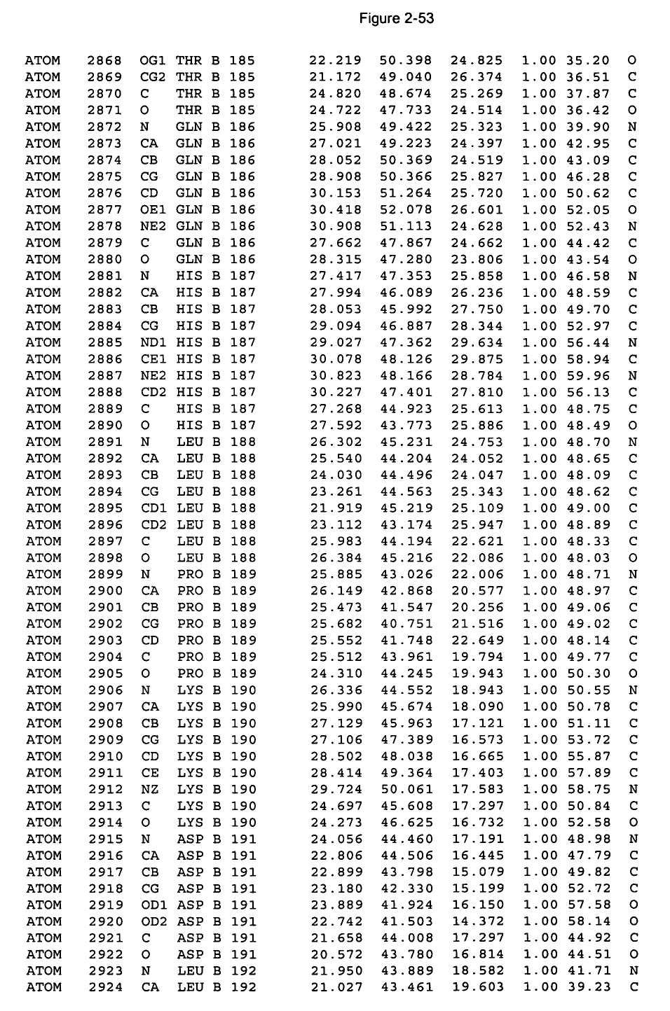

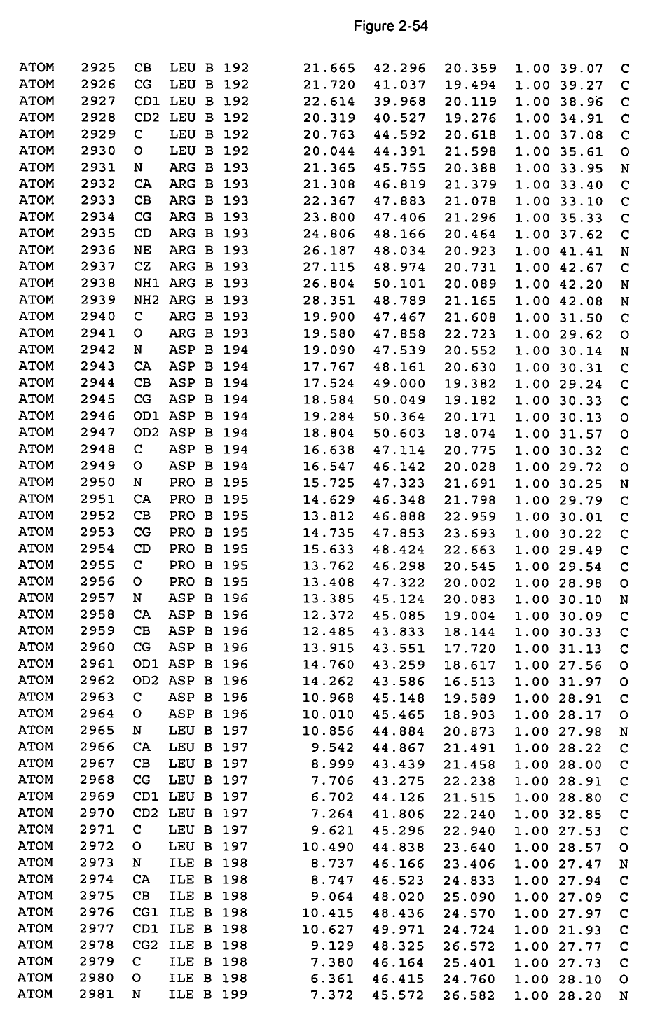

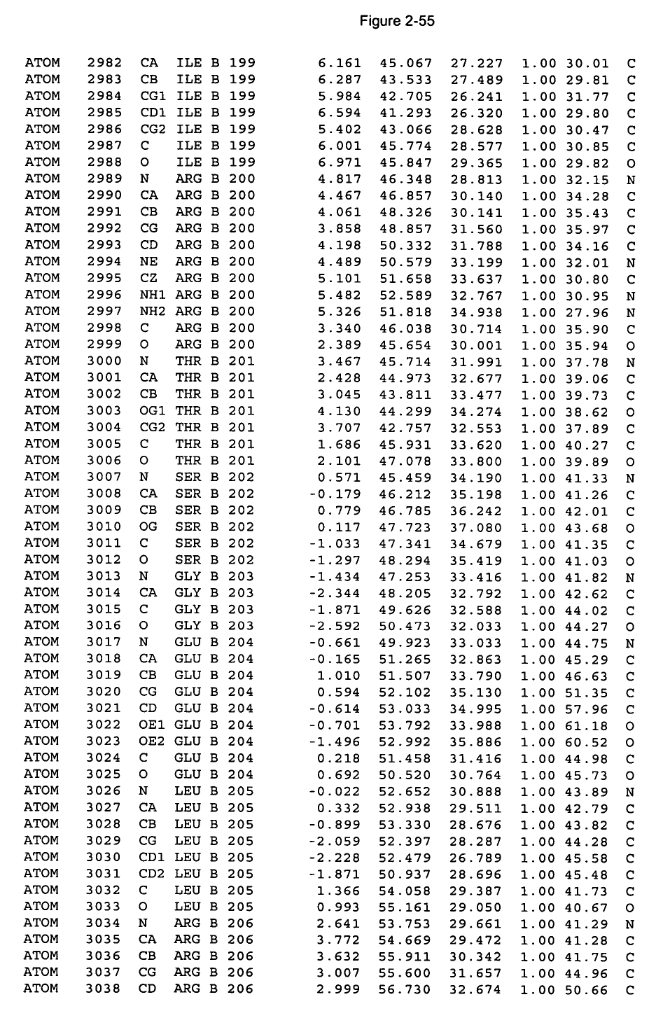

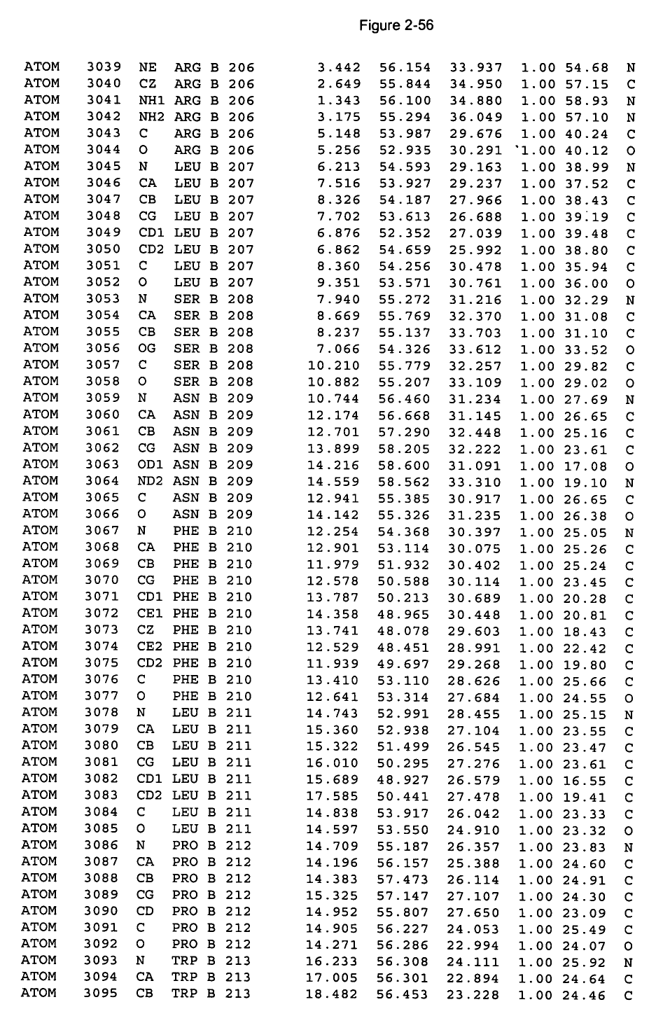

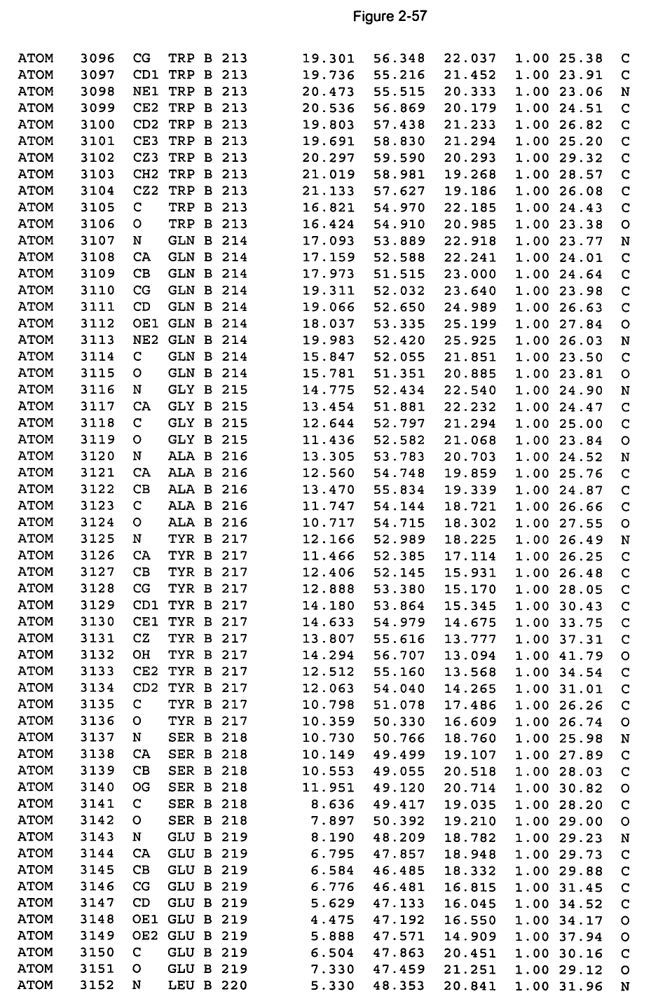

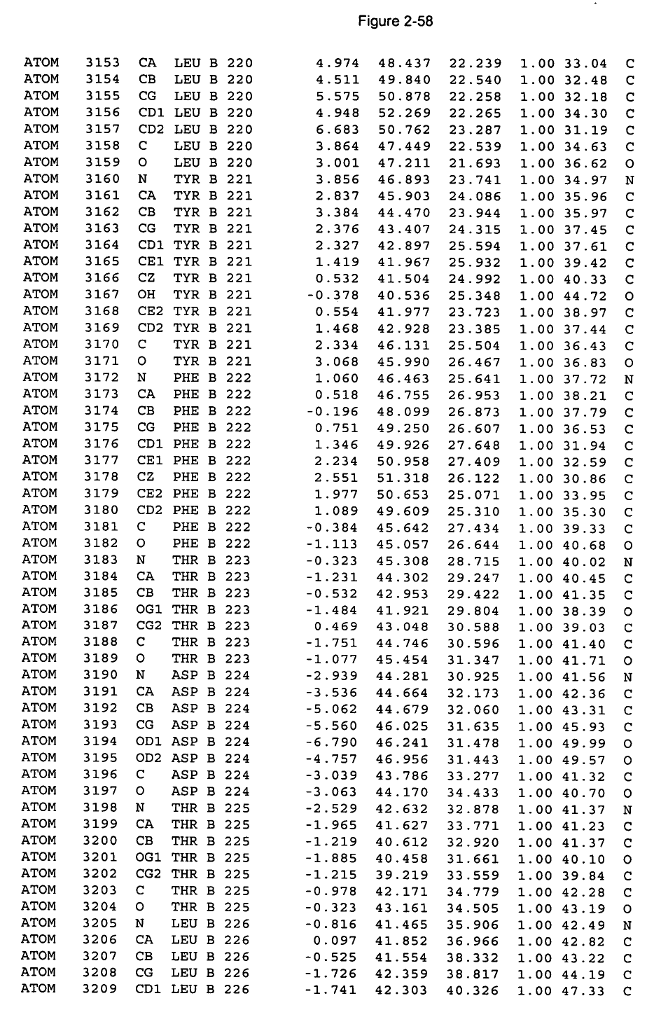

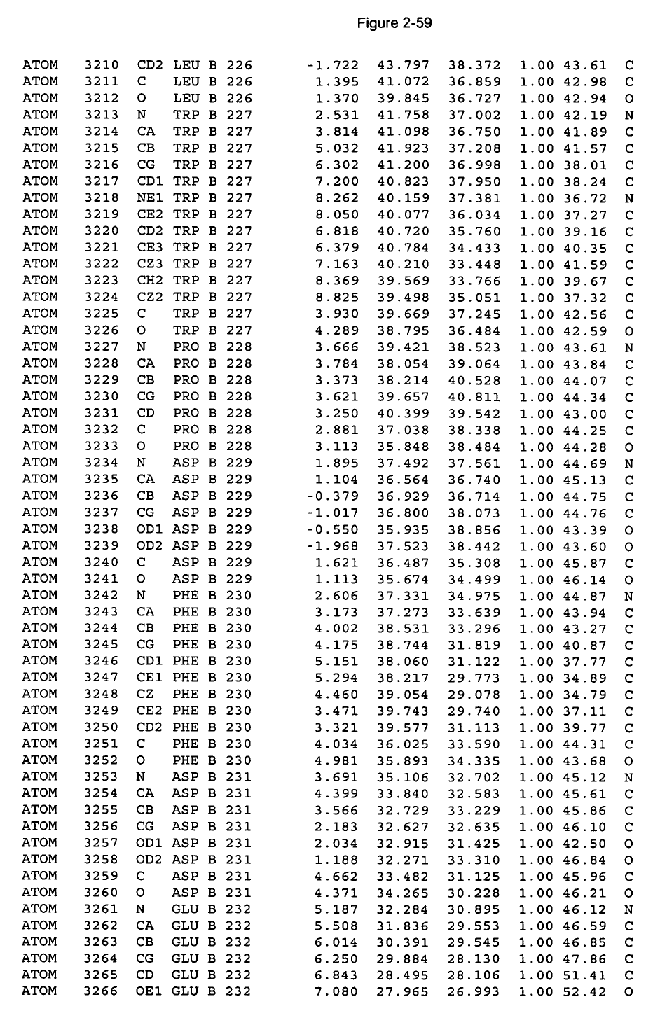

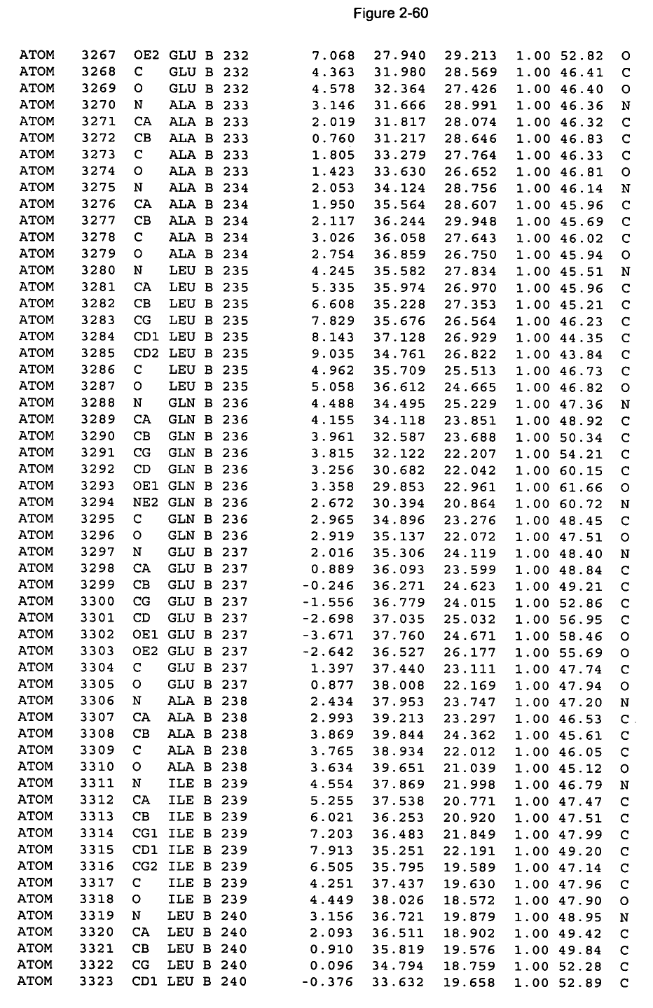

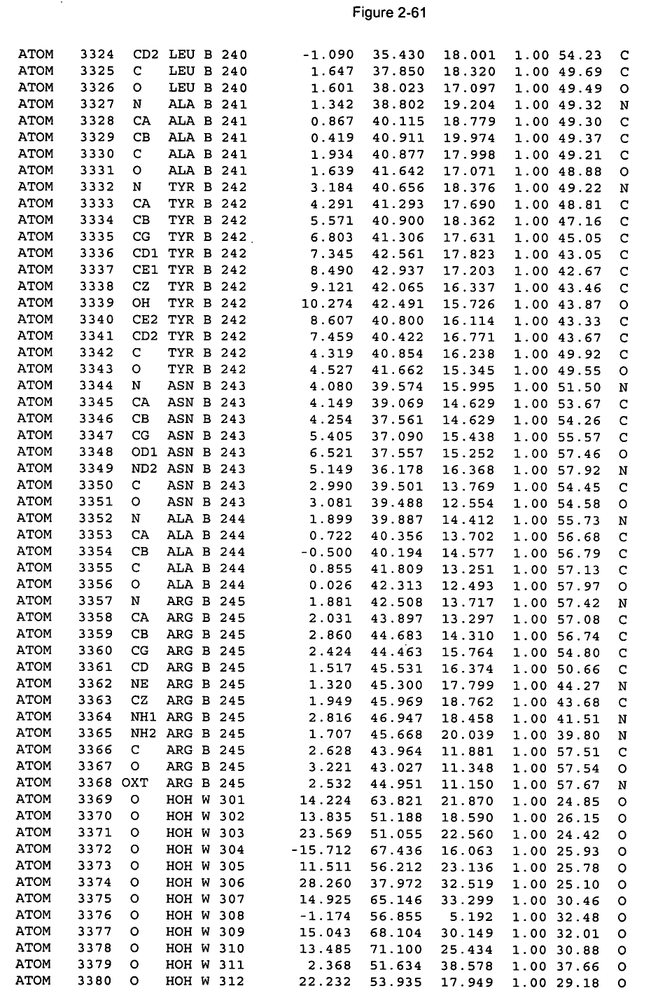

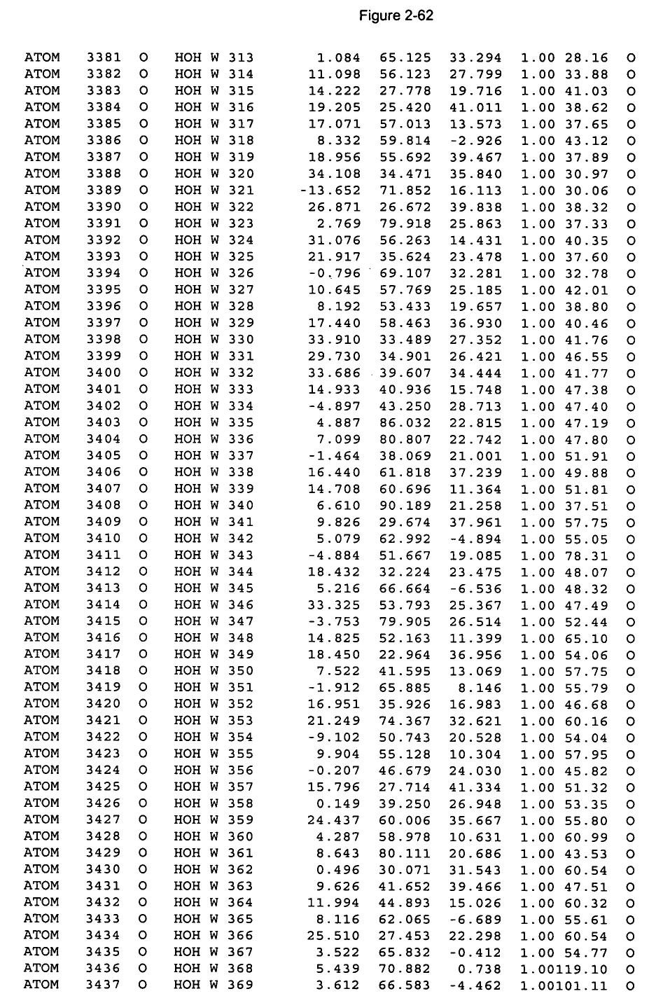

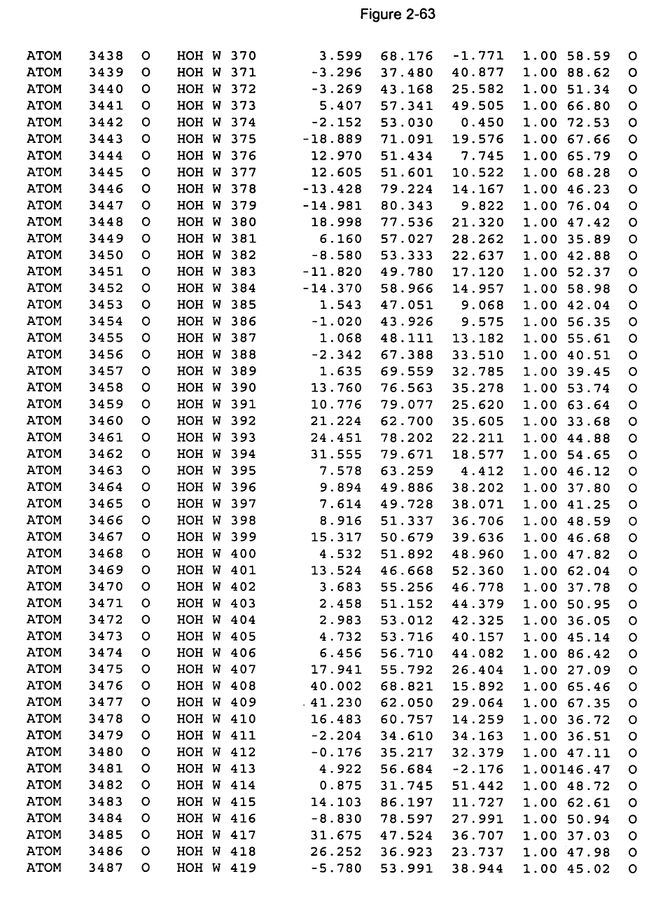

Those of skill in the art understand that a set of structure coordinates determined by X-ray crystallography is not without standard deviation. For the purpose of this invention, any set of structure coordinates for undecaprenyl pyrophosphate synthase or undecaprenyl pyrophosphate synthase homologues or undecaprenyl pyrophosphate synthase mutants that have a root mean square deviation of protein backbone atoms (N, C_{α}, C and O) of less than 0.75 Å when superimposed, using backbone atoms, on the structure coordinates listed in FIG. 2 shall be considered identical.

The term “unit cell” refers to a basic parallelepiped shaped block. The entire volume of a crystal may be constructed by regular assembly of such blocks. Each unit cell comprises a complete representation of the unit of pattern, the repetition of which builds up the crystal.

The term “space group” refers to the arrangement of symmetry elements of a crystal.

The term “molecular replacement” refers to a method that involves generating a preliminary model of an undecaprenyl pyrophosphate synthase crystal whose structure coordinates are unknown, by orienting and positioning a molecule whose structure coordinates are known (e.g., undecaprenyl pyrophosphate synthase coordinates from FIG. 2) within the unit cell of the unknown crystal so as best to account for the observed diffraction pattern of the unknown crystal. Phases can then be calculated from this model and combined with the observed amplitudes to give an approximate Fourier synthesis of the structure whose coordinates are unknown. This, in turn, can be subjected to any of the several forms of refinement to provide a final, accurate structure of the unknown crystal. See, for example, Lattman, Methods in Enzymology, 115:55-77 (1985); M. G. Rossmann, ed., “The Molecular Replacement Method”, Int. Sci. Rev. Ser., No. 13, Gordon & Breach, New York, (1972). Using the structure coordinates of undecaprenyl pyrophosphate synthase provided by this invention, molecular replacement may be used to determine the structure coordinates of a crystalline mutant or homologue of undecaprenyl pyrophosphate synthase or of a different crystal form of undecaprenyl pyrophosphate synthase.

“Atom type” in, for example, FIG. 2, refers to the element whose coordinates are measured. The first letter in the column in FIG. 2 defines the element.

“X, Y, Z” crystallographically define the atomic position of the element measured.

“B” is a thermal factor that measures movement of the atom around its atomic center.

Atomic coordinates for undecaprenyl pyrophosphate synthase according to FIG. 2 may be modified from this original set by mathematical manipulation. Such manipulations include, but are not limited to, crystallographic permutations of the raw structure coordinates, fractionalization of the raw structure coordinates, integer additions or subtractions to sets of the raw structure coordinates, and any combination of the above. The atomic coordinates of FIG. 2 correspond to the undecaprenyl pyrophosphate synthase polypeptide chains, and to several molecules bound thereto, including magnesium ion, FPP, sulfate, and a plurality of water molecules.

Materials and Methods

Cloning and Expression

Amino acids 10-252 of Streptococcus pneumoniae UppS (see SEQ ID NO:1) were subcloned into a pET28a (Novagen) derivative. The modified fusion protein has a N-terminal tag (MHHHHHHSSGLVPRGSMA) (SEQ ID NO: 22) consisting of a His-tag and a thrombin protease site. Expression was carried out in BL21Gold cells (Stratagene). The cells were induced overnight with 50 μM isopropyl-beta-D-thiogalactoside at 25° C. in LB medium supplemented with 0.2% glucose.

Purification of Native S. pneumoniae UPS

About 30 grams of cells from 5 liter shake flask culture of E. coli expressing the (his)₆-UPS fusion protein were lysed in 100 mls buffer (50 mM Tris-HCl, 0.3 M NaCl, 4 mM β-ME, pH 8.0, 1 μg/mL pepstatin, 1 μg/mL leupeptin, 1 mM PMSF, 0.2 mg/ml lysozyme, 10 mM MgCl₂, 3 μg DNAs/ml) after sitting on ice for 30 min. using a Branson Sonifier 450. Lysate was clarified by centrifugation at 40,000 x g. Soluble protein was applied to a 15 mL immobilized metal affinity column, Ni-NTA, which had been equilibrated in buffer A (50 mM Tris-HCl, 0.3 M NaCl, 4 mM □-ME, 20 mM imidazole, pH 8.0 and 1 μg/mL pepstatin and leupeptin). After washing with buffer A+40 mM imidazole, bound protein was step eluted with buffer B (buffer A+0.25 M imidazole, pH 8.0). Fractions were analyzed by SDS-PAGE on 10% bis-tris gels in MES buffer using the NuPAGE system. Fractions containing his-UPS were pooled and dialyzed against buffer containing 50 mM Tris-HCl, 0.3 M NaCl, 4 mM β-ME, pH 8.0 for further purification.

The (his)₆ tag was removed by digestion with thrombin at a specific thrombin recognition site. (His)₆-UPS was treated with thrombin at 2 activity units/mg: (his)₆-UPS. The reaction was stopped by addition of PMSF to 1 mM after 3 hrs. at room temperature. Chromatography was performed as described above for isolation of (his)₆-UPS. Untagged UPS was isolated in the flowthrough and wash fractions. The remaining (his)₆-UPS and the cleaved (his)₆ peptide were removed in the bound fraction. Fractions were analyzed by SDS-PAGE on 10% bis-tris gels in MES buffer using the NuPAGE system. Fractions containing des-his-UPS were pooled for further purification.

Des-his-UPS was further purified by size exclusion chromatography. The protein was concentrated to 10-14 mg/mL and loaded onto a 125 mL Superdex 200 prep grade column which had been equilibrated in buffer containing 50 mM Tris-HCl, 0.3 M NaCl, 8 mM DTT, pH 7.5. UPS eluted as a 55 kDa dimer. Fractions were analyzed by SDS-PAGE on 10% bis-tris gels in MES buffer using the NuPAGE system. The final pool was characterized by dynamic light scattering (DLS), and LC/MS. DLS revealed that the protein was monodisperse. Mass analysis revealed a small level of truncated N-terminus consistent with the loss of 7 amino acids. This was confirmed by N-terminal sequence analysis. Protein isolated in the manner described above was used for crystallization.

Crystallization of Native S. pneumoniae UPS

The des-his-UPS was made in 50 mM Tris-HCl, 0.3 M NaCl, 8 mM DTT, pH 7.5. Sparse matrix screening using the hanging drop method was performed. Drops were set at a protein concentration of 3 mg/mL over the reservoir. Plates were incubated at 22° C. Few leads were identified. One optimized reservoir condition (0.1M HEPES, pH 7.5, 8% ethylene glycol, 10% PEG 8000) resulted in large tabular crystals ˜350×250×25 microns.

The sequence of the construct used in crystallization experiments is given in SEQ ID NO:1.

Data Collection

X-ray diffraction data were collected from flash-frozen crystals at 100° K. Crystals were briefly soaked in a cryoprotectant solution which consisted of 25% ethylene glycol added to the crystallization reservoir solution. They were then introduced into a 100° K. cold nitrogen stream.

Crystals are orthorhombic, belong to the space group I2₁2₁2₁, with unit cell dimensions of a=59.99 Å, b=118.20 Å, c=178.93 Å, α=β=γ=90°.

Three-wavelength diffraction data to a maximum resolution of 2.3 Å were collected at beamline 5.0.1 of Advance Light Source of Lawrence Berkeley Laboratory, using a ADSC ccd detector. Data were reduced using the HKL suite of programs. There are a total of 212419 reflections collected, 28867 are unique reflections, giving a data redundancy of 7.4 and a completion of 99.9%. The crystal mosaicity is 0.4 degree, the Rmerge is 7.6%. All indicate a good quality for the data.

Structure Determination

The synthase structure was solved by molecular replacement methods using the CCP4 programs and the published E. coli Structure (PDB 1JP3) as the search model.

After rigid-body refinement, the starting Rfactor is 46.9% to 3.2 Å resolution. Automated map-tracing, as implemented in ARP/wARP was used to partially trace the map, and the program was able to improve the quality of the map to a degree that manual fitting can be readily carried out using the program XtalView. All model refinement was carried out using the program REFMAC as implemented in the CCP4 suite of programs.

There are two undecaprenyl pyrophosphate synthase molecules per asymmetric unit. The current model consists of residues 19-246 for one molecule and 18-245 for the other, and 119 bound water molecules. This model has an overall R factor of 0.286, with a free R-factor of 0.336, for all data to 2.3 Å. Refinement statistics are shown in Table 1A.

TABLE 1A

| | |
|---|---|
| Resolution | 50.0-2.3 Å |
| Reflections | 26,588 (all data) |
| R_{work} | 28.6% |
| R_{free} | 33.6% |
| RMSD in bond lengths | 0.013 Å |
| RMSD in bond angles | 1.7° |
| Total atoms (non-hydrogen) | 3487 |
| Ramachandran Plot | 86.1 |
| Most favoured | |
| Ramachandran Plot | 12.8 |
| Allowed | |

Final refined coordinates for S. Pneumoniae UPS are shown in FIG. 2.

The Structure of Streptococcus Undecaprenyl Pyrophosphate Synthase

The overall topology of the synthase is similar to known undecaprenyl pyrophosphate synthases, consisting of a six-stranded parallel β-sheet, surrounded by eight α-helices.

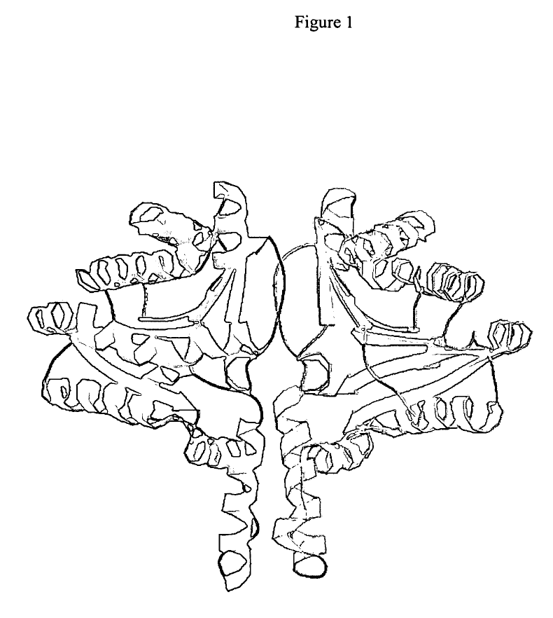

Turning to FIG. 1, the crystal structure reveals that undecaprenyl pyrophosphate synthase exists as a dimer, with two identical subunits related by a 2-fold axis of symmetry. FIG. 1 shows a ribbon drawings of the undecaprenyl pyrophosphate synthase dimer. As depicted in FIG. 1, the two subunits are intimately associated.

The residues from Phe⁷² to Leu⁹⁰ are disordered in one of the molecules, while the residues from Phe⁷² to Arg⁷⁹ are disordered in the other. Structural analysis of the enzyme indicates that the active site is defined as consisting of at least one of the following residues: Asp²⁸, Gly²⁹, Gly³¹, Arg³², Arg⁴¹, Arg⁷⁹, Leu⁹⁰, Pro⁹¹, Phe¹⁴³, Arg ²⁰⁰, Arg²⁰⁶, and Ser²⁰⁸ from the one chain, and Glu²¹⁹ and Gly²⁵⁰ from the other chain, denoted chain B. In mutants or homologs of S. pneumoniae undecaprenyl pyrophosphate synthase the numbering of amino acid residues can be normalized to the S. pneumoniae reference sequence.

The S. pneumoniae undecaprenyl pyrophosphate synthase has several structural features indicated in Table 1B below.

TABLE 1B
Undecaprenyl Pyrophosphate Synthase Secondary Structure Assignments

| | |
|---|---|
| His²²-Met²⁷ | S1 |
| Asn³⁰-Lys³⁵ | H1 |
| Arg⁴¹-Leu⁶² | H2 |
| Val⁶⁶-Ala⁷¹ | S2 |
| Asn⁷⁶-Thr⁷⁸ | H3a |
| Asp⁸¹-Ala¹⁰⁴ | H3b |
| Lys¹⁰⁸-Ile¹¹² | S3 |
| Lys¹²⁰-Thr¹³³ | H4 |
| Ile¹⁴⁰-Leu¹⁴⁷ | S4 |
| Gly¹⁴⁹-Leu¹⁶⁵ | H5 |
| Glu¹⁷⁶-Gly¹⁸⁰ | H6 |
| Phe¹⁸⁴-His¹⁸⁷ | H7 |
| Leu¹⁹⁷-Arg²⁰⁰ | S5 |
| Glu²¹⁹-Phe²²² | S6 |
| Trp²²⁷-Asp²²⁹ | H9 |
| Glu²³²-Asn²⁴³ | H10 |

In the Table 1B, beta-strands are labeled S1-S6 and helices are labeled H1-H10. The helices H3a and H9 are 3₁₀ helices; the others are alpha helices. Secondary structures have been calculated according to the method of Kabsch and Sander, as implemented in the program Procheck. Other algorithms used to calculate secondary structure can produce slightly different assignments.

The S. pneumoniae undecaprenyl pyrophosphate synthase has several notable structural features, including the following. The amino acid residues from position 22-27 are part of a beta sheet strand termed S1. H1 is an alpha helix immediately adjacent to a catalytic aspartic acid in position 28 and also has several residues capable of binding FPP including Gly²⁹, Asn³⁰, Gly31 and Arg³². The amino acid residues from position 41 to 62 form an alpha helix termed H2. The amino acid residues from position 66 to 71 are part of a beta sheet strand termed S2. The amino acid residues from position 73 to 84, described above as capable of being a flexible loop, are disordered in the structure but should include Arg⁷⁹ that is capable of making two hydrogen bonds with a phosphate group of FPP.

Moreover, the synthase has other notable features. The amino acids from Asp⁸¹ to Ala¹⁰⁴ should form an alpha helix termed H3b. Of interest, the H3b helix includes a proline that may allow flexibility in the structure. The amino acid residues from Lys¹⁰⁸ to Ile¹¹² form a part of a beta sheet termed S3. The amino acids from Lys¹²⁰ to Thr¹³³ form an alpha helix termed H4. The amino acids from Ile¹⁴⁰ to Leu¹⁴⁷ form part of a beta sheet termed S4. The amino acid residues from Gly¹⁴⁹ to Leu¹⁶⁵ form an alpha helix termed H5. The amino acid residues from Glu176 to Gly¹⁸⁰ form a helix termed H6. The amino acid residues from Phe¹⁸⁴ to His¹⁸⁷ form an alpha helix termed H7. The amino acid residues from Leu¹⁹⁷ to Ang²⁰⁰ form a strand of beta sheet termed S5. The amino acid residues from Glu²¹⁹ to Phe²²² form a strand of beta sheet termed S6. The amino acid residues from Trp²²⁷ to Asp²²⁹ form a 3₁₀ helix termed H9. The amino acid residues from Glu²³² to Asn²⁴³ form an alpha helix termed H10.

The residues of undecaprenyl pyrophosphate synthase should interact with farnesyl pyrophosphate and the magnesium ion cofactor. The Asp²⁸ has a carboxylic acid functional group in the beta position, the oxygen atom of which should interact with the magnesium ion at a distance of about 2 Å. This metal coordination would serve to lock the magnesium ion into a position to interact with two oxygen atoms of the pyrophosphate group of farnesyl pyrophosphate at intermolecular distances of about 2 Å.

The synthase interaction with FPP is also expected to be mediated by other amino acid residues. Arg³² has nitrogen atoms that could interact with the oxygen atoms of the phosphates in farnesyl pyrophosphate, with nitrogen to oxygen hydrogen bond interactions expected to have distances of about 2.4 to 3.2 Å. The Gly³⁶ has an alpha amino group that could form a hydrogen bond with the bridge oxygen of farnesyl pyrophosphate between the nitrogen and oxygen groups. The Gly³⁴ has an alpha amino group that also could interact with the same oxygen atom by a hydrogen bond. The Arg⁴¹ has two nitrogen atoms in the guanidino functional group that could form hydrogen bonds with an oxygen of the terminal phosphate group in farnesyl pyrophosphate and are expected to have nitrogen to oxygen interatomic distances of about 2.4 to 3.2 Å. The Arg⁷⁹ has a guanidino group having two oxygens that could interact with two oxygen atoms of the phosphate group of farnesyl pyrophosphate forming hydrogen bonds with inter-atomic distances of about 2.4 to 3.2 Å.

One aspect of the invention relates to compositions comprising Streptococcus undecaprenyl pyrophosphate synthase and a ligand in crystalline form. In general, the ligand can be a substrate, inhibitor, or co-factor. More specifically, the ligand can be selected from the group consisting of magnesium ion, farnesyl pyrophosphate, isopentyl pyrophosphate, sulfate ion, and any inhibitor that binds to a substrate binding site. The inhibitor can be any inhibitor of the synthase, including a low affinity or high affinity inhibitor. In one aspect the crystal comprises ligands, or parts thereof, having atomic coordinates according to FIG. 2, or portions thereof.

In another aspect the crystalline undecaprenyl pyrophosphate synthase comprises amino acid residues having atomic coordinates according to FIG. 2, or a substantial portion thereof. In such a crystal, the synthase is preferably a dimer of identical polypeptide chains. In one aspect, the invention comprises an amino acid sequence corresponding to SEQ ID NO:1. In another aspect, the undecaprenyl pyrophosphate synthase comprises an amino acid sequence corresponding to residues 21-252 of SEQ ID NO:1.

The invention also relates to first and second ligand binding sites of undecaprenyl pyrophosphate synthase. The first and second ligand binding sites are defined by amino acid residues that interact with the polar or ionic head group of the ligands and, optionally, with other amino acid residues that interact with a hydrophobic tail of the ligand. Alternatively, the amino acid residues of the binding sites can interact indirectly with the substrate, for example, by binding to a cofactor which in turn binds to a substrate, or by binding to another amino acid residue which in turn binds to a substrate.

The first ligand binding site can be defined as comprising at least one amino acid residue selected from the group consisting of Asp²⁸, Gly²⁹, Gly³¹, Arg³², Arg⁴¹, Ala⁷¹, Arg⁷⁹, Leu⁹⁰, Pro⁹¹, and Phe¹⁴³. In a preferred embodiment, the first ligand binding site comprises at least three of these amino acid residues. In a yet more preferred embodiment, the first ligand binding site comprises at least six of these amino acid residues. In a most preferred embodiment, the first ligand binding site comprises all ten amino acid residues.

The first ligand binding site can alternatively comprise at least about 80% of the amino acid residues selected from the group consisting of Asp²⁸, Gly²⁹, Gly³¹, Arg ³², Arg⁴¹, Ala⁷¹, Arg⁷⁹, Leu⁹⁰, Pro⁹¹, and Phe¹⁴³. In a preferred embodiment, the first ligand binding site comprises at least about 90% of the amino acid residues selected from the group consisting of Asp²⁸, Gly²⁹, Gly³¹, Arg³², Arg⁴¹, Ala⁷¹, Arg⁷⁹, Leu⁹⁰, Pro⁹¹, and Phe¹⁴³.

The second ligand binding site can be defined as comprising at least one amino acid residue selected from the group consisting of Asp²⁸, Arg²⁰⁰, Arg²⁰⁶, and Ser²⁰⁸ from one chain (A) of the dimer, and Glu²¹⁹ and Gly²⁵⁰ from the other chain (B) of the dimer. In a preferred embodiment, the second binding site comprises at least three of these amino acid residues. In a more preferred embodiment, the second binding site comprises all six of these amino acid residues.

The second ligand binding site can alternatively comprise at least about 80% of the amino acid residues selected from the group consisting of Asp²⁸, Arg²⁰⁰, Arg²⁰⁶, Ser²⁰⁸, Glu²¹⁹(B), and Gly²⁵¹(B). In a preferred embodiment, the second ligand binding site comprises at least about 80% of the amino acid residues selected from the group consisting of Asp²⁸, Arg²⁰⁰, Arg²⁰⁶, Ser²⁰⁸, Glu²¹⁹(B), and Gly²⁵¹(B).

Another aspect of the invention relates to methods of designing or identifying a potential ligand for an undecaprenyl pyrophosphate synthase, the method comprising using a three-dimensional structure including atomic coordinates of amino acid residues 28, 29, 31, 32, 41, 71, 91 and 143, according to FIG. 2. In a preferred embodiment, the coordinates are those of S. pneumoniae undecaprenyl pyrophosphate synthase, or a substantial portion thereof. The method can include obtaining the potential ligand which can include synthesizing the ligand in whole or in part, borrowing the ligand, and purchasing the ligand.

In one aspect the invention is directed to computational models of a composition comprising an undecaprenyl pyrophosphate synthase having atomic coordinates of S. pneumoniae undecaprenyl pyrophosphate synthase, or a portion thereof, and a computer program running on a computer addressing the atomic coordinates. The atomic coordinates can be those of FIG. 2, or a substantial portion thereof.

In another aspect, the invention is directed to methods of designing or identifying a ligand or a potential inhibitor of a second undecaprenyl pyrophosphate synthase comprising: (a) using a three-dimensional structure of a first undecaprenyl pyrophosphate synthase, as defined by atomic coordinates according to FIG. 2, or a substantial portion thereof; (b) identifying at least one first amino acid residue having a first peptide backbone and the amino acid residue(s) defining, in part, at least one ligand binding site; (c) employing protein alignment means to identify in the second undecaprenyl pyrophosphate synthase at least one second amino acid residue having a second peptide backbone that is capable of substantially aligning with the first backbone; (d) employing the three-dimensional structure to design or select the potential ligand for the second undecaprenyl pyrophosphate synthase; (e) synthesizing the potential ligand; and (f) contacting the potential ligand with the second undecaprenyl pyrophosphate synthase to determine binding to the second undecaprenyl pyrophosphate synthase; wherein the second amino acid residue differs from the first amino acid residue.

In yet another aspect, the invention is directed to computational models of an active site of an isolated undecaprenyl pyrophosphate synthase comprising a magnesium ion cofactor and a polypeptide comprising a first arginine residue having a guanidino group having nitrogen atoms, and an aspartic acid residue comprising oxygen atoms forming an acid functional group, wherein the oxygen atoms coordinate with the cofactor and at least one nitrogen atom of the guanidino group of the first arginine residue; and a second arginine residue in a polypeptide loop comprising the sequence Glu Asn Trp Xaa Arg Pro (SEQ ID NO:2). The Arg has at least one nitrogen atom capable of coordinating an atom of a ligand. Xaa is any amino acid residue, including hydrophilic, hydrophobic, and ionic amino acid residues. The cofactor, aspartic acid residue, first arginine residue, and second arginine residue form at least a part of an active site of an undecaprenyl pyrophosphate synthase. In one embodiment Xaa is Thr.

Moreover, the active site can further comprise a third arginine in an alpha-helix comprising the sequence Asp Gly Asn Xaa Arg (SEQ ID NO:3), the Arg having at least two nitrogen atoms capable of coordinating an atom of a ligand. Xaa in SEQ ID NO:3 is any amino acid residue, including hydrophilic, hydrophobic, and ionic amino acid residues. In one embodiment, Xaa is Gly.

The invention is also directed to computational models of an active site comprising a representation of the active site of undecaprenyl pyrophosphate synthase by a computer program capable of running on a computer.

In one aspect, the invention is directed to computational models of a composition comprising an undecaprenyl pyrophosphate synthase having at least twelve of the atomic coordinates of S. pneumoniae undecaprenyl pyrophosphate synthase and a computer program running on a computer addressing the atomic coordinates. Preferably, the model comprises at least twenty-four, more preferably at least 36 atomic coordinates, and most preferably at least 48 atomic coordinates.

The computational model can further comprise an amino acid residue sequence Asp Gly Asn Gly Arg Trp (SEQ ID NO:4), the Arg having at least one nitrogen atom, and a ligand comprising at least one oxygen atom, wherein the at least one nitrogen atom abuts the oxygen atom by about 2.4 Å.

In another embodiment, the computational model can further comprise an amino acid residue sequence Asp Gly Asn Gly Arg Trp (SEQ ID NO:4), each Gly having a nitrogen atom, and a ligand comprising at least one oxygen atom, wherein the nitrogen atom abuts the oxygen by about 3.3 Å.

In yet another embodiment, the computational model can alternatively further comprise an amino acid residue sequence Glu Asn Trp Thr Arg Pro (SEQ ID NO:5), the Arg having at least one nitrogen atom, and a ligand comprising at least one oxygen atom, wherein the nitrogen atom abuts the oxygen atom by about 2.9 Å.

In still another embodiment, the computational model can alternatively further comprise an amino acid residue sequence Pro Arg Val Phe Gly His (SEQ ID NO:6), the Arg having at least one nitrogen atom, and a ligand comprising at least one oxygen atom, wherein the nitrogen atom abuts the oxygen atom by about 3 Å.

Also, the computational model can alternatively further comprise an amino acid residue sequence Arg Leu Ser Asn Phe Leu (SEQ ID NO:7), the Ser having one nitrogen atom, and a ligand having at least one oxygen atom wherein the nitrogen atom abuts the oxygen atom by about 2.6 Å.

Design of Undecaprenyl Pyrophosphate Synthase Inhibitors

One of skill in the art can use any of a variety of known methods to screen chemical moieties for the ability to associate with undecaprenyl pyrophosphate synthase or with the Mg²⁺, FPP, or IPP binding sites that comprise part of the undecaprenyl pyrophosphate synthase active site. Visual inspection of a model of the ligand binding sites based on the undecaprenyl pyrophosphate synthase coordinates in FIG. 2 can lead to candidate chemical entities. Selected chemical moieties can then be positioned in orientations within one of the ligand binding sites of undecaprenyl pyrophosphate synthase. Positioning can be accomplished using software such as Quanta and Sybyl and is useful for changing the positions of chemical entities. Then standard molecular mechanics forcefields, such as CHARMM and AMBER can be used to minimize the energy and molecular kinetics of binding.

Other computer programs useful in selecting chemical moieties include:

1. DOCK (Kuntz et al., “A Geometric Approach to Macromolecule-Ligand Interactions”, J. Mol. Biol., 161:269-88 (1982)). DOCK is available from University of California, San Francisco, Calif.

2. GRID (Goodford, “A Computational Procedure for Determining Energetically Favorable Binding Sites on Biologically Important Macromolecules”, J. Med. Chem., 28:849-57 (1985)). GRID is available from Oxford University, Oxford, UK.

3. AUTODOCK (Goodsell and Olsen, “Automated Docking of Substrates to Proteins by Simulated Annealing”, Proteins: Structure. Function, and Genetics, 8:195-202 (1990)). AUTODOCK is available from Scripps Research Institute, La Jolla, Calif.

4. MCSS (Miranker and Karplus, “Functionality Maps of Binding Sites: A Multiple Copy Simultaneous Search Method.” Proteins: Structure. Function and Genetics, 11:29-34 (1991)). MCSS is available from Molecular Simulations, Burlington, Mass.

Selected moieties can be assembled into a single compound by initial visual review of the organization of the parts to make a whole in relation to the atomic coordinates of undecaprenyl pyrophosphate synthase. Model building with software such as Quanta or Sybyl can supplement the process.

Other programs useful in building chemical moieties into a ligand or inhibitor include:

1. CAVEAT (Bartlett et al, “CAVEAT: A Program to Facilitate the Structure-Derived Design of Biologically Active Molecules”. In “Molecular Recognition in Chemical and Biological Problems”, Special Pub., Royal Chem. Soc., 78:182-96 (1989)). CAVEAT is available from the University of California, Berkeley, Calif.

2. 3D Database systems such as MACCS-3D (MDL Information Systems, San Leandro, Calif.). See also, Martin, “3D Database Searching in Drug Design”, J. Med. Chem., 35:2145-54 (1992)).

3. HOOK (available from Molecular Simulations, Burlington, Mass.).

An undecaprenyl pyrophosphate synthase inhibitor or ligand can be prepared one moiety at a time, as described. Moreover, inhibitory or other undecaprenyl pyrophosphate synthase binding compounds can be designed “de novo” using either a vacant binding site or with moieties of a known inhibitor. Computer programs that support this approach include:

1. LEGEND (Nishibata and Itai, Tetrahedron, 47:8985 (1991)). LEGEND is available from Molecular Simulations, Burlington, Mass.

2. LUDI (Bohm, “The Computer Program LUDI: A New Method for the De Novo Design of Enzyme Inhibitors”, J. Comp. Aid. Molec. Design, 6:61-78 (1992)). LUDI is available from Biosym Technologies, San Diego, Calif.

3. LeapFrog (available from Tripos Associates, St. Louis, Mo.).

Variations on molecular modeling can be useful in this invention and include: Cohen et al., “Molecular Modeling Software and Methods for Medicinal Chemistry”, J. Med. Chem., 33:883-94 (1990); and Navia and Murcko, “The Use of Structural Information in Drug Design”, Current Opinions in Structural Biology, 2:202-10 (1992).

The efficiency of a model ligand binding to undecaprenyl pyrophosphate synthase can be evaluated and optimized by computation. For example, an effective undecaprenyl pyrophosphate synthase inhibitor can induce a relatively small deformation upon binding, that is, the energy in the bound and free states would be similar. Thus, in one embodiment undecaprenyl pyrophosphate synthase inhibitors should preferably have a deformation energy upon binding of about 8 kcal/mole or less. In the case where undecaprenyl pyrophosphate synthase inhibitors can bind to the synthase in more than one conformation the deformation binding energy is the difference between the average energy of the bound conformations less the energy in free solution. Further enhancement of binding can be achieved by computational repulsive charge interaction between the ligand and the synthase. In a similar manner, dipole-dipole interactions can be reduced. Advantageously, the net dipole-dipole and charge interactions between ligand and undecaprenyl pyrophosphate synthase favor binding.

Computer software useful to evaluate energies of deformation and of electrostatic repulsion and attraction include: Gaussian 92, revision C, M. J. Frisch, Gaussian, Inc., Pittsburgh, Pa. \mathring{c}1992; AMBER, version 4.0, P. A. Kollman, University of California at San Francisco, ĉ1994; QUANTA/CHARMM, Molecular Simulations, Inc., Burlington, Mass. ĉ1994; and Insight II/Discover (Biosysm Technologies Inc., San Diego, Calif. ĉ1994). These applications can be used on suitable workstations. Other hardware systems and software packages will be known to those skilled in the art.

A model undecaprenyl pyrophosphate synthase-binding compound can then be modified by changing functional groups to improve binding or inhibitory properties. The modified group can be similar to the size, volume and distribution of polar and hydrophobic functional groups as the model compound or it can differ. Modified compounds can be analyzed for fit to undecaprenyl pyrophosphate synthase by the computer modeling methods described above.

One aspect of the invention comprises a method of identifying an inhibitor capable of binding to and inhibiting the enzymatic activity of an undecaprenyl pyrophosphate synthase, comprising: (a) introducing into a suitable computer program information defining the binding site of the undecaprenyl pyrophosphate synthase comprising first atomic coordinates of amino acids capable of binding to a substrate, wherein the program displays the three-dimensional structure thereof; (b) creating a three dimensional model of a test compound in the computer program; (c) displaying and superimposing the model of the test compound on the structure of the active site; (d) assessing whether the test compound model fits spatially into the active site; (e) incorporating the test compound in a biological synthase activity assay; and (f) determining whether the test compound inhibits enzymatic activity in the assay.

The method can further comprise introducing into the computer program second atomic coordinates of water molecules bound to the substrate. Thereby, the free energy of binding of the potential inhibitor can include displacement of bound water.

In one embodiment, the method comprises introducing into the computer program an amino acid residue sequence of the synthase, or portion thereof. In one preferred embodiment, the method comprises introducing into the computer program third atomic coordinates of a first 3₁₀ helix of the synthase, comprising the sequence Asn-Trp-Thr (H3a, see Table 1B). In another embodiment, the method further comprises introducing into the computer program fourth atomic coordinates of at least one synthase structural element selected from the group consisting of an alpha helix, a second 3₁₀ helix, a strand of beta sheet, and a coil.

In yet another embodiment of the method, the undecaprenyl pyrophosphate synthase structural elements consist essentially of a coil and (a) a first beta sheet strand consisting of His Ile Gly Ile Ile Met (SEQ ID NO:8), or homolog thereof, and a second coil; (b) a first alpha helix consisting of Asn Gly Arg Trp Ala Lys (SEQ ID NO:9), or homolog thereof, and a third coil; (c) a second alpha helix consisting of Arg Val Phe Gly His Lys Ala Gly Met Glu Ala Leu Gln Thr Val Thr Lys Ala Ala Asn Lys Leu (SEQ ID NO:10), or homolog thereof, and a fourth coil; (d) a second beta sheet strand consisting of Val Ile Thr Val Tyr Ala (SEQ ID NO:11), or homolog thereof, and a fifth coil; (e) a first 3₁₀ helix consisting of Asn Trp Thr, and a sixth coil; (f) a third alpha helix consisting of Asp Gln Glu Val Lys Phe Ile Met Asn Leu Pro Val Glu Phe Tyr Asp Asn Tyr Val Phe Glu Leu His Ala (SEQ ID NO:12), or homolog thereof, and a seventh coil; (g) a third beta sheet strand consisting of Lys Ile Gln Met Ile (SEQ ID NO:13), or homolog thereof, and an eighth coil; (i) a fourth alpha helix consisting of Lys Gln Thr Phe Glu Ala Leu Thr Lys Ala Glu Glu Leu Thr (SEQ ID NO:14), or homolog thereof, and a ninth coil; (j) a fourth beta sheet strand consisting of Ile Leu Asn Phe Ala Leu (SEQ ID NO:15), or homolog thereof, and an tenth coil; (k) a fifth alpha helix consisting of Gly Arg Ala Glu Ile Thr Gln Ala Leu Lys Leu Ile Ser Gln Asp Val Leu (SEQ ID NO:16), or homolog thereof, and a eleventh coil; (1) a sixth alpha helix consisting of Glu Glu Leu Ile Gly (SEQ ID NO:17), or homolog thereof, and a twelfth coil; (m) a seventh alpha helix consisting of Phe Thr Gln His (SEQ ID NO: 18), or homolog thereof, and a thirteenth coil; (n) a fifth beta sheet strand consisting of Leu Ile Ile Arg (SEQ ID NO:19), or homolog thereof, and a fourteenth coil; (p) a sixth beta sheet strand consisting of Glu Leu Tyr Phe (SEQ ID NO:20), or homolog thereof, and a sixteenth coil; (q) a third 3₁₀ helix consisting of Trp Pro Asp, and a seventeenth coil; and/or (r) an eighth alpha helix consisting of Glu Ala Ala Leu Gln Glu Ala Ile Leu Ala Tyr Asn (SEQ ID NO:21), or homolog thereof, and an eighteenth coil.

In still another embodiment of the method, the second coil is connected to the first alpha helix, the third coil is connected to the second alpha helix, the fourth coil is connected to the second beta sheet strand, the fifth coil is connected to the first 3₁₀ helix, the sixth coil is connected to the third alpha helix, the seventh coil is connected to the third beta sheet strand, the eighth coil is connected to the fourth alpha helix, the ninth coil is connected to the fourth beta strand, the tenth coil is connected to the fifth alpha helix strand, the eleventh coil is connected to the sixth alpha helix, the twelfth coil is connected to the seventh alpha helix, the thirteenth coil is connected to the fifth beta strand, the fourteenth coil is connected to the second 3₁₀ helix, the fifteenth coil is connected to the sixth beta sheet strand, the sixteenth coil is connected to the third 3₁₀ helix, and/or the seventeenth coil is connected to the eighth alpha helix. In this description, the numerical adjectives, first, second and so forth, do not necessarily indicate a temporal or spatial order, but rather, serve merely to distinguish otherwise similarly named elements from one another.

As one skilled in the art can appreciate, knowledge of the three-dimensional structure allows solution, by the method of molecular replacement, of crystal structures of undecaprenyl pyrophosphate synthase bound to inhibitors, and use of the method of difference Fourier analysis to determine the bound conformation of the inhibitors. Knowledge of the bound conformation then allows for the design of inhibitors with better properties.

Likewise, knowledge of the three-dimensional structure allows the user to solve, by the method of molecular replacement, the structure of undecaprenyl pyrophosphate synthase from any other organism.

Again, knowledge of the three-dimensional structure allows the user to solve, by the method of molecular replacement, the structures of undecaprenyl pyrophosphate synthase mutants which may be used as probes of undecaprenyl pyrophosphate synthase activity.

### EXAMPLES

The following non-limiting examples are presented to further illustrate the invention.

### Example 1

### Design of an Inhibitor

The atomic coordinates of the polypeptide chains of S. pneumoniae undecaprenyl pyrophosphate synthase, as identified in FIG. 2, can be used in a computer to construct a three-dimensional model of the active site. A putative competitive inhibitor can be fit into a binding site on the enzyme. One such putative inhibitor is (2Z,6E,10E)-4-methyl-geranylgeranyl diphosphate (see Ohnuma et al., FEBS Lett., 257:71-74 (1989)). Modifications in the putative inhibitor can be made to prepare a virtual library of structurally related compounds. A docking program can then be used to evaluate interaction of each compound with the synthase, and to compare and rank the relative binding of the compounds to the synthase.

Compounds that appear to have relatively high affinity for the synthase can be obtained or synthesized and evaluated in a biochemical or biological assay. A suitable biological assay can be a measurement of growth by, for example, changes in turbidity of a bacterial suspension culture.

### Example 2

### Use of an Inhibitor of Undecaprenyl Pyrophosphate Synthase Activity to Identify Novel Ligands

Novel ligands capable of binding to an undecaprenyl pyrophosphate synthase substrate binding site can be identified by using a known inhibitor, for example, (S)-farnesyl thiopyrophosphate, or a substrate, for example, farnesyl pyrophosphate. Useful substrates of the synthase in addition to isoprenyl pyrophosphate (C₅PP) and farnesyl pyrophosphate (C₁₅PP) include C₂₀PP, C₂₅PP, C₃₀PP, C₃₅PP, C₄₀PP, C₄₅PP, and C₅₀PP, where the subscript denotes the number of carbon atoms in the isoprenoid chain. Properties of (S)-farnesyl thiopyrophosphate are described by Chen et al., J. Biol. Chem., 277:7369 (2002).

The atomic features of the known inhibitors or substrates are introduced into a suitable computer program that has information defining the substrate binding site. Typically, the information includes atomic coordinates of those amino acids that can bind to a known synthase substrate, such as are identified in FIG. 2. The computer program can then display the three-dimensional structure of the binding site. Then a three-dimensional model of a test compound can be created in the computer program.

A docking program can be used to dock the model of the test compound to the structure of the binding site. That is, the program fits the test molecule into the binding site, allowing for rotation of the bonds of the molecule to test the several conformation of the test molecule, and evaluates the quality of fit. Similarly, a three dimensional model of the substrate or of an inhibitor of the synthase can be created and docking information obtained. Then the docking parameters of the test compound can be compared to those of the substrate or of the known inhibitor. The docking program can then provide an output which can rank order the association parameters of each test or comparison molecule to the synthase.

In consequence, candidate compounds most likely to have high affinity for the binding site can be readily identified. Synthesis of the most potent test molecules, or otherwise obtaining them, can provide physical molecules for biochemical or biological analysis.

The method can optionally include introducing the atomic coordinates of those water molecules bound to the substrate, such that the coordinates are available to the computer program. Optionally, one skilled in the art can introduce into the computer program the atomic coordinates of at least one synthase structural element. Exemplary structural elements are an alpha helix, a 3₁₀ helix, a strand of beta sheet, and a coil. The 3₁₀ helix can have the sequence Asn Trp Thr, a part of a polypeptide loop that can engage the substrate.

The method can optionally also include incorporating the test compound in a biochemical synthase activity assay for a synthase; and then determining whether the test compound inhibits synthase activity in the assay. Suitable inhibitors can be further assessed in cell permeability studies, viability studies, and bacteremia studies, for example by biological assays.

### Example 3

### Undecaprenyl Pyrophosphate Synthase Assay

Undecaprenyl pyrophosphate synthase activity can be determined by standard methods. Measurement of synthase activity in vitro in the absence and presence of putative inhibitors can yield information on direct effects on the synthase. By comparison, measurements using viable bacteria in the absence and presence of putative inhibitors can yield information, when compared with in vitro analyses, of cell permeation. One skilled in the art will recognize that undecaprenyl pyrophosphate synthase substrates and close analogs thereof will be substantially cell impermeant under normal conditions.

One suitable method of in vitro analysis follows. [¹⁴C]-IPP (55 mCi/mmol) is incubated for up to 20 min at 25° C. in the presence of IPP (2-400 μM), FPP (0.2-10 μM), and synthase (0.01-0.1 μM) in a suitable buffered solution. A suitable buffered solution is 0.1% Triton X-100 in 50 mM KCl, 0.5 mM MgCl₂,100 mM KOH-HEPES, pH 7.5. To measure a rate, aliquots of the reaction mixture are removed at timed intervals and mixed with a solution of 10 mM EDTA to stop the reaction. The reaction products are extracted with 1-butanol, the phases separated, and the radioactive materials measured by scintillation counting. The butanol phase, which contains the undecaprenyl pyrophosphate, can be evaporated, and the pyrophosphate groups hydrolyzed in a solution of 20% propanol containing 4.4 units/ml acid phosphatase, 0.1 % Triton X-100, and 50 mM sodium acetate, pH 4.7. The resultant polyprenols are extracted with 1-hexane and spotted on a reversed-phase TLC plate and developed using acetone/water (19:1) as the mobile phase. The TLC plates are then analyzed by autoradiography.

One skilled in the art can measure synthase activity in vitro using the assay described above in the absence and presence of putative inhibitors.

Equivalents

While specific embodiments of the subject invention have been discussed, the above specification is illustrative and not restrictive. Many variations of the invention will become apparent to those skilled in the art upon review of this specification. The appended claims should be interpreted by reference to the claims, along with their full scope of equivalents, and the specification, along with such variations.

All publications and patents mentioned herein, including those items listed below, are hereby incorporated by reference in their entirety as if each individual publication or patent was specifically and individually indicated to be incorporated by reference. In case of conflict, the present application, including any definitions herein, will control.

We claim:

1. Streptococcus undecaprenyl pyrophosphate synthase in crystalline form.

2. A composition comprising the synthase of claim 1.

3. The synthase of claim 1 wherein the synthase comprises an amino acid sequence at least about 80% homologous to SEQ ID NO:1.

4. The synthase of claim 3 wherein the synthase is at least about 90% homologous to SEQ ID NO:1.

5. The synthase of claim 1 wherein the synthase comprises a first ligand binding site, a second ligand binding site, or both.

6. The synthase of claim 3 wherein the synthase comprises a first ligand binding site, a second ligand binding site, or both.

7. The synthase of claim 4 wherein the synthase comprises a first ligand binding site, a second ligand binding site, or both.

8. The synthase of any one of claims 5, 6, or 7, comprising at least one ligand.

9. The synthase of claim 8 wherein synthase is co-crystallized with said ligand.

10. The synthase of claim 8 wherein the ligand is selected from the group consisting of farnesyl pyrophosphate, (S)-farnesyl thiopyrophosphate, isoprenyl pyrophosphate, magnesium ion, and sulfate ion.

11. The synthase of any one of claims 5, 6, or 7, wherein the first ligand binding site is defined by at least one amino acid residue selected from the group consisting of Asp²⁸, Gly²⁹, Gly³¹, Arg³², Arg⁴¹, Ala⁷¹, Arg⁷⁹, Leu⁹⁰, Pro⁹¹, and Phe¹⁴³.

12. The synthase of claim 5 wherein the first ligand binding site comprises at least about 80% of the amino acid residues selected from the group consisting of Asp²⁸, Gly²⁹, Gly³¹, Arg³², Arg⁴¹, Ala⁷¹, Arg⁷⁹, Leu⁹⁰, Pro⁹¹, and Phe¹⁴³.

13. The synthase of claim 5 wherein the first ligand binding site comprises at least about 90% of the amino acid residues selected from the group consisting of Asp²⁸, Gly²⁹, Gly³¹, Arg³², Arg⁴¹, Ala⁷¹, Arg⁷⁹, Leu⁹⁰, Pro⁹¹, and Phe¹⁴³.

14. The synthase of any one of claims 5, 6, or 7, wherein the second ligand binding site is defined by at least one amino acid residue selected from the group consisting of Asp²⁸, Arg²⁰⁰, Arg²⁰⁶, Ser²⁰⁸, Glu²¹⁹(B), and Gly²⁵¹(B).

15. The synthase of claim 5 wherein the second ligand binding site comprises at least about 80% of the amino acid residues selected from the group consisting of Asp²⁸, Arg²⁰⁰, Arg²⁰⁶, Ser²⁰⁸, Glu²¹⁹(B), and Gly²⁵¹(B).

16. The synthase of claim 5 wherein the second ligand binding site comprises at least about 90% of the amino acid residues selected from the group consisting of Asp²⁸, Arg²⁰⁰, Arg²⁰⁶, Ser²⁰⁸, Glu²¹⁹(B), and Gly²⁵¹(B).

17. The synthase of any one of claims 5, 6, or 7, wherein the first binding site comprises Asp²⁸, Gly²⁹, Gly³¹, Arg³², Arg⁴¹, Ala⁷¹, Arg⁷⁹, Leu⁹⁰, Pro⁹¹, and Phe¹⁴³, the second binding site comprises Arg²⁰⁰, Arg²⁰⁶, Ser²⁰⁸, Glu²¹⁹(B), and Gly²⁵¹(B), or both.

18. The synthase of any one of claims 5, 6, 7, 12, or 13, wherein the first ligand binding site is capable of associating with farnesyl pyrophosphate, (S)-farnesyl thiopyrophosphate, or both.

19. The synthase of any one of claims 5, 6, 7, 15, or 16, wherein the second ligand site is capable of associating with isoprenyl pyrophosphate, sulphate, or both.

20. The synthase of claim 1 wherein the synthase comprises a first ligand binding site defined by amino acid residues 28, 29, 31, 32, 41, 71, 79, 90, 91, 143 having atoms having atomic coordinates according to FIG. 2.

21. The synthase of claim 1 wherein the synthase comprises a second ligand binding site defined by amino acid residues 28, 200, 206, 208, 219(B), and 250(B) having atoms having atomic coordinates according to FIG. 2.

22. The synthase of claim 1 wherein the synthase comprises S. pneumoniae undecaprenyl pyrophosphate synthase.

23. A composition comprising Streptococcus undecaprenyl pyrophosphate synthase and a substrate or substrate analog in crystalline form.

24. The composition of claim 23 wherein the substrate or substrate analog is co-crystallized with the synthase.

25. The composition of claim 23 wherein the substrate is farnesyl pyrophosphate.

26. The composition of claim 23 wherein the substrate is isoprenyl pyrophosphate.

27. The composition of claim 23 wherein the substrate analog is (S)-farnesyl thiopyrophosphate.

28. A method for identifying a potential ligand for an undecaprenyl pyrophosphate synthase, comprising:
(a) using a three-dimensional structure of the synthase as defined by at least atomic coordinates of amino acid residues 28, 29, 31, 32, 41, 71, 79, 90, 91, and 143 according to FIG. 2;
(b) employing the three-dimensional structure to design or select the potential ligand;
(c) obtaining the potential ligand; and
(d) contacting the potential ligand with the undecaprenyl pyrophosphate synthase to determine binding of the potential ligand to the synthase.

29. The method of claim 28 further comprising:
(e) identifying chemical entities or fragments thereof capable of binding to the synthase; and
(f) assembling the identified chemical entities or fragments thereof into a single molecule to provide the structure of the potential ligand.

30. The method of claim 28 wherein the ligand is an inhibitor.

31. The method of claim 30 wherein the inhibitor is a competitive inhibitor.

32. The method of claim 30 wherein the inhibitor is a noncompetitive inhibitor.

33. The method of claim 28 wherein the ligand is designed de novo.

34. The method of claim 28 wherein the ligand is designed from a known inhibitor.

35. The method of claim 28 wherein said synthase includes a ligand bound thereto, and the atomic coordinates, or a portion thereof, of said ligand are used according to step (a).

36. The method of claim 28 wherein the three-dimensional structure is further defined by atomic coordinates of amino acid residues 200, 206, and 208 according to FIG. 2.

37. The method of claim 28 wherein the three-dimensional structure is further defined by atomic coordinates of amino acid residues 219(B) and 250(B) according to FIG. 2.

38. The method of claim 28 wherein the potential ligand is designed to form a hydrogen bond with at least one amino acid residue selected from the group consisting of Gly²⁹, Gly³¹, Arg³², Arg⁴¹, and Arg⁷⁹.

39. The method of claim 28 wherein the potential ligand is designed to form a hydrogen bond with at least one amino acid residue selected from the group consisting of Arg²⁰⁰, Arg²⁰⁶, Ser²⁰⁸, Glu²¹⁹(B), and Gly²⁵¹(B).

40. The method of claim 28 wherein the potential ligand is designed to form a hydrophobic bond with at least one amino acid residue selected from the group consisting of Ala⁷¹, Leu⁹⁰, Pro⁹¹, and Phe¹⁴³.

41. The method of claim 28 wherein (c) precedes (b).

42. A method for identifying a potential inhibitor of a mutant undecaprenyl pyrophosphate synthase, comprising:
(a) using a three-dimensional structure of undecaprenyl pyrophosphate synthase as defined by atomic coordinates of undecaprenyl pyrophosphate synthase according to FIG. 2;
(b) replacing one or more undecaprenyl pyrophosphate synthase amino acids selected from 28, 29, 31, 32, 41, 71, 79, 90, 91, 143, 200, 206, 208, 219, and 250 of SEQ ID NO:1 in the three-dimensional structure with a different naturally occurring amino acid, thereby forming a mutant undecaprenyl pyrophosphate synthase;
(c) employing the three-dimensional structure to design or select the potential inhibitor; and
(d) contacting the potential inhibitor with the mutant undecaprenyl pyrophosphate synthase or the undecaprenyl pyrophosphate synthase in the presence of a substrate to test the ability of the potential inhibitor to inhibit the undecaprenyl pyrophosphate synthase or the mutant undecaprenyl pyrophosphate synthase.

43. The method of claim 42 wherein the potential inhibitor is selected from a database.

44. The method of claim 42 wherein the potential inhibitor is designed de novo.

45. The method of claim 42 wherein the potential inhibitor is designed from a known inhibitor.

46. The method of claim 42 wherein step (c) comprises:
(i) identifying chemical entities or fragments thereof capable of associating with the mutant undecaprenyl pyrophosphate synthase; and
(ii) assembling the identified chemical entities or fragments thereof into a single molecule to provide the structure of the potential inhibitor.

47. The method according to any one of claims 42-46 wherein the potential inhibitor is a competitive inhibitor of mutant undecaprenyl pyrophosphate synthase.

48. The method according to any one of claims 42-46 wherein the potential inhibitor is a non-competitive or uncompetitive inhibitor of mutant undecaprenyl pyrophosphate synthase.

49. A method of identifying a ligand capable of binding to an undecaprenyl pyrophosphate synthase substrate binding site, comprising:
(a) introducing into a suitable computer program information defining the binding site comprising first atomic coordinates of amino acids capable of binding to a synthase substrate, wherein the program displays the three-dimensional structure of the binding site;
(b) creating a three dimensional model of a test compound in the computer program;
(c) docking the model of the test compound to the structure of the binding site;
(d) creating a second three dimensional model of the substrate or an inhibitor of the synthase and docking the second model thereto; and
(e) comparing the docking of the test compound and of the substrate or the inhibitor of the synthase to provide an output of the program.

50. The method of claim 49 further comprising introducing into the computer program second atomic coordinates of water molecules bound to the substrate.

51. The method of claim 49 further comprising introducing into the computer program third atomic coordinates of at least one synthase structural element selected from the group consisting of an alpha helix, a 3₁₀ helix, a strand of beta sheet, and a coil.

52. The method of claim 51 wherein the 3₁₀ helix comprises the sequence Asn-Trp-Thr.

53. The method of claim 49 further comprising:
(f) incorporating the test compound into a biological or biochemical synthase activity assay; and
(g) determining whether the test compound inhibits synthase activity in the assay.

54. A method for identifying a potential inhibitor for an undecaprenyl pyrophosphate synthase, comprising:
(a) using a three-dimensional structure of the synthase as defined by atomic coordinates of undecaprenyl pyrophosphate synthase according to FIG. 2;
(b) employing said three-dimensional structure to design or select the potential inhibitor; and
(c) contacting the potential inhibitor with the synthase in the presence of a substrate to determine the ability of the potential inhibitor to inhibit the synthase.

55. The method of claim 54 wherein the potential inhibitor is designed de novo.

56. The method of claim 54 wherein the potential inhibitor is designed from a known inhibitor.

57. The method of claim 54 wherein step (b) comprises:
(i) identifying chemical entities or fragments thereof capable of associating with the synthase; and
(ii) assembling the identified chemical entities of fragments into a single molecule to provide the structure of the potential inhibitor.

58. The method of claim 57 wherein the potential inhibitor is designed de novo.

59. The method of claim 57 wherein the potential inhibitor is designed from a known inhibitor.

60. The method according to any one of claims 54 or 57-59, wherein the potential inhibitor is a competitive inhibitor of undecaprenyl pyrophosphate synthase.

61. The method according to any one of claims 54 or 57-59, wherein said potential inhibitor is a non-competitive or uncompetitive inhibitor of undecaprenyl pyrophosphate synthase.

62. A method for drug design comprising using atomic coordinates of a S. pneumoniae undecaprenyl pyrophosphate synthase having at least one ligand binding site to computationally evaluate relative associations of chemical entities with the ligand binding site and produce an output.

63. The method of claim 62 wherein the chemical entity is an intermediate in a farnesyl pyrophosphate elongation reaction, or an analog thereof.

64. A method for solving a crystal form comprising using atomic coordinates of a S. pneumoniae undecaprenyl pyrophosphate synthase crystal or portions thereof, to solve a crystal form of a mutant, homolog or co-complex of the undecaprenyl pyrophosphate synthase by molecular replacement.

65. The method of claim 64 comprising using atomic coordinates of a ligand bound to the undecaprenyl pyrophosphate synthase.

66. A machine-readable data storage medium comprising a data storage material encoded with machine-readable data comprising atomic coordinates comprising amino acid residues 28, 29, 31, 32, 41, 71, 79, 90, 91, and 143 according to FIG. 2.

67. The machine-readable data storage medium of claim 66 wherein the machine-readable data comprise atomic coordinates comprising at least one amino acid residue selected from the group consisting of 200, 206, 208, 219(B), and 250(B) according to FIG. 2.

68. The machine-readable data storage medium of claim 66 wherein the machine-readable data comprise the three-dimensional structure of S. pneumoniae undecaprenyl pyrophosphate synthase.

69. A computer-implemented tool for design of a drug, comprising:
(a) a three-dimensional structure of an undecaprenyl pyrophosphate synthase as defined by atomic coordinates of a S. pneumoniae undecaprenyl pyrophosphate synthase having at least one ligand binding site;
(b) a model of a chemical entity; and
(c) a computer program addressing the coordinates and capable of modeling the chemical entity in the ligand binding site to produce an output.

70. The tool of claim 69, wherein said atomic coordinates are essentially as described in FIG. 2.

71. A computer for producing a three-dimensional representation of an undecaprenyl pyrophosphate synthase ligand binding site comprising:
(a) a machine-readable data storage medium comprising a data storage material encoded with machine-readable data comprising the atomic coordinates comprising the amino acid residues 28, 29, 31, 32, 41, 71, 79, 90, 91, and 143 according to FIG. 2;
(b) a working memory for storing instructions for processing the machine-readable data;
(c) a central-processing unit coupled to the working memory and to the machine-readable data storage medium for processing the machine readable data into the three-dimensional representation; and
(d) a display coupled to the central-processing unit for displaying the three-dimensional representation.

72. The computer of claim 71 wherein the computer produces a three-dimensional representation of the ligand binding site of an undecaprenyl pyrophosphate synthase; and wherein the machine-readable data comprises the atomic coordinates of the ligand binding site.

73. A method of preparing Streptococcus undecaprenyl pyrophosphate synthase in crystalline form comprising incubating the synthase in a hanging drop.
